(19) United States
(12) Patent Application Publication
(10) Pub. No.: US 2015/0004237 A1
(43) Pub. Date: Jan. 1, 2015

(54) CELLULOSE DERIVATIVES FOR INHIBITING CRYSTALLIZATION OF POORLY WATER-SOLUBLE DRUGS

(71) Applicants: Virginia Tech Intellectual Properties, Inc., Blacksburg, VA (US); Purdue Research Foundation, West Lafayette, IN (US)

(72) Inventors: Kevin J. Edgar, Blacksburg, VA (US); Bin Li, Blacksburg, VA (US); Lynne Taylor, West Lafayette, IN (US); Grace Ilevbare, West Lafayette, IN (US); Stephanie M. Williams, Cleveland Heights, OH (US); Haoyu Liu, Blacksburg, VA (US)

(21) Appl. No.: 14/368,911

(22) PCT Filed: Jan. 9, 2013
(86) PCT No.: PCT/US13/20835

§ 371 Date: Jun. 26, 2014

Related U.S. Application Data
(60) Provisional application No. 61/718,111, filed on Oct. 24, 2012.
(60) Provisional application No. 61/624,030, filed on Apr. 13, 2012.
(60) Provisional application No. 61/584,547, filed on Jan. 9, 2012.

(51) Int. Cl. A61K 47/38; A61K 31/427; A61K 31/357; A61K 31/536; A61K 31/40; A61K 31/05; C08B 3/00; A61K 31/352

(52) U.S. Cl. CPC A61K 47/38; C08B 3/00; A61K 31/427; A61K 31/352; A61K 31/536; A61K 31/40; A61K 31/05; A61K 31/357
USPC 424/489; 514/781; 536/63; 514/365; 514/456; 514/230.5; 514/406; 514/733; 514/453

(57) ABSTRACT

Provided are cellulose esters useful for inhibiting solution crystallization of drugs. Specific polymers include cellulose esters of formula I:

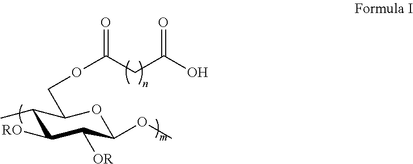

wherein n of the ω-carboxyalkanoyl group,

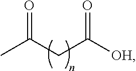

is 3, 4, 6, or 8 to provide a ω-carboxyalkanoyl group chosen from succinoyl, glutaroyl, adipoyl, sebacyl, and suberyl groups; and wherein R is chosen from: a hydrogen atom; and an alkanoyl group chosen from acetyl, propionyl, butyryl, valeroyl, hexanoyl, nonanoyl, decanoyl, lauroyl, palmitoyl, and stearoyl groups; wherein there is a total degree of substitution of the alkanoyl group and the ω-carboxyalkanoyl group of at least 2.0; and wherein the polymer comprises m repeating units where n=1 to 1,000,000, or 10 to 100,000, or 100 to 1,000, such as 1 to 6,000. Embodiments further include compositions comprising cellulose esters and poorly water-soluble drugs, which compositions exhibit greater solubility and stability in solution as compared to the drugs alone.

### CROSS-REFERENCE TO RELATED APPLICATIONS

This application relies on the disclosure of and claims the benefit of the filing date of U.S. Provisional Application Nos. 61/584,547, filed Jan. 9, 2012; 61/624,030 filed Apr. 13, 2012; and 61/718,111 filed Oct. 24, 2012, the disclosures of which are each incorporated by reference herein in their entireties.

### BACKGROUND OF THE INVENTION

1. Field of the Invention

The present invention relates to the fields of chemistry and pharmaceuticals. Embodiments of the invention provide cellulose esters useful for inhibiting solution and solid phase crystallization of drugs. Embodiments further include compositions comprising cellulose esters and poorly water-soluble drugs, which compositions exhibit greater solution concentration and stability in solid phase and in solution as compared to the drugs alone. In many cases this enhanced stability and solution concentration leads to enhanced oral bioavailability. Methods for making and using the compounds and compositions are also included within the scope of the invention.

2. Description of Related Art

Due to the poor aqueous solubility of many new drug candidates, supersaturating dosage forms such as amorphous drug-polymer blends (often termed solid dispersions), are finding increasing application. For this approach to be effective, it is important to inhibit solution crystallization from the supersaturated solution generated upon dissolution of the amorphous formulation, since crystallization of the drug after release but prior to absorption into the enterocytes may negate the advantages of the supersaturating formulation.

Solubility is a physicochemical parameter of a new molecule that should be assessed and understood very early on in drug discovery and drug candidate selection. See, e.g., S. Stegeman, F. Leveiller, D. Franchi, H. de Jong, H, Lindén, “When solubility becomes an issue: from early stage to proof of concept,” European J. Pharm. Sciences: 31, 249-261 (2007). In the discovery phase of drug development, high throughput methods have generated new drug candidates that tend to be hydrophobic and poorly water soluble. The problem of poor aqueous solubility is critical since solubility is a prerequisite for therapeutic activity. If a drug candidate has reasonable membrane permeability, then often the rate-limiting process in absorption is the drug dissolution step. See C. W. Pouton, “Formulation of poorly water-soluble drugs for oral administration: Physicochemical and physiological issues and the lipid formulation classification system,” European J. Pharm. Sciences, 29: 278-287 (2006). Class II compounds in the biopharmaceutical classification system (BCS) have this characteristic.

Supersaturation as a means to provide higher thermodynamic activity may enhance the absorption of a drug over and above that of a simple solution. See Warren, D., Benameur, H., Porter, C. J. H., Pouton, C. W., “Using polymeric precipitation inhibitors to improve the absorption of poorly water-soluble drugs: A mechanistic basis for utility,” Journal of Drug Targeting, 18(10): 704-731 (2010). Additionally, the speed at which drug precipitation from supersaturated solutions occurs dictates whether the use of the metastable amorphous form will be beneficial in increasing bioavailability. Crystallization of a drug from solution involves two processes, nucleation and growth. If one or both processes can be retarded or inhibited, supersaturation will be maintained for a physiologically sufficient time period, in turn absorption is enhanced. A number of polymeric additives have been identified in the literature as effective crystallization inhibitors and the effectiveness of polymers depends on their ability to interact with the drug through mechanisms such as adsorption onto small crystallites or amorphous solids. The nature and extent of adsorption and other drug-polymer interactions may be directly related to the extent of inhibition.

Low aqueous solubility compounds often suffer from limited bioavailability and the formulation of these molecules into orally administered dosage forms with sufficient bioavailability is a drug delivery challenge. Several solubility enhancing formulation strategies are routinely used to create elevated concentrations of the drug in the GI tract including: cosolvents, complex forming agents such as cyclodextrins, and surfactant-based formulations. Although significant increased apparent solubility may be achieved by these techniques, their impact on the fraction of the overall dose that is absorbed is erratic. Additionally, it has been demonstrated that cosolvent, complexation and surfactant-based solubilization methods may lead to lower effective permeability; cyclodextrins and surfactants can decrease the free fraction of drug which results in decreased intestinal membrane permeability of lipophilic drugs (BCS class II) (see Miller, J. M.; Beig, A.; Carr, R. A.; Spence, J. K.; Dahan, A. “A Win-Win Solution in Oral Delivery of Lipophilic Drugs: Supersaturation via Amorphous Solid Dispersions Increases Apparent Solubility without Sacrifice of Intestinal Membrane Permeability,” Molecular Pharmaceutics 9(7), 2009-2016 (2012)), while the permeability of drugs solubilized in cosolvents has been found to decrease with increasing cosolvent fraction.

Modifications to stable crystal structures, for example amorphous forms of the drug, can increase aqueous drug solubility without reducing intestinal membrane permeability. The supersaturated solutions generated from the use of amorphous solids may lead to an increase in absorption compared to that of a saturated solution if supersaturation can be maintained for a physiologically-relevant type period. Using in silico modeling and simulation to predict drug absorption from the GI tract, the relationship between drug supersaturation and improved oral bioavailability has been demonstrated. However, in order to maintain supersaturation, crystallization—nucleation and/or crystal growth—should be prevented. Trace crystalline material in an amorphous formulation, either resulting from the manufacturing process or produced during storage, can thus potentially have a significant impact on the extent and duration of supersaturation; the presence of an effective crystal growth inhibitor in solution is therefore highly desirable to prolong supersaturation. It has been proposed that maintenance of supersaturation through the addition of polymeric additives is the result of intermolecular interactions in solution (hydrogen bonding) and/or steric hindrance of recrystallization.

Preventing nucleation may not always be possible and inhibition of growth may be necessary to maintain supersaturation and thus bioavailability. For example, the amorphous formulation may contain seed crystals resulting from the manufacturing process, which will lead to rapid de-supersaturation unless effective crystal growth inhibitors have been included in the formulation. Small traces of crystalline material can thus potentially have a significant impact on the extent and duration of supersaturation. It is therefore important to understand the underlying factors that affect the ability of polymeric additives to inhibit crystal growth for a given drug compound so as to enable the rational selection of formulation components.

The use of polymers as precipitation inhibitors has also rekindled interest in amorphous solid dispersions. Polymers are a vital part of solid dispersions and are used to stabilize high energy forms, such as amorphous materials. However, the introduction of water into amorphous systems upon storage or during dissolution results in an increase in mobility and disruption of specific interactions between drug and polymers, amongst other factors. These factors increase the likelihood for crystallization. In addition, the rate of release and the release mechanism of drug from amorphous solid dispersions will dictate whether increased solution concentration will be attained within a physiologically relevant time period.

Even though stabilization of supersaturated solutions by polymeric additives has been the focus of numerous studies, the underlying mechanism(s) by which this process occurs has not been fully described. Therefore it is imperative to understand the principal factors that are responsible for the improved physical stability in the presence of polymers, so that the most appropriate polymers can be selected to improve the bioavailability of a given drug. Knowledge of the key mechanism(s) will help scientists design effective polymeric additives that will inhibit crystallization from solution.

### SUMMARY OF THE INVENTION

To enhance drug solubility and thus bioavailability of various compounds, the inventors have created a superior family of polymers which can be used in formulating drugs into amorphous solid dispersions with these polymers. This family of polymers provides excellent stabilization of the high-energy amorphous drug in the solid phase amorphous dispersion, and stabilization of the drug against crystallization (and in some cases against chemical degradation) after release into aqueous solution in the gastrointestinal lumen but before the drug permeates through the gastrointestinal epithelium. This family of polymers can also be formulated to adjust the release rate to meet therapeutic needs, in some cases by blending with other polymers that possess complimentary properties, for example enhanced aqueous solubility.

An object of the invention thus provides polymers and polymer combinations for stabilizing a drug or drug combination in solution and in the solid phase. Preferred polymers are useful for stabilizing a poorly soluble drug or drug combination in solution and in the solid phase. Even further, embodiments of the invention provide polymer and polymer combinations that are useful for stabilizing an amorphous drug or drug combination in solution and in the solid phase. Embodiments of the invention also include using one or more polymers with any drug or drug combination to increase solubility or stability of the drug(s) in solution, regardless of the solubility of the drug alone. That is, polymers of the invention are not limited to use with poorly soluble drugs.

In particular, provided are cellulose esters of formula I:

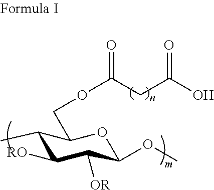

wherein n=2, 3, 4, 6, or 8 (alkyl groups in the ω-carboxyalkanoyl group) to provide a ω-carboxyalkanoyl group chosen from succinoyl, glutaroyl, adipoyl, sebacyl, and suberyl groups;

wherein R is chosen from: a hydrogen atom; and an alkanoyl group chosen from acetyl, propionyl, butyryl, valeroyl, hexanoyl, nonanoyl, decanoyl, lauroyl, palmitoyl, and stearoyl groups; and

wherein there is a total degree of substitution of the alkanoyl group and the ω-carboxyalkanoyl group of at least 2.0; and

wherein m number of repeating units in the polymer ranges from 1-1,000,000, such as from 10 to 100,000, or from 100 to 1,000, such as 1-6,000.

Embodiments of the invention include methods of making such polymers, which include synthesis methods according to Scheme I provided in this specification.

Objects of embodiments of the invention also provide compositions comprising one or more cellulose esters containing ω-carboxyalkanoyl groups, one or more drugs (preferably a drug in need of solubility enhancement), optionally a second more water-soluble polymer, and such additives that may be necessary for other purposes (such as mold release aids and colorants).

Such compositions can be formulated by one of several methods; coextrusion, or dissolution in a common solvent followed by one of several processes; coprecipitation into a non-solvent for all components, spray-drying, lyophilization, or evaporation. Preferably, the compositions are characterized by one or more of the following features: 1) stabilization of the drug against crystallization for extended time periods (>1 yr) in the solid phase; 2) stabilization of the drug against crystallization for up to 24 h after the drug dissolves in water or in gastrointestinal fluid; 3) in certain cases, where the drug is chemically unstable in aqueous solution, stabilization for up to 24 h against chemical degradation; 4) drug release from the formulation at a therapeutically effective rate.

Polymers of the invention, including CAP Adp 3X, CAB Adp 3X and CAP 504-0.2 Adp are effective in inhibiting nucleation of ritonavir. These polymers were more effective than the commercially available polymers, HPMC and HPMCAS. Likewise it has been found that CAP Adp 3X and CAB Adp 3X were among the most effective in inhibiting crystal growth of ritonavir, while CA Sub and CA Seb are even better.

Hydrophobicity is an important factor in crystal growth inhibition. Hydrophobicity of the polymer may affect the extent of adsorption of polymer to the crystal surface and in turn may influence the effectiveness of the polymer as a crystal growth inhibitor. Polymers with an effective level of hydrophilic/hydrophobic balance include CAP Adp 3X and CAB Adp 3X, and are more effective in inhibiting crystal growth compared to more hydrophilic (CP Adp) and hydrophobic (CAP Seb) polymers.

The ability of the polymer to inhibit crystal growth decreases with decreasing degree of substitution of the adipate substituent. The higher the DS, the higher the number of anionic groups in solution, which in turn enhances effective adsorption of polymer to drug.

Decreasing the pH of the test medium from pH 6.8 to 3.8 reduces the degree of ionization of the novel polymer which results in a change of polymer conformation. This change in polymer conformation is believed to influence the inhibitory capacity of the novel polymers. The polymer coils up on itself, thus reducing the effectiveness of the adsorbed polymer on the drug crystal surface.

Although CAP 504-0.2 Adp is an excellent crystallization inhibitor, when formulated as a solid dispersion, it releases hydrophobic drugs slowly relative to the transit time for dosage forms through the upper GI tract. This problem was overcome by combining CAP 504-0.2 Adp with a hydrophilic polymer, PVP. The binary combination of these polymers resulted in an increase in dissolution rate and prolonged duration of supersaturation.

Further objects of the present invention include compositions of poorly soluble drugs (solubility<1 mg/mL), cellulose esters of structure 1 in which R is selected from among hydrogen, alkanoyl (acetyl, propionyl, butyryl, valeroyl, hexanoyl, nonanoyl, decanoyl, lauroyl, palmitoyl, and stearoyl), and ω-carboxyalkanoyl (succinoyl, glutaroyl, adipoyl, sebacyl, suberyl), in which the ω-carboxyalkanoyl composes at least a degree of substitution (DS) of 0.05 and can be 1 or near 1, and the total degree of substitution of alkanoyl and ω-carboxyalkanoyl is at least 2.0, and in which the drug is amorphous as determined by techniques including differential scanning calorimetry (DSC), X-ray diffraction (XRD), or X-ray powder diffraction (XRPD). Indeed, the drugs, polymers, and/or compositions of the invention can be evaluated and characterized by any one or more analytical techniques, including but not limited to XRD, XRPD, FT-IR, DSC and/or NMR spectroscopy.

Compositions of embodiments of the invention also include such compositions in which the solubility of the drug in buffer solutions that simulate contents of the small intestine (pH 6.8 phosphate buffer) is enhanced. Such compositions can also include those in which a second polymer is included which enhances the rate of release of the drug.

Compositions further include those comprising a second polymer. Preferably the second polymer is more water soluble than the first polymer (i.e., cellulose ester containing ω-carboxyalkanoyl groups). The second polymer, for example, can be chosen from the following poly(vinylpyrrolidinone) (PVP), hydroxypropyl methylcellulose (HPMC), poly(ethylene glycol) (PEG), and poly(propylene glycol) (PPG).

Compositions of the invention may be characterized in that the drug remains amorphous as determined by XRD and/or DSC for at least 1 year, 2 years, 3 years, 4 years, or even up to 10 years.

In compositions of embodiments of the invention, upon dispersion in buffer solutions that simulate the small intestine (pH 6.8 phosphate buffer), the drug dissolves to a maximum concentration, and at least 90% of that concentration is maintained for at least 24 h.

Preferred compositions include those in which the drug is chosen from among the following: ritonavir, efavirenz, etravirine, celecoxib, and clarithromycin. Such compositions may be useful for treating infectious diseases, for example tuberculosis or AIDS.

Compositions of the invention can also include those in which the drug is chosen from among the following: curcumin, ellagic acid, quercetin, naringenin, and resveratrol. Such compositions may be useful for treating or preventing cancer, among other diseases.

Although the compositions can be used for any drug, even drugs that possess sufficient solubility themselves, preferred compositions are those comprising a drug which is characterized by low solubility. Especially preferred compositions include those which comprise a drug from any Class II or Class IV type compounds. Even further preferred compositions include but are not limited to those comprising a drug from one or more of the following classes: antihypertensives, antianxiety agents, anticlotting agents, anticonvulsants, blood glucose lowering agents, decongestants, antihistamines, antitussives, antineoplastics, beta blockers, anti-inflammatories, antipsychotic agents, cognitive enhancers, cholesterol reducing agents, triglyceride reducing agents, anti-atherosclerotic agents, anti-obesity agents, autoimmune disorder agents, anti-impotence agents, antibacterial and anti-fungal agents, hypnotic agents, anti-Parkinsonism agents, anti-Alzheimer's disease agents, antibiotics, antidepressants, antiviral agents, glycogen phosphorylase inhibitors, protease inhibitors, anti-cancer drugs, and cholesteryl ester transfer protein inhibitors, and compounds which are useful for treating the underlying disease, or symptoms of such diseases that these classes of compounds are useful for treating.

### BRIEF DESCRIPTION OF THE DRAWINGS

The accompanying drawings illustrate certain aspects of some of the embodiments of the present invention, and should not be used to limit or define the invention. Together with the written description, the drawings serve to explain certain principles of embodiments of the invention.

FIGS. 1A-B are schematic diagrams illustrating the molecular structure of representative cellulose derivatives that can be used in compositions of the present invention.

FIG. 2A is a graph showing dissolution of amorphous ritonavir film.

FIG. 2B is a graph showing dissolution of the powder form of amorphous ritonavir prepared by the melt-quench method.

FIG. 3 is a graph of the nucleation-induction time of ritonavir at an initial concentration of 20 μg/mL, in the absence and presence of polymers at 5 μg/mL.

FIG. 4 is a graph showing the crystal growth rate of ritonavir in the presence of polymers with the y-axis being a ratio of the growth rate of ritonavir in the absence of polymer to growth rate of ritonavir in the presence of polymer.

FIG. 5 is a graph showing crystal growth rate of ritonavir as a function of ritonavir supersaturation with the y-axis being a ratio of the growth rate of ritonavir in the absence of polymer to the growth rate of ritonavir in the presence of polymer at each supersaturation.

FIG. 6 is a graph of the crystal growth rate of ritonavir at an initial solution concentration of 10 μg/mL in the presence of cellulose derivatives (5 μg/mL), with the data arranged in order of hydrophobicity: least hydrophobic to most hydrophobic (left to right).

FIG. 7 is a graph of the crystal growth rate of ritonavir at an initial solution concentration of 10 μg/mL in the presence of novel cellulose derivatives (5 μg/mL), with the polymers arranged in order of degree of substitution of the adipate group: high to low DS (Adp) from left to right.

FIG. 8 is a graph of the crystal growth rate of ritonavir at a pH of 3.8 and 6.8 with an initial ritonavir solution concentration of 10 μg/mL and polymer concentration of 5 μg/mL.

FIG. 9 is a graph showing dissolution of amorphous solid dispersions of ritonavir, where a binary combination of PVP and CAP 504-0.2 Adp is able to maintain solution concentration close to the amorphous solubility of ritonavir.

FIGS. 10A-C are SEM micrographs of ritonavir seed crystals: (A) before and (B) after crystal growth experiment in the absence of polymer at an initial concentration of 10 mg mL⁻¹ (σ=2.0) and (C) after crystal growth experiment in the absence of polymer at an initial concentration of 20 mg mL⁻¹.

FIGS. 11A-D are graphs showing dissolution from: (A) ellagic acid (EA), EA/PVP 1/9 physical mixture, EA/polymer 1/9 solid dispersions (pH 6.8, UV-vis); (B) EA/polymer 1/3 solid dispersions (pH 6.8, UV-vis); (C) EA/CAAdP (1/3, 1/9) co-precipitating solid dispersions (CPSD) and EA/CAAdP/PVP (1/4.5/4.5) evaporation solid dispersion (EVSD), after centrifugation at 14,000×g for 10 min (pH 6.8, UV-vis); and (D) dissolution of EA and EA/polymer 1/9 ASDs (pH 1.2, UV-vis).

FIG. 12 is a graph showing highest percentage of resveratrol in the polymer-resveratrol amorphous solid dispersion (prepared by rotary evaporation using 1:1 (by weight) dichloromethane-ethanol as a solvent) that creates an X-ray amorphous dispersion.

FIG. 13 is a graph showing crystal growth rate effectiveness ratio of ritonavir in the presence of individual polymers and their combinations (1:1 ratio) at an initial ritonavir concentration of 10 μg/mL.

FIGS. 14-16 are graphs showing induction times for respectively ritonavir, efavirenz, and celecoxib from unseeded desupersaturation experiments, in the absence and presence of polymers.

FIGS. 17-19 are graphs showing a comparison of the growth rate ratio of celecoxib, efavirenz and ritonavir, respectively, at an initial concentration of 10 μg/mL in the absence of polymer (R_{g}_{ₒ}) to the growth rate in the presence of polymer (R_{g}_{ₚ}).

FIGS. 20A-E are graphs showing particles size change and/or agglomeration of ritonavir in solution with various polymers over time.

FIG. 20F is a graph showing the zeta potential for CAP Adp at pH 6.8.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

Polymers of the invention are useful for increasing the aqueous solution concentration of compounds and/or for stabilizing such solutions. Such polymers can be selected such that the polymer is preferably not absorbed by the body and is preferably not toxic, including its chemical or enzymatic breakdown by-products, if any. Additionally, the chemical structure of the polymer is such that polymer-drug interactions (for example, CO₂H—:NR₃) are maximized and the polymer retains the ability to disperse in water.

Functions of the polymer include that it may be capable of stabilizing the drug in supersaturated aqueous solution and in the solid phase and minimize, prolong the onset of, or avoid or prevent crystallization of the drug. Another preferred characteristic of the polymer is that it may have a high Tg to immobilize drug against crystallization, even in the presence of high humidity (since water may act as a plasticizer, lowering the effective Tg) and high ambient temperature (for example up to 50-60° C.), and preferably for years. In embodiments, the polymer can also be amorphous. Release properties of the drug in combination with such polymers can include pH control, and slow release (ideally zero order, permitting once a day dosage or even less frequent dosage).

Desired polymer performance characteristics for polymers and compositions of the invention can include any one or more of: 1) the ability to stabilize the drug against crystallization in the solid phase similar solubility parameter to that of drug, specific polymer-drug interactions, high glass transition temperature (T_{g})); 2) the ability to stabilize the drug in solution after release but prior to absorption from the GI tract (at least slight (μg/mL) polymer solubility in pH 6.8 buffer, plus affinity for drug as in 1)); 3) the desired drug release profile (release rate will decline as polymer hydrophobicity rises, groups ionizable at neutral pH (e.g., —CO₇H) can provide release trigger). Properly designed carboxylated polysaccharide derivatives are excellent candidates for amorphous dispersion polymers, since as a class they tend to have low toxicity and high T_{g} values. A high T_{g} helps maintain the matrix in the glassy state at high humidity and relatively high ambient temperatures, in order to limit molecular motion of drug molecules and thus inhibit drug crystallization in storage and transport.

Exemplary compositions according to embodiments of the invention can comprise: at least one amorphous drug with a solubility of less than about 1 mg/mL; at least one first polymer chosen from cellulose esters of formula I:

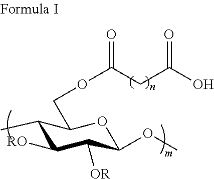

wherein n of the ω-carboxyalkanoyl group,

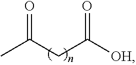

is 1, 2, 3, 4, 5, 6, 7, 8, 9, or 10; and

wherein R is chosen from: a hydrogen atom; and an alkanoyl group; and

wherein the number of repeating units of the polymer, m, ranges from 1 to 1,000,000, such as from 10 to 100,000, or from 100 to 1,000, such as 1-6,000.

Such compositions can have polymers with a degree of substitution with respect to the ω-carboxyalkanoyl group

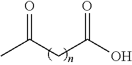

of 0.05-2. In preferred embodiments the degree of substitution the ω-carboxyalkanoyl group can be 1 or near 1. Indeed, it has been found that polymers that are especially effective are those with a degree of substitution of the ω-carboxyalkanoyl groups of between 0.5-1. Such compositions can have a total degree of substitution of the alkanoyl group and the ω-carboxyalkanoyl group of at least 2.0.

In embodiments, compositions can comprise polymers wherein the alkanoyl group is chosen from at least one of acetyl, propionyl, butyryl, valeroyl, hexanoyl, nonanoyl, decanoyl, lauroyl, palmitoyl, or stearoyl groups. In embodiments, compositions can comprise polymers wherein the ω-carboxyalkanoyl group is chosen from at least one of succinoyl, glutaroyl, adipoyl, sebacyl, and suberyl groups.

Compositions of the present invention can generate drug solution concentrations higher than the solubility of the drug. This is preferably true for the drug in buffer solutions that simulate small intestine contents. Especially preferred are compositions wherein drug solution concentration generated by the composition is higher than the solubility of the drug in pH 6.8 buffer solutions.

Preferred compositions comprise those wherein bioavailability of the drug is enhanced above that of the drug by itself.

Exemplary compositions of the invention can further comprise a second polymer. The second polymer can be selected such that it enhances or retards release rate of the drug, and/or enhances solution concentration generated from the composition. In embodiments, the compositions can comprise a second polymer that is more water-soluble than the first polymer. The second polymer can be chosen from at least one of poly(vinylpyrrolidinone), HPMC, poly(ethylene glycol), and poly(propylene glycol).

Preferred compositions of the invention are formulated such that the drug is amorphous for at least 1 year. Especially preferred are compositions wherein the drug is amorphous for at least 4 years.

Upon dispersion in buffer solutions that simulate the small intestine, the drug of preferred compositions dissolves to a maximum concentration, and at least 90% of that concentration is maintained for at least 24 h. Preferably, in such compositions the buffer solution is a phosphate buffer with pH 6.8.

Preferred are compositions wherein the drug is chosen from drugs having a solubility of less than 1 mg/mL. Especially preferred are compositions wherein the drug is chosen from at least one of ritonavir, efavirenz, etravirine, celecoxib, and clarithromycin. Such compositions may be useful in the treatment of HIV and/or AIDS. A specific mechanism of action for such drugs may include activity as protease inhibitors and/or activity in inhibiting metabolism of protease inhibitors to increase efficacy of other protease inhibitors used in combination with the drug. In other preferred compositions, the drug may be chosen from at least one of curcumin, ellagic acid, quercetin, naringenin, and resveratrol. Such compositions may be useful in the treatment of cancer and/or as antioxidants.

The drug in preferred compositions can be chosen from any drug where it is desirable to increase solubility or stability of the drug in solution. Particular examples of drugs that can be used in embodiments of the compositions of the invention include one or more of antihypertensives, antianxiety agents, anticlotting agents, anticonvulsants, blood glucose lowering agents, decongestants, antihistamines, antitussives, antineoplastics, beta blockers, anti-inflammatories, antipsychotic agents, cognitive enhancers, cholesterol reducing agents, triglyceride reducing agents, anti-atherosclerotic agents, anti-obesity agents, autoimmune disorder agents, anti-impotence agents, anti-bacterial and anti-fungal agents, hypnotic agents, anti-Parkinsonism agents, anti-Alzheimer's disease agents, antibiotics, antidepressants, antiviral agents, glycogen phosphorylase inhibitors, protease inhibitors, and cholesteryl ester transfer protein inhibitors.

Polymers include and compositions of the invention can comprise any commercially available cellulose polymer or polymers as the first or second polymer, including but not limited to carboxylated cellulose derivatives. Particular polymers include but are not limited to hydroxypropyl methylcellulose (HPMC), hydroxypropyl methylcellulose acetate succinate (HPMCAS), carboxymethyl cellulose acetate butyrate (CMCAB), poly(vinyl pyrollidinone) (PVP), poly(vinyl pyrollidinone-vinyl acetate) (PVP/VA). Preferred compositions can alternatively or additionally comprise a novel synthesized polymer of the invention, for example, cellulose acetate adipate propionate (CAAdP). CAPH (cellulose acetate phthalate), HPMCPH (hydroxypropylmethylcellulose phthalate), CASub (cellulose acetate suberate), CASeb (cellulose acetate sebacate), and PEG (polyethylene glycol) can also be used. Compositions with cellulose-carboxyalkanoates of longer chain length (e.g. adipates, suberates, sebacates) have enhanced hydrolytic stability as compared with succinate and glutarate. Further, any of the polymers listed in Table 1 or 2 can be used in compositions of the invention.

According to embodiments, the cellulose ester polymers can be prepared from carbohydrate, oligosaccharide, or polysaccharide esters such as cellulose with a weight average molecular weight (MW) ranging from about 162 to 1,000,000 as measured by GPC (gel permeation chromatography) with polystyrene equivalents, mass spectrometry, or other appropriate methods. In embodiments, the number-average molecular weight (Mₙ) of polymers of the invention can range from about 3,000 to about 75,000, such as from about 9,000 to about 62,000, or from about 16,000 to about 50,000, and so on. Further, the degree of polymerization of the polymers in embodiments can range from 1 to 10,000, such as from 50 to 500, or from 500 to 5,000, or from 1,000 to 3,000.

The chain length or degree of polymerization (DP) can have an effect on the properties of oligosaccharide and polysaccharide derivatives. In the context of this specification, the degree of polymerization is the number of anhydroglucose units in the polymer molecule. Substituted oligosaccharide or polysaccharide derivatives of embodiments of the present invention include polymers comprising from 2 (e.g., cellobiose) to about 10,000 anhydroglucose repeating units (AGU). Preferred esters of embodiments of the invention, such as cellulose esters, comprise from 5 to 10,000 AHG repeating units, such as from 10 to 8,000, or from 15 to 7,000, or from 20 to 6,000, or from 25 to 4,000, or from 30 to 3,000, or from 50 to 1,000, or from 75 to 500, or from 80 to 650, or from 95 to 1,200, or from 250 to 2,000, or from 350 to 2,700, or from 400 to 2,200, or from 90 to 300, or from 100 to 200, or from 40 to 450, or from 35 to 750, or from 60 to 1,500, or from 70 to 2,500, or from 110 to 3,500, or from 150 to 2,700, or from 2,800 to 5,000, and so on.

The extent and manner in which hydroxyl groups of the carbohydrate starting material are derivatized can be described by the degree of substitution (DS). The term “degree of substitution” can refer to the average total number of substituents, such as acyl (alkanoyl) and/or ω-carboxyalkanoyl groups, per anhydroglucose ring of the cellulose molecule, or said another way can refer to the average number of hydroxyl positions on the anhydroglucose unit of the carbohydrate that have been reacted. Since each anhydroglucose unit has three hydroxyl groups, the maximum value for DS is three (ignoring the possibility of substitution on the end groups, the terminal 1-OH and 4-OH groups). According to embodiments of the invention, the polymer esters can have a degree of substitution ranging anywhere from above 0 to 3. In preferred embodiments, the degree of substitution of the ω-carboxyalkanoyl group on the polymer is at least 0.05, such as from 0.05 to 1, or from 0.07 to 0.9, or from 0.09 to 0.8, or from 0.1 to 0.7, or from 0.2 to 0.6, or from 0.3 to 0.5, or from 0.4 to 1.2, or from 1.3 to 3, or from 1.4 to 2.9, or from 1.5 to 2.5, or about 2. Additionally, or alternatively, the total degree of substitution of the alkanoyl group and the ω-carboxyalkanoyl group of the polymer in preferred embodiments is at least 2.0. The total degree of substitution of the alkanoyl group and the ω-carboxyalkanoyl group can range from 0 to 3, such as from 0.05 to 2.85, or from 1.05 to 2.55, or from 1.1 to 2.4, or from 1.15 to 2.25, or from 2 to 3, such as from 2.05 to 2.95, or from 2.1 to 2.8, or from 2.2 to 2.75, or from 2.25 to 2.7, or from 2.3 to 2.65, or from 2.35 to 2.45, and so on. In embodiments, the degree of substitution of the alkanoyl group or groups can be determined by subtracting the degree of substitution of the ω-carboxyalkanoyl group from the total degree of substitution. In preferred embodiments, the total degree of substitution ranges from 2 to 3, such as from 2.16 to 2.98, or from about 2.3 to about 2.9, or from about 2.35 to about 2.84, or from about 2.46 to 2.79, or from about 2.49 to about 2.89 and so on.

The degree of substitution of the cellulose esters can be determined according to conventional techniques, such as by proton nuclear magnetic resonance (NMR). In embodiments, for example, the degree of substitution (DS) can be determined from NMR spectra acquired on an INOVA 400 spectrometer operating at 400 MHz. The sample tube size can be 5 mm, and the sample concentrations can be about 10 mg mL⁻¹ in CDCl₃ or DMSO-d₆. Substituent DS can be calculated from the proton NMR spectra using the ratios of the integrals for appropriate acyl protons to the backbone AGU protons.

### Example I

### Ritonavir Compositions

Large crystal lattice energy/high melting point and, high and positive octanol-water partition coefficient (Log P) are some of the underlining factors causing poor aqueous solubility. To demonstrate the effectiveness of various polymers, a model drug compound with low aqueous solubility, ritonavir, an HIV protease inhibitor, was tested. The drug-polymer systems of embodiments of the invention enable the inhibition of the nucleation and crystal growth stages of crystallization from supersaturated solutions. Stable solutions can be obtained using drug compounds having slow crystallization tendency, such as ritonavir, in combination with polymers, including for example cellulose derivatives provided herein, which are characterized by a wide range of substitution groups and different degrees of substitution.

Ritonavir, a general chemical structure for which is shown below, was purchased from Attix Corporation, Toronto, Ontario, Canada:

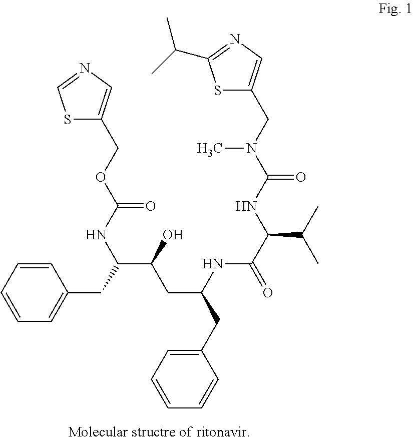

The commercially available polymers were purchased from various sources: poly (vinyl pyrrolidinone) K29/32 (PVP), polyacrylic acid and cellulose acetate phthalate (Sigma-Aldrich Co., St. Louis Mo.), Kollidone® VA 64 (PVP/VA K28) (BASF, Germany) (poly (vinyl pyrrolidinone-vinyl acetate)), hydroxypropyl methylcellulose (HPMC) (606 grade) and hydroxypropyl methylcellulose acetate succinate (HPMCAS) (AS-MF grade) (Shin-Etsu Chemical Co., Ltd. (Tokyo, Japan)), poly(allylamine), poly(N-methylvinylamine), polyethylenimine, poly(4-vinylphenol), poly(N-iso-propylacrylamide) and poly(4-vinylpyridine N-oxide) (Polysciences, Inc. Warrington, Pa.), poly(N,N-dimethyl acrylamide), poly(vinylacetate), poly(vinyl alcohol), poly(4-vinylpyridine), polyacrylamide (Scientific Polymer Products, Inc., Ontario, N.Y.), Eudragit L100 (Degussa (Rohm GmbH & Co. KG, Germany), hydroxypropylcellulose (Hercules Polymer and Chemicals, Inc. Florida). Carboxymethylcellulose acetate butyrate was obtained from Eastman Chemical Company, Kingsport, Tenn.

Abbreviations for novel polymers of the invention and commercially available polymers that can be used according to embodiments of the invention are provided in Table 1 and Table 2. Polymers of the invention may be referred to in this specification by one or more names, which one of skill in the art would readily recognize especially in consideration of additional identifying information provided about the polymers in this specification, such as degree of substitution. For example:

cellulose propionate adipate may be referred to as CP Adp;

cellulose acetate 320S adipate, CA 320S Adp, and CA Adp 0.67 are synonymous;

cellulose acetate propionate adipate 3X is CAP Adp 3X or CAP Adp 0.85;

cellulose acetate 398-30 adipate is CA 398-30 Adp or CA Adp 0.21;

cellulose acetate butyrate adipate 3X is CAB Adp 30 or CAB Adp 0.81;

cellulose acetate propionate 504.02 adipate is CAP 504-0.2 Adp, or CAP Adp 1X, or may also be referred to as CAP Adp 0.33;

cellulose acetate propionate sebacate 3X is CAP Seb 3X or CAP Seb 0.67;

cellulose acetate propionate 482-20 adipate is CAP 482-20 Adp or CAP Adp 0.19;

cellulose acetate propionate suberate is CAP Sub or CAP Sub 0.26;

cellulose acetate butyrate 381-30 adipate is CAB 381-30 Adp or CAB Adp 0.19;

cellulose acetate butyrate 553-0.4 adipate is CAB 553-0.4 Adp or CAB Adp 1X, or may also be referred to as CAB Adp 0.25;

cellulose acetate propionate sebacate is also CAP Seb or CAP Seb 0.24;

cellulose acetate butyrate suberate is CAB Sub or CAB Sub 0.25; and

cellulose acetate butyrate sebacate is CAB Seb or CAB Seb 0.22.

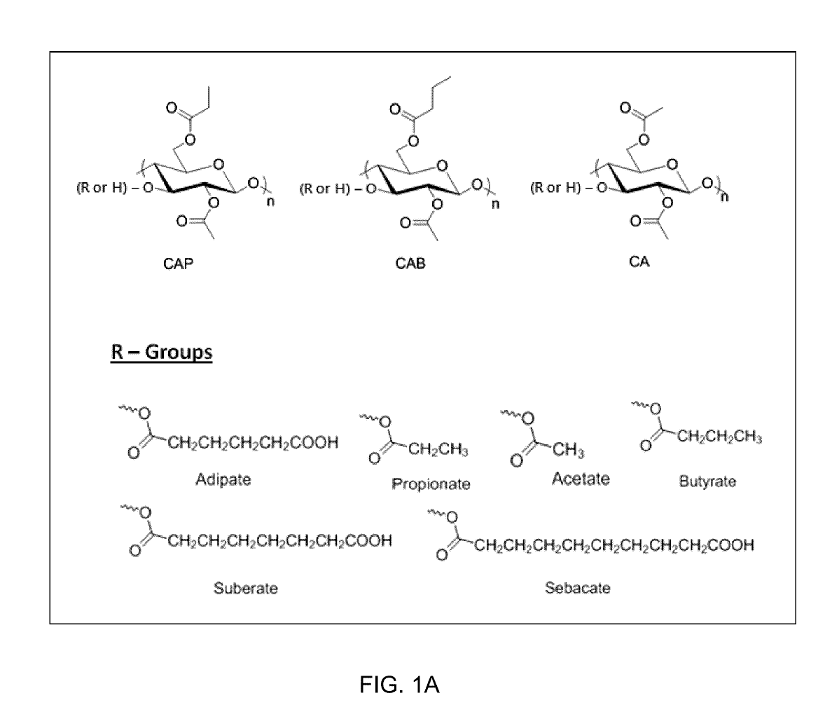

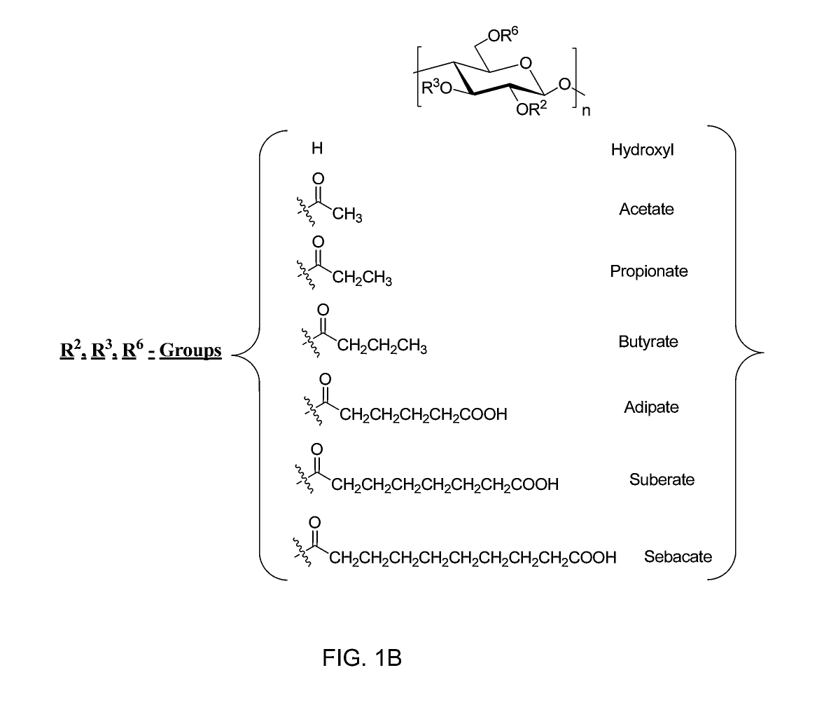

A general synthetic scheme for the novel polymers is presented in Scheme 1. FIG. 1 shows the molecular structure of representative novel synthesized polymer derivatives.

More particularly, FIGS. 1A-B are schematic diagrams of the molecular structure of representative cellulose derivatives of embodiments of the present invention, including various substituents that can be incorporated into the polymers. It is noted that the structures illustrated in FIGS. 1A-B are not meant to imply specific locations (O-2, O-3, and/or O-6) for each substituent. Indeed, substituents are presumed to be distributed relatively randomly.

Polymers of the invention can include cellulose esters of Formula I:

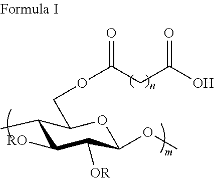

wherein n of the ω-carboxyalkanoyl group,

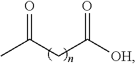

is 1, 2, 3, 4, 5, 6, 7, 8, 9, or 10; and wherein R is chosen from: a hydrogen atom; and an alkanoyl group; and wherein m repeating units of the polymer range from 1 to 1,000,000, such as from 10 to 100,000, or from 100 to 1,000, or from 1 to 6,000.

Theoretical Amorphous Solubility with Moisture Sorption Effect.

Amorphous solubility can be approximated by estimating the free energy difference between crystalline and amorphous forms using the Hoffman equation:

$$Δ {G}_{a - c} = \frac{Δ {H}_{fus} ( {T}_{m} - T ) T}{\text{?}} \\ \text{?} \text{indicates text missing or illegible when filed} \qquad ( Equation 1 )$$

The ratio of amorphous and crystalline solubility (Sₐₘₒᵣₚₕₒᵤₛ/S_{crystalline}) is then estimated from the free energy difference (ΔG_{a→σ}) calculated from heat of fusion (ΔH_{f}) and melting temperature (Tₘ) of the crystalline material determined from DSC analysis, as well as the experimentally determined crystalline solubility:

$$\frac{{S}_{amorphous}}{{S}_{crystalline}} \approx \frac{{a}_{amorphous}}{{a}_{crystalline}} exp [ \frac{Δ {G}_{a}}{RT} ] \qquad ( Equation 2 )$$

In addition, the quantitative approach developed by Murdande et al. can be used to determine the impact of water in the amorphous solid on the thermodynamic activity and hence estimated solubility advantage. See Murdande, S. B., Pikal, M. J. Shanker, R. M. & Bogner, R. H., “Solubility Advantage of amorphous pharmaceuticals: I. A thermodynamic analysis,” J. Pharm. Sci., 99(3): 1254-1264, (2009) (“Murdande I”) and Murdande, S. B., Pikal, M. J. Shanker, R. M. & Bogner, R. H., “Solubility Advantage of amorphous pharmaceuticals: II. Application of quantitative thermodynamic relationships for prediction of solubility enhancement in structurally diverse insoluble pharmaceuticals,” Pharm. Res. 2010 December, 27(12):2704-14. (“Murdande II”). This method involves determining the number of moles of water absorbed per mole of solute as a function of relative humidity and estimating the water content at a relative humidity of 100 (water activity of 1). The activity of the amorphous solute is estimated by applying the Gibbs-Duhem equation to water sorption isotherm data for the amorphous solid. The detailed thermodynamic analysis is in the literature. See Murdande I.

The final form of the Gibbs-Duhem equation, I(a₂), may be written as:

$$I ( {a}_{2} ) = {( \frac{{n}_{1}}{{n}_{2}} )}_{Henry} + {\int }_{{a}_{2}^{H}}^{{a}_{1}^{S}} ( \frac{{n}_{1}}{{n}_{2}} ) \frac{1}{{a}_{2}} d {a}_{1} \qquad ( Equation 3 )$$

where n₁ and n₂ are moles of water and solute, respectively, ā₁^{H} is the activity of water at the Henry's law limit and ā₁^{g} is the activity of water in the solid in equilibrium with dilute solution (ā₁^{g} is the extrapolated water activity at unit water activity). Equation 3 can be divided into two parts, the Henry's law region, which ranges from zero water content to the limit of linearity of water activity and a “high water” region. The first term,

$${( \frac{{n}_{1}}{{n}_{2}} )}_{Henry} ,$$

is the concentration value at the limit, while the integral in the “high water” region may be evaluated numerically (by the summation of the products,

$${〈 ( \frac{{n}_{1}}{{n}_{2}} ) \frac{1}{{a}_{1}} 〉}_{i + 1 , t} ( {a}_{1}^{i + 1} - {a}_{1}^{t} ) ) )$$

using the water sorption isotherm data. See Murdande I.

Equation 4 represents the incorporation of water sorption effects into the thermodynamic prediction of solubility:

$$\frac{{S}_{amorphous}}{{S}_{crystalline}} = exp [ - l ( {a}_{2} ) ] \cdot exp [ \frac{Δ {G}_{a}}{RT} ] \qquad ( Equation 4 )$$

Moisture Sorption.

The moisture sorption profile of amorphous ritonavir was determined at 37° C. using a TA Q5000 dynamic vapor sorption analyzer (TA Instruments, New Castle, Del.) equipped with a humidity controlled chamber and an ultrasensitive thermobalance. Approximately 15 to 25 mg of sample was placed into the sample pan and dried at 37° C. and 0% humidity until the weight change was less than 0.01 wt % over 5 minutes. Subsequently, the moisture sorption isotherm was measured by equilibrating the sample under controlled relative humidity (RH) ranging from 5% RH to 95% RH, in 10% RH intervals. Equilibrium was assumed to be attained when the weight change was less than 0.01 wt % over 5 minutes at each RH step. Moisture sorption isotherms were obtained on three individually prepared samples of amorphous ritonavir.

Solubility Studies.

The equilibrium solubility of ritonavir was determined in the absence and presence of selected polymers, at a polymer concentration of 5 μg/mL. An excess amount of ritonavir was equilibrated in sodium phosphate buffer, pH 6.8, at 37° C. for 48 hours. The supernatant was separated from excess solid in solution by ultracentrifugation at 40,000 RPM (equivalent of 274,356×g) in an Optima L-100 XP ultracentrifuge equipped with Swinging-Bucket Rotor SW 41 Ti (Beckman Coulter, Inc., Brea, Calif.). Subsequently, the supernatant was diluted and solution concentration was determined using an Agilent 1100 high performance liquid chromatography (HPLC) system (Agilent Technologies, Santa Clara, Calif.). The chromatographic separation was performed with a Zobrax SB-C18 analytical column (150×2.1 mm I.D., 5 μm, 100 Å) (Agilent Technologies, Santa Clara, Calif.). Ritonavir was detected by UV absorbance detection at a wavelength of 240 nm. The mobile phase used consisted of 10 mM sodium phosphate buffer, pH 6.8 and acetonitrile and mobile phase flow was maintained 0.2 mL/min. The total analytical run time was 20 minutes. The injection volume was 20 μl.

In general, polymer concentration for compositions of the invention can range from 0-10 mg/mL, such as from about 1 μg/mL to about 1 mg/mL. Preferred polymer concentrations range from 15-500 μg/mL, such as from 100-250 μg/mL. Especially preferred polymer concentrations according to embodiment of the invention range from 0-50 μg/mL, such as from 2-40 μg/mL, such as from 7-30 μg/mL, or from 10-25 μg/mL, or even 15-22 μg/mL, and more preferably from 12-20 μg/mL. Preferred compositions comprise polymer in a concentration of about less than 35 μg/mL.

Nucleation—Induction Time.

The period between the generation of supersaturation and initiation of nucleation was determined by monitoring the absorbance at 280 nm using an ultraviolet/visible spectrometer, fiber optic coupled with a dip probe (SI Photonics® Tucson, Ariz.). Wavelength scans were acquired every 45 seconds. Precipitation was characterized by an increased extinction at 280 nm wavelength. A supersaturated solution of ritonavir was generated by adding 0.4 mL of ritonavir dissolved in methanol at a concentration 4 mg/mL, to 80 mL sodium phosphate buffer, pH 6.8, at 37° C., providing an initial solution concentration of ritonavir of 20 μg/mL. The drug of compositions of the invention can be present in any amount ranging from about 0-100 μg/mL, such as from about 5-10 μg/mL, or from 15-20 μg/mL, or from 25-30 μg/mL, or from 35-50 μg/mL, or from 55-75 μg/mL, or from 80-90 μg/mL. Depending on the drug compound and the types and amounts of polymers used, the most preferred concentration of drug in the compositions ranges from about 5-25 μg/mL. A syringe pump (Harvard Apparatus, Holliston, Mass.) was used to control the rate of addition of organic solution of ritonavir to buffer solution; the flow rate was 0.20 mL/min. A Corning stir plate was used to stir the solution at a speed of 300 rpm. The ability of the various polymers to affect the nucleation of ritonavir was evaluated using a polymer concentration of 5 μg/mL. All experiments were performed in triplicate.

Crystal Growth Rate.

Crystal growth rate was characterized by measuring the rate of desupersaturation in the presence of seed crystals. The rate of desupersaturation of ritonavir in a precipitating solution is found in the literature. See Sohnel, O. and Mullin, J. W., “Precipitation of calcium carbonate,” Journal of Crystal Growth, 60: 239-250 (1982).

The equation can be written as:

$$- \frac{d [ C ]}{d t} = {K}_{c} A ( t ) {( [ C ] - {[ C ]}_{eq} )}^{g} \qquad ( Equation 5 )$$

where K_{Q} is the desupersaturation rate constant, A(t) is the crystal surface area, C is ritonavir concentration at time, t, [C′]_{eq} is the equilibrium solution concentration of ritonavir and g is the growth rate order. The crystal growth rate may be expressed by:

$$\frac{d r}{d t} = {{K}_{g} ( [ C ] - {[ C ]}_{eq} )}^{g} \qquad ( Equation 6 )$$

K_{g}, the growth rate constant, is related to K_{ç}, in Equation 5, by:

$${K}_{g} = \frac{{K}_{g} M}{ρ} \qquad ( Equation 7 )$$

where M and ρ are molecular weight and density of the crystals, respectively.

Crystal growth rate experiments were performed in the absence and presence of pre-dissolved polymers and an initial ritonavir concentration of 10 μg/mL. Additional experiments were conducted at initial ritonavir concentrations of 5 and 20 μg/mL in the presence of pre-dissolved PVPVA, CMCAB and CAP Adp 3X to investigate the influence of supersaturation on growth rate. A polymer concentration of 5 μg/mL was used for all experiments and all were performed in triplicate. Seed crystals were characterized using cross polarized optical microscopy, Nikon Eclipse E600 Pol microscope, with NIS-Elements version 2.3 software package (Nikon Co., Tokyo, Japan). A total of 500 needle-shaped seed crystals were counted. The average needle length was 2.15 p.m. The seeds were added to the crystallization media and allowed to equilibrate at 37° C. prior to addition of solubilized ritonavir. Data collection began immediately after ritonavir pre-dissolved in methanol was added to the medium. An overhead stirrer was used to stir the solution at a speed of 400 rpm. The slope of the concentration vs. time curve over the first 2 minutes of the experiment was taken as the initial crystal growth rate. In addition, the effect of ionizable groups on crystal growth rate was evaluated by performing the experiments at different pH conditions, pH 3.8 and 6.8, using 100 mM sodium acetate and sodium phosphate buffer, respectively.

Solubility Parameter.

The solubility parameter was used to characterize the relative hydrophobicity of the novel polymers. The method proposed by Fedors, R. F. was used to estimate the solubility parameter. See Fedors, R. F., “A method for estimating both the solubility parameter and molar volumes of liquids,” Polymer Engineering and Science, 14 (2): 147-154 (1974). This method requires only knowledge of the structural formula of the compound. It is based on group additive constants and the contribution of a large number of functional groups was evaluated. Solubility parameter can be evaluated using:

$$δ = \sqrt{\frac{{\sum }_{i} Δ {e}_{i}}{{\sum }_{i} Δ {v}_{i}}} = \sqrt{\frac{Δ {E}_{V}}{V}} \qquad ( Equation 8 )$$

where the Δeₜ and Δvₜ are the additive atomic and group contribution for the energy of vaporization and molar volume respectively. The contributions applicable at a temperature of 25° C. are presented in reference. See Fedors (1974). For high molecular weight polymers that have a glass transition greater than 25° C., there is a deviation between the experimentally measured ΔEᵥ and V and the estimated values. A small correction factor was introduced to take into account the divergence in the V values:

Δvₜ=4n,n<3 (Equation 9)

Δvₜ=2n,n≧3 (Equation 10)

where n is the number of main chain skeletal atoms in the smallest repeating unit of the polymer. The solubility parameter of the novel synthesized cellulose derivatives is presented in Table 2.

Preparation of Amorphous Solids.

Amorphous samples of ritonavir were prepared by the solvent evaporation method, specifically, spin coating. Spin-coating was done using KW-4A spin-coater (Chemat Technology, Inc., Northridge, Calif.). 30 mg of crystalline ritonavir was dissolved in 0.5 mL of methanol. Amorphous solid dispersions of ritonavir were prepared by dissolving a total of 60 mg of solid material in the desired ratio of polymer and drug in 1.0 mL methanol. Two to three drops of solution were placed on an 18 mm diameter circular glass slide (VWR International, LLC) and spin-coated. Residual solvents were removed by drying the amorphous films under vacuum at room temperature for 24 hours. The glass slides were weighed before and after spin coating (after drying) to determine the amount of solid in the amorphous film. The amorphous films were stored in desiccators containing Drierite® at room temperature until analyzed. Prior to use, the spin coated films were analyzed by cross-polarized light microscopy to verify their amorphous nature.

Dissolution of Amorphous Solids.

Dissolution experiments for amorphous ritonavir were performed in a jacketed flask connected to a circulating water bath maintained at 37° C. Amorphous solids prepared using the above mentioned method were used. The glass slide was placed on a star-shaped stir bar (VWR International, LLC) and a stir plate was used to stir the solution at a speed of 300 rpm. The dissolution medium used was 100 mM sodium phosphate buffer, pH 6.8. Solution concentration was determined by monitoring absorbance at 240 nm using a SI photonics UV-Vis fiber optic single probe system (path-length 5 mm). Wavelength scans (200-450 nm) were performed every 45 seconds. Second derivatives of the spectra were taken for the calibration set as well as the sample data in order to alleviate particle scattering effects. Calibration solutions were prepared in methanol. The aforementioned dissolution setup is similar to the rotating disk method used for determination of intrinsic dissolution rate.

TABLE 1
Commercially Available Polymers for use in the Compositions

| Polymer | Abbreviation |
|---|---|
| Poly vinylpyrrolidone K 29/32 | PVP |
| Poly vinylpyrrolidone vinyl acetate K 28 | PVPVA |
| Poly(allylamine) | PAlAmn |
| Poly(N-methylvinylamine) | Pn-MVAmn |
| Polyethylenimine, Linear | PEE |
| Poly(N,N-dimethyl acrylamide) | Pnn-DMAAmd |
| Poly(vinylacetate) | PVAc |
| Poly(vinyl alcohol), 99.7% hydrolyzed | PVAh |
| Poly(4-vinylpyridine), linear | PVPd |
| Polyacrylamide | PAcAmd |
| Polyacrylic acid | PAA |
| Poly(4-vinylphenol) | PVPh |
| Poly(4-vinylpyridine N-oxide) | PVPdn-O |
| Eudragit L100 | EUD L100 |
| Hydroxypropyl Cellulose | HPC |
| Cellulose Acetate Phthalate | CAPh |
| Hydroxypropyl methyl cellulose AS-MF | HPMC |
| Hydroxypropyl methyl cellulose acetate succinate | HPMCAS |
| Carboxymethyl cellulose acetate butyrate | CMCAB |

Preferred cellulose esters, such as carboxylated cellulose esters, that can be used according to embodiments of the invention include but are not limited to cellulose acetate adipate, cellulose acetate propionate, cellulose acetate butyrate, cellulose acetate sebacate, cellulose acetate suberate, cellulose acetate adipate propionate, cellulose acetate adipate butyrate, cellulose acetate adipate suberate, cellulose acetate adipate sebacate, cellulose acetate propionate suberate, cellulose acetate propionate sebacate, cellulose acetate propionate butyrate, cellulose acetate butyrate suberate, cellulose acetate butyrate sebacate, carboxymethylcellulose acetate butyrate, carboxymethylcellulose acetate propionate, hydroxymethylcellulose acetate succinate, and cellulose propionate adipate to name a few.

Preferred are cellulose esters that are relatively hydrophobic polymers, which aids their interaction with hydrophobic drug compounds and stabilizes the amorphous drug. It also slows the penetration of water into the matrix, and of aqueous drug solution back out of the matrix, thereby promoting desirable slow drug release. The carboxyl groups not only provide specific interactions with the drug molecule to enhance stability of the amorphous dispersion, but they provide the mechanism for drug release. Ionization of the carboxyl groups in the neutral pH of the small intestine causes swelling and/or dissolution of the carboxylated cellulose ester matrix, permitting an infusion of water into the matrix and thus drug dissolution.

Solubility enhancements for compounds, such as flavonoids from such matrices, have been observed in for example in International Patent Application No. PCT/US11/56946 entitled, “CELLULOSE DERIVATIVES FOR ENHANCING BIOAVAILABILITY OF FLAVONOIDS,” which is incorporated by reference herein in its entirety. It has been found that it may be desired to stabilize the aqueous flavonoid solution, after dissolution of the flavonoid from the carboxylated cellulose ester matrix, against flavonoid recrystallization from the resulting supersaturated flavonoid solution. Ellagic acid, for example, is poorly soluble in organic solvents like acetone/ethanol. Addition of carboxymethylcellulose acetate butyrate to acetone/ethanol permitted dissolution of ellagic acid in this solution, where in the absence of carboxymethylcellulose acetate butyrate, no dissolution occurred.

Although the water solubility of PVP can be an advantage, it can also lead to premature drug release and the enhanced solubility of PVP in acidic media means that PVP amorphous solid dispersions are likely to increase drug exposure to the gastric contents. It has also been found that PVP may be less effective than other polymer selections at preventing crystallization from solution. Although these characteristics do not eliminate PVP as a possible drug delivery vehicle, they may limit utility of PVP in some applications. PEG is also water soluble, but prone to crystallize. Again, PEG may be desirable in some compositions depending on the drug being solubilized, but perhaps not so desirable for other drugs. HPMCAS is somewhat hydrophobic, has a pH release trigger, and tends to provide relatively fast release of the drug due to its greater hydrophilicity (than CMCAB, for example).

TABLE 2
Abbreviation for novel synthesized cellulose derivatives and some
physical properties

| | | | | | | Solubility | Rank |
|---|---|---|---|---|---|---|---|
| | | DS | DS | DS | Rank | Parameter | (Solubility |
| Polymer | Abbreviation | (Adp) | (Other)* | (Total) | (DS (Adp)) | (MPa^{1/2}) | Parameter) |
| Cellulose | CP Adp | 0.48 | Pr 1.68 | 2.16 | 4 | 23.28 | 1 |
| Propionate | | | | | | | |
| Adipate | | | | | | | |
| Cellulose | CA 320S Adp | 0.67 | Ac 1.82 | 2.49 | 3 | 21.42 | 2 |
| Acetate | | | | | | | |
| 320S | | | | | | | |
| Adipate | | | | | | | |
| Cellulose | CAP Adp 3X | 0.85 | Ac 0.04; | 2.98 | 1 | 21.27 | 3 |
| Acetate | | | Pr 2.09 | | | | |
| Propionate | | | | | | | |
| Adipate 3X | | | | | | | |
| Cellulose | CA 398-30 | 0.21 | Ac 2.47 | 2.68 | 10 | 20.91 | 4 |
| Acetate | Adp | | | | | | |
| 398-30 | | | | | | | |
| Adipate | | | | | | | |
| Cellulose | CAB Adp 3X | 0.81 | Ac 0.14; | 2.84 | 2 | 20.86 | 5 |
| Acetate | | | Bu 1.99 | | | | |
| Butyrate | | | | | | | |
| Adipate 3X | | | | | | | |
| Cellulose | CAP 504-0.2 | 0.33 | Ac 0.04; | 2.46 | 5 | 20.56 | 6 |
| Acetate | Adp (CAP | | Pr 2.09 | | | | |
| Propionate | Adp 1X) | | | | | | |
| 504-0.2 | | | | | | | |
| Adipate | | | | | | | |
| Cellulose | CAP Seb 3X | 0.67 | Ac 0.04; | 2.80 | 3 | 20.41 | 7 |
| Acetate | | | Pr 2.09 | | | | |
| Propionate | | | | | | | |
| Sebacate | | | | | | | |
| 3X | | | | | | | |
| Cellulose | CAP 482-20 | 0.19 | Ac 0.10; | 2.79 | 11 | 20.29 | 8 |
| Acetate | Adp | | Pr 2.50 | | | | |
| Propionate | | | | | | | |
| 482-20 | | | | | | | |
| Adipate | | | | | | | |
| Cellulose | CAP Sub | 0.26 | Ac 0.04; | 2.39 | 6 | 20.19 | 9 |
| Acetate | | | Pr 2.09 | | | | |
| Propionate | | | | | | | |
| Suberate | | | | | | | |
| Cellulose | CAB 381-30 | 0.19 | Ac 1.0; | 2.89 | 10 | 20.11 | 10 |
| Acetate | Adp | | Bu 1.70 | | | | |
| Butyrate | | | | | | | |
| 381-30 | | | | | | | |
| Adipate | | | | | | | |
| Cellulose | CAB 553-0.4 | 0.25 | Ac 0.14; | 2.38 | 7 | 20.05 | 11 |
| Acetate | Adp (CAB | | Bu 1.99 | | | | |
| Butyrate | Adp 1X) | | | | | | |
| 553-0.4 | | | | | | | |
| Adipate | | | | | | | |
| Cellulose | CAP Seb | 0.24 | Ac 0.04; | 2.37 | 8 | 19.94 | 12 |
| Acetate | | | Pr 2.09 | | | | |
| Propionate | | | | | | | |
| Sebacate | | | | | | | |
| Cellulose | CAB Sub | 0.25 | Ac 0.14; | 2.38 | 7 | 19.84 | 13 |
| Acetate | | | Bu 1.99 | | | | |
| Butyrate | | | | | | | |
| Suberate | | | | | | | |
| Cellulose | CAB Seb | 0.22 | Ac 0.14; | 2.35 | 9 | 19.62 | 14 |
| Acetate | | | Bu 1.99 | | | | |
| Butyrate | | | | | | | |
| Sebacate | | | | | | | |

*Additional abbreviations: acetate (Ac), propionate (Pr) and butyrate (Bu).

Other properties of polymers of the invention are provided in Table 3.

TABLE 3
Number-Average Molecular Weight (Mₙ) and Glass Transition (T_{g})
Values for Novel Synthesized Cellulose Derivates

| polymer abbreviation | Mₙ^{a} (g/mol) | Tg (° C.) |
|---|---|---|
| CP Adp | 3850 | 90 |
| CA 320S Adp | 20500 | 134 |
| CAP Adp 0.85 | 9700 | 110 |
| CA 398-30 Adp | 26800 | 131 |
| CAB Adp 0.81 | 9500 | 82 |
| CAP Adp 0.33 | 12000 | 125 |
| CAP Seb 0.67 | 19100 | 74 |
| CAP 482-20 Adp | 58400 | 125 |
| CAP Sub | 16700 | 114 |
| CAB 381-30 Adp | 61000 | 115 |
| CAB Adp 0.25 | 18300 | 94 |
| CAP Seb 0.24 | 18800 | 116 |
| CAB Sub | 20900 | 90 |
| CAB Seb | 22600 | 83 |

^{a}Number-average molecular weight in polystyrene equivalents.

Scheme 1 below provides an exemplary general synthetic method for preparing select polymers of the invention, for example, ω-carboxyacyl derivatives of cellulose.

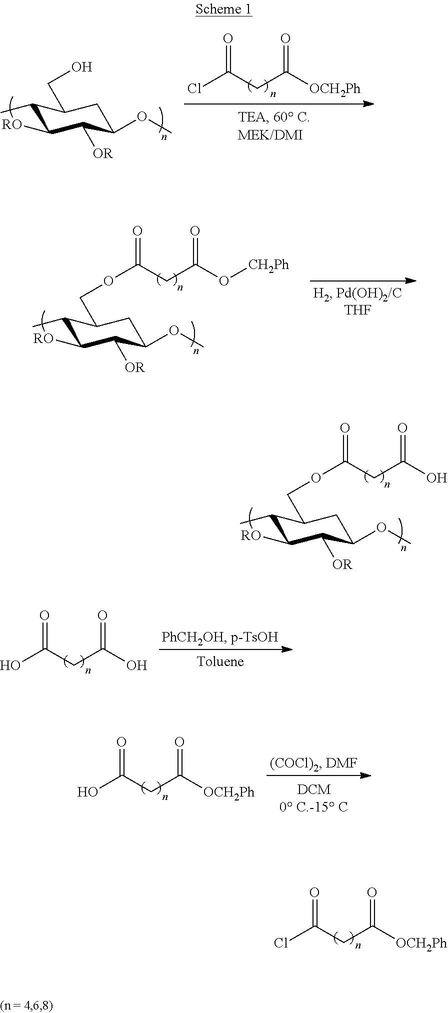

Substituent R as provided in Scheme 1 can be hydrogen, a COCH₃ group, or a COCH₂CH₃ group, or COCH₂CH₂CH₃ group. Note that the ester groups are relatively randomly distributed. Although it is possible, in embodiments, to prepare regioselectively substituted cellulose-carboxyalkanoates of the present invention, the cellulose derivatives from Scheme 1 are not regioselectively substituted. Even further, particular positions of substitution are shown in the scheme only for convenience of depiction. The following abbreviations are applicable: p-TSA, p-toluenesulfonic acid; DMF, N,N-dimethylformamide; Et₃N, triethylamine; MEK, methyl ethyl ketone; DMI, 1,3-dimethyl-2-imidazolidinone; and THF, tetrahydrofuran. The following chemical names are also applicable: PhCH₂OH, benzyl alcohol; (COCl)₂, oxalyl chloride; CH₂Cl₂, dichloromethane; H₂, hydrogen gas; Pd(OH)₂/C, palladium hydroxide on carbon catalyst. General methods of synthesis are known in the art and conventional techniques can be used to synthesize the polymers. See, e.g., Kar, N.; Liu, H.; Edgar, K. J. Biomacromolecules 2011, 12, 1106-1115.

For example, the inventors have developed a one-pot process for the synthesis of cellulose adipate alkanoates, by reaction of preformed cellulose esters in MEK or DMI solution with freshly prepared adipic anhydride. See, e.g., Liu, H.; Kar, N.; Edgar, K. J., “Direct synthesis of cellulose adipate derivatives using adipic anhydride,” Cellulose 2012, 19, 1279-1293. Process factors contributing to the success of such methods include the use of redistilled adipic anhydride, since poly(adipic anhydride), a universal contaminant in crude or aged adipic anhydride, resulted in crosslinked cellulose adipates. Dilution of the added adipic anhydride with solvent and careful kinetic studies to identify, and avoid, the onset of gelation are other essential elements to synthesis of a soluble cellulose adipate alkanoate that is not crosslinked. The absence of crosslinks can be confirmed both by spectroscopic analysis and product solubility. In fact, cellulose adipate alkanoates synthesized using this procedure tend to exhibit slightly enhanced organic solubility compared with their precursor cellulose esters. They have glass transition temperatures that, while reduced 30-50° C. in comparison with those of their precursor cellulose esters, nonetheless such derivative polymers typically exceed 100° C.

More specifically, synthesis methods for polymers of the invention can comprise one or more or all of the following process steps.

Reaction of Cellulose in DMAc/LiCl Solution with Commercial Adipic Anhydride.

MCC (8 g, 49.3 mmol) was completely dissolved in DMAc (300 mL) and LiCl (15 g) by a literature procedure (Edgar K J, Arnold K M, Blount W W, Lawniczak J E, Lowman D W (1995) Synthesis and properties of cellulose acetoacetates. Macromolecules 28:4122-4128). A pre-mixed solution of commercial adipic anhydride (6.31 g, 49.3 mmol) in DMAC (20 mL) was added dropwise to this solution at 80° C. under nitrogen. After approximately 45 min the solution gelled. The product was isolated by adding the reaction mixture to methanol, filtration of the gel-like material, and then extensive washing of the gel with methanol, then with water. The vacuum-dried product was insoluble in all solvents tried, including DMSO and chloroform. Product analysis was by infrared spectroscopy and solid state 13CNMR.

Preparation of Adipic Anhydride.

Adipic anhydride was synthesized by adapting a previously reported procedure. See Albertsson A C, Lundmark S (1990), Melt polymerization of adipic anhydride (Oxepane-2,7-Dione), J Macromol Sci Chem 27:397-412; and Albertsson A C, Eklund M (1996), Short methylene segment crosslinks in degradable aliphatic polyanhydride: network formation, characterization, and degradation, J Polym Sci Pol Chem 34:1395-1405. Adipic acid (15 g, 0.1026 mol) was dissolved in acetic anhydride (150 mL) in a three-neck round bottom flask. The reaction vessel was heated under reflux for 4 h with a continuous nitrogen purge. The by-product acetic acid and the excess acetic anhydride were removed by short path distillation under vacuum. The residue containing a small amount of acetic anhydride was transferred to a Claisen flask, and the depolymerization catalyst zinc acetate dehydrate (150 mg, 0.68 mmol) was added. The temperature was slowly raised under vacuum (1 mbar). The pure adipic anhydride fraction was collected at 80-95° C., and condensed in a flask cooled with an ice bath.

Procedure for the Reaction of CAP with Adipic Anhydride.

CAP-504-0.2 (1 g, 3.56 mmol) was dissolved in dry solvent (10 mL, MEK or DMI) and the solution was heated to 60 or 90° C. with stirring under nitrogen. Freshly synthesized adipic anhydride (0.396 g, 1 eq. per free hydroxyl group) was first dissolved in additional dry solvent (3 mL, MEK or DMI) and then added dropwise to the CAP solution. The resulting solution was stirred at 60 or 90° C. At timed intervals, aliquots of the reaction solution were withdrawn and added dropwise to isopropyl alcohol at room temperature with vigorous stirring. Each precipitate was collected by filtration. Each precipitate was further purified by re-dissolving in acetone, reprecipitating into water, then this precipitate was twice reslurried in hot water (90° C.) for 1 h each time, and each time recovered by filtration in order to remove residual adipic acid and poly(adipic anhydride). The final product was isolated by filtration, and vacuum-dried at 40° C.

¹H NMR (DMSO-d₆, ppm): 0.70-1.18 (COCH₂CH₃ of propionate), 1.35-1.65 (broad s, COCH₂CH₂CH₂CH₂CO of adipate), 1.85-2.50 (COCH₂CH₃ of propionate, COCH₃ of acetate and COCH₂CH₂CH₂CH₂CO of adipate), 3.20-5.30 (cellulose backbone).

¹³C NMR (DMSO-d₆, ppm): 173.8 (C═O of adipate), 172.0-173.2 (C═O of propionate), 101.9, 99.1 (C-1), 75.7 (C-4), 71.6-73.1 (C-2, C-3, C-5), 62.5 (C-6), 33.3 (COCH₂CH₂CH₂CH₂CO of adipate), 26.7 (COCH₂CH₃ of propionate), 23.8 (COCH₂CH₂CH₂CH₂CO of adipate), 8.6 (COCH₂CH₃ of propionate).

FTIR (KBr pellet method): 3,482 cm⁻¹, O—H stretching; 2,850-3,000 cm⁻¹, aliphatic C—H stretching; 1,738 cm⁻¹, ester and carboxylic acid C═O stretching.

A similar procedure was followed for the reactions of CAB-553-0.4 (1 g, 3.26 mmol) with freshly prepared adipic anhydride (0.363 g, 1 eq. per free hydroxyl group).

CAAdB Analytical Data: ¹H NMR (DMSO-d₆, ppm): 0.57-0.94 (COCH₂CH₂CH₃ of butyrate), 1.27-1.66 (COCH₂CH₂CH₃ of butyrate, COCH₂CH₂CH₂CH₂CO of adipate), 1.85-2.50 (COCH₂CH₂CH₃ of butyrate, COCH₃ of acetate and COCH₂CH₂CH₂CH₂CO of adipate), 3.20-5.30 (cellulose backbone).

¹³C NMR (DMSO-d₆, ppm): 173.8 (C═O of adipate), 172.0-173.2 (C═O of butyrate and acetate), 102.2, 99.5 (C-1), 75.5 (C-4), 71.3-72.3 (C-2, C-3, C-5), 62.4 (C-6), 35.2 (COCH₂CH₂CH₃ of butyrate), 33.3 (COCH₂CH₂CH₂CH₂CO of adipate), 23.9 (COCH₂CH₂CH₂CH₂CO of adipate), 20.3 (COCH₃ of acetate), 17.5 (COCH₂CH₂CH₃ of butyrate), 13.2 (COCH₂CH₂CH₃ of butyrate).

FTIR (KBr pellet method): 3,460 cm⁻¹, O—H stretching; 2,830-3,020 cm⁻¹, aliphatic C—H stretching; 1,741 cm⁻¹, ester and carboxylic acid C═O stretching.

Procedure for the Reaction of CA-398-30 with Adipic Anhydride.

CA-398-30 (1 g, 3.76 mmol) was dissolved in DMI (20 mL) and the solution was heated to 90° C. with stirring under nitrogen. Then freshly prepared adipic anhydride (0.766 g, 3 eq. per free hydroxyl group) was pre-dissolved in dry DMI (3 mL) and added dropwise. The solution was stirred at 90° C. for 20 h. The reaction mixture was then added to water at room temperature with stirring. The precipitate was collected by filtration and washed with hot water (90° C.). The product was further purified by Soxhlet extraction with isopropyl alcohol for 5 h, isolated by filtration, and finally vacuum-dried overnight at 40° C.

¹H NMR (DMSO-d₆, ppm): 1.34-1.59 (COCH₂CH₂CH₂CH₂CO of adipate), 1.68-2.13 (COCH₃ of acetate), 2.13-2.24 (COCH₂CH₂CH₂CH₂CO of adipate), 3.20-5.20 (cellulose backbone).

¹³C NMR (DMSO-d₆, ppm): 173.8 (C═O of adipate), 172.0-173.2 (C═O of acetate), 102.2, 99.5 (C-1), 75.5 (C-4), 71.3-72.3 (C-2, C-3, C-5), 62.4 (C-6), 33.3 (COCH₂CH₂CH₂CH₂CO of adipate), 23.9 (COCH₂CH₂CH₂CH₂CO of adipate), 20.3 (COCH₃ of acetate).

FTIR (KBr pellet method): 3,464 cm⁻¹, O—H stretching; 2,800-3,040 cm⁻¹, aliphatic C—H stretching; 1,740 cm⁻¹, ester and carboxylic acid C═O stretching.

A similar procedure was followed for the reaction of CA-320S (1 g, 4.19 mmol) with freshly prepared adipic anhydride (0.633 g, 1 eq. per free hydroxyl group). The reaction medium gelled within 2 h, and the product was not analyzed.

Indeed, any cellulose ester of the Formula I can be prepared by the same or similar methods to that provided in Scheme 1 and the methods described above. More specifically, virtually any cellulose adipate alkanoate can be made by the methods just above, especially the adipic anhydride procedure. Suberates and sebacates, however, do not form anhydrides and so for these polymers the monobenzyl ester monoacid chloride method with reagent prep as described in Scheme 1 can be used. Esters of Formula I include:

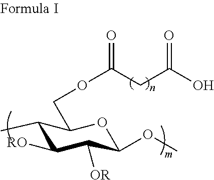

wherein n of the ω-carboxyalkanoyl group,

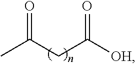

is 1, 2, 3, 4, 5, 6, 7, 8, 9, or 10;

wherein R is chosen from: a hydrogen atom; and an alkanoyl group; and

wherein m repeating units of the polymer ranges from 1 to 1,000,000, such as from 10 to 100,000, or from 100 to 1,000.

Note that the ester groups of the inventive polymers are preferably randomly distributed. In embodiments, the cellulose derivatives are not regioselectively substituted. Particular positions of substitution are shown in Scheme 1 only for convenience of depiction.

Solubility Studies.

Supersaturation is expressed as:

$$S = \frac{c}{{c}^{*}} \qquad ( Equation 11 )$$

where c is the concentration of the supersaturated solution and c* is the equilibrium solubility at a given temperature. The magnitude of S will be a major factor in determining how long supersaturation can be maintained in the absence and presence of seed crystals for nucleation induction time and crystal growth rate experiments, respectively. The equilibrium solubility of ritonavir at pH 6.8 and 37° C. is 1.3±0.1 μg/mL. The presence of additives in solution can change the equilibrium solubility of a drug compound; therefore it was important to investigate the impact of selected polymers on the equilibrium solubility of crystalline ritonavir at the desired and effective polymer concentration of 5 μg/mL. The equilibrium solution concentration of ritonavir in the presence of selected polymers at a concentration of 5 μg/mL is summarized in Table 4. The effect of polymers on the equilibrium solubility of crystalline ritonavir at a polymer concentration of 5 μg/mL is negligible.

TABLE 4
Equilibrium solubility of ritonavir in the presence of polymer at a
concentration of 5 μg/mL

| Polymer | Solubility^{a} of Ritonavir (μg/mL) |
|---|---|
| PVP | 1.2 ± 0.05 |
| PVPVA | 1.3 ± 0.03 |
| HPMC | 1.5 ± 0.02 |
| HPMCAS | 1.4 ± 0.09 |
| CAP 504-0.2 Adp | 1.6 ± 0.10 |
| CAP Adp 3X | 1.3 ± 0.02 |
| CAB Adp 3X | 1.3 ± 0.01 |
| CMCAB | 1.6 ± 0.01 |
| Pn-IPAAmd | 1.3 ± 0.01 |

^{a}The solubility of ritonavir at pH 6.8 and 37° C. in the absence of polymer is 1.3 ± 0.10 μg/mL.

Dissolution of Amorphous Ritonavir.

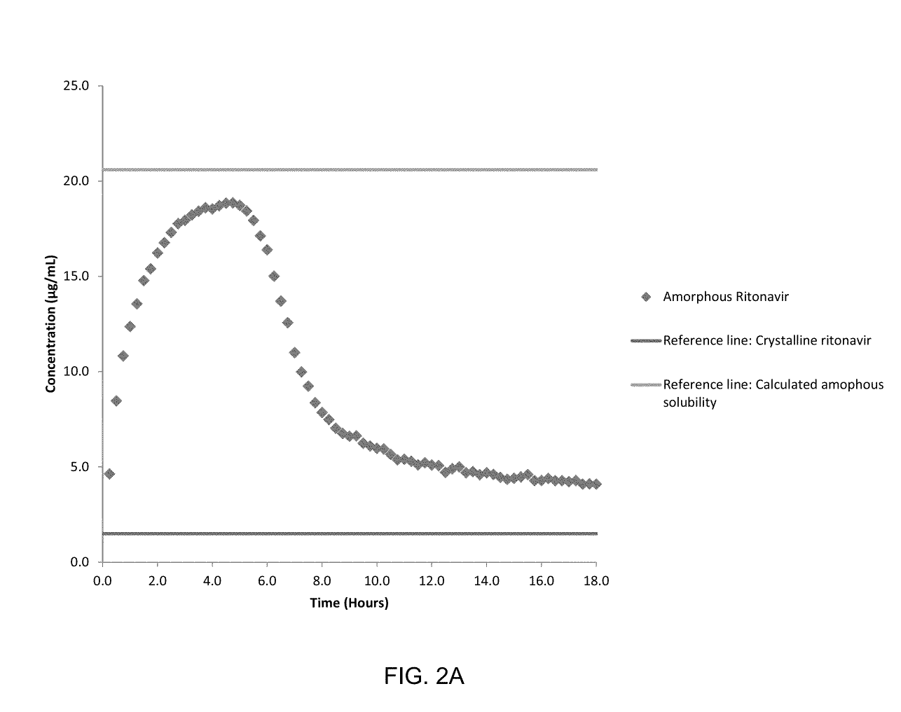

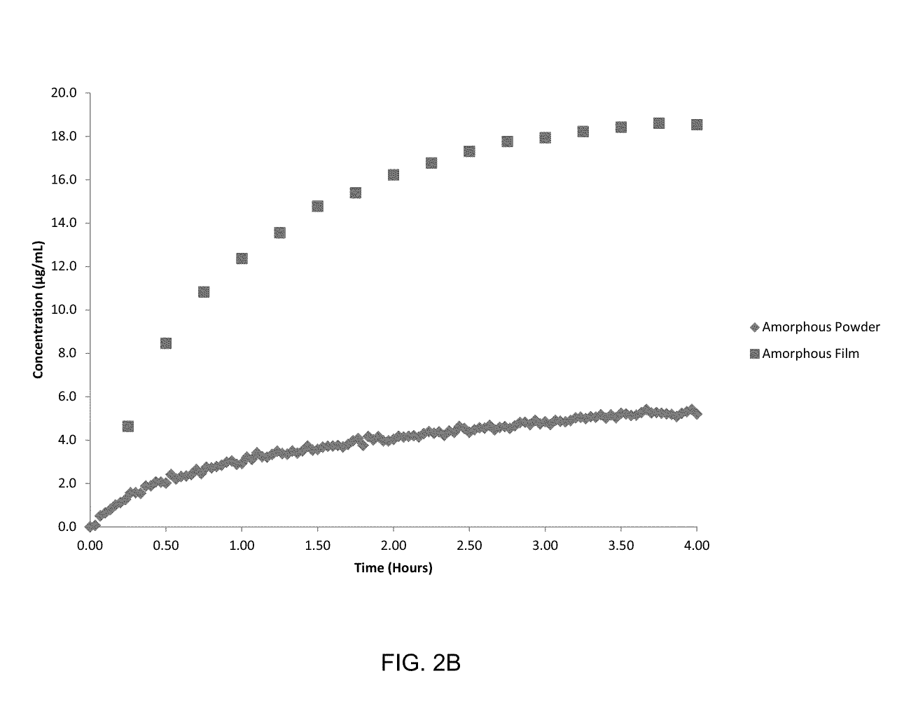

FIG. 2A is a graph showing dissolution of amorphous ritonavir using an amorphous film. The red and green lines are the crystalline and calculated amorphous solubility references, respectively. FIG. 2B is a graph showing dissolution of the powder form of amorphous ritonavir prepared by the melt-quench method. The powder agglomerates in solution, thereby reducing the surface area available for dissolution. Consequently, the dissolution rate of powdered amorphous ritonavir is low.

FIG. 2A shows the dissolution profile for amorphous ritonavir at 37° C. The crystalline solubility line is included for reference. It should be noted that all dissolution experiments were performed under non-sink conditions in order to evaluate the maximum solution concentration generated from an amorphous solid. So, the amount of solid added was in excess of the amount required to reach the equilibrium solubility of ritonavir. The maximum solution concentration generated by the amorphous ritonavir was 18.8 μg/mL, followed by rapid desupersaturation. The experimental maximum solution concentration is very close to the calculated amorphous solubility of ritonavir with moisture sorption correction (20.6 μg/mL). From these results, it can be concluded that the level of supersaturation attained during dissolution of amorphous ritonavir depends on the crystallization tendency of the generated supersaturated solution. The advantage of using an amorphous film for the dissolution of ritonavir is illustrated in FIG. 2B. A constant surface area of the amorphous film resulted in an increase in dissolution rate for ritonavir in comparison to dissolution of powder form of ritonavir prepared by melt quenching. With the use of this new method, the maximum solution concentration for using amorphous RTV was attained in approximately 4 hours.

Nucleation—Induction Time.

The experimental induction time of precipitation can be described as the time which elapses between the creation of supersaturation and the first detectable change in some physical property of the precipitating system, e.g. appearance of crystals or turbidity. See Sohnel 1982. Therefore, the experimental induction time is dependent on the sensitivity of the method used.

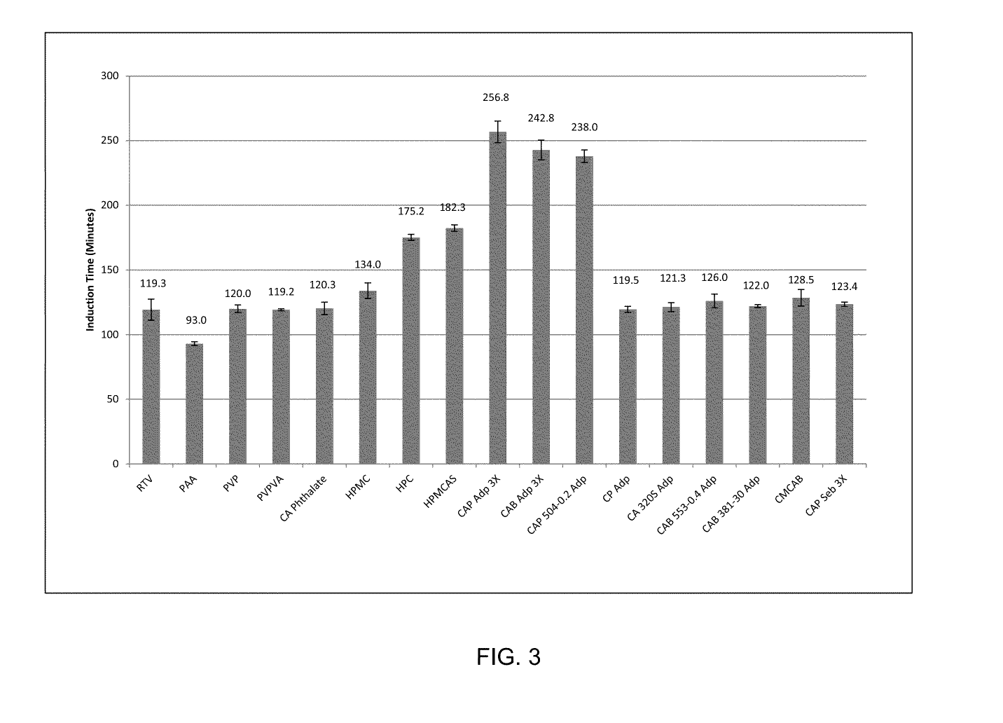

The method used in the context of this specification is sensitive and gives more accurate values compared to visual inspection. Visual inspection, however, can alternatively be used or additionally used for confirmation. The induction times of ritonavir at a supersaturation of 20 in the absence and presence of a number of polymers are presented in FIG. 3. The nucleation-induction time of ritonavir at an initial concentration of 20 μg/mL, in the absence and presence of polymers at a concentration of 5 μg/mL, is provided in FIG. 3.

Ritonavir, which crystallizes slowly in solution, has an induction time of approximately 119 minutes. Most of the commercially available polymers that were evaluated (e.g. PVP, CMCAB and HPMC), were unable to inhibit nucleation. However, the novel polymers CAP Adp 3X, CAB Adp 3X and CAP 504-0.2 Adp significantly prolonged the induction time, with CAP Adp 3X being the most effective of the polymers.

Nucleation involves the diffusion of molecules through the bulk of the solution, collision with each other, and formation of nuclei of a critical size. See Mullin, J. W., Crystallization, 4th edition, Oxford: Elsiever Butterworth-Heinemann (2001). When the critical size is reached, crystal growth can subsequently occur by the diffusion of molecules to the crystal surface and the incorporation of the molecules in the solid phase. See Matteucci, M. E., Hotze, M. A., Johnston, K. P., and Williams, R. O., “Drug nanoparticles by antisolvent precipitation: mixing energy versus surfactant stabilization,” Langmuir 22: 8951-8959 (2006).

Nucleation can be inhibited by hindering the aggregation of crystals by steric or electrostatic stabilization through the adsorption of polymeric additives to the surface of the newly formed crystals. See Zimmermann, A., Millqvist-Fureby, A., Elema, M. R., Hansen, T., Mullertz, A. and Hovgaard, L., “Adsorption of pharmaceutical excipients onto microcrystals of siramesine hydrochloride: Effects of physicochemical properties,” European Journal of Pharmaceutics and Biopharmaceutics, 71: 109-116 (2009).

The presence of a polymeric additive, even at very low concentrations, can have a great effect on nucleation and crystal growth rate as well as crystal morphology. Although it is virtually impossible to generate a general mechanism to explain the effects of additives on nucleation, crystal growth and crystal morphology (see Mullin 2001), there is a general consensus that the adsorption of additive molecules on the surface of crystal is a required step for the stabilization to occur. Some of the mechanisms that have been proposed in the literature for inhibition of nucleation by a polymeric additive include: presence and strength of specific interactions between drug and polymer, such as hydrogen bonding. See Raghavan, S. L., Trividic, A., Davis, A. F. and Hadgraft, J., “Crystallization of hydrocortisone acetate: influence of polymers,” International Journal of Pharmaceuticals, 212: 213-221 (2001). However, in aqueous solution, strong interactions between polymers and drugs are important, but may not be sufficient to inhibit nucleation from the supersaturated solutions. See Ziller, K. H. and Rupprecht, H. H., “Control of crystal growth in drug suspensions 2: Influence of polymers on dissolution and crystallization during temperature cycling,” Drug Development and Industrial Pharmacy, 52: 1017-1022 (1990). The key nucleation inhibitory mechanism by these polymers, however, is not fully understood and is currently under investigation.

Crystal Growth Rate.

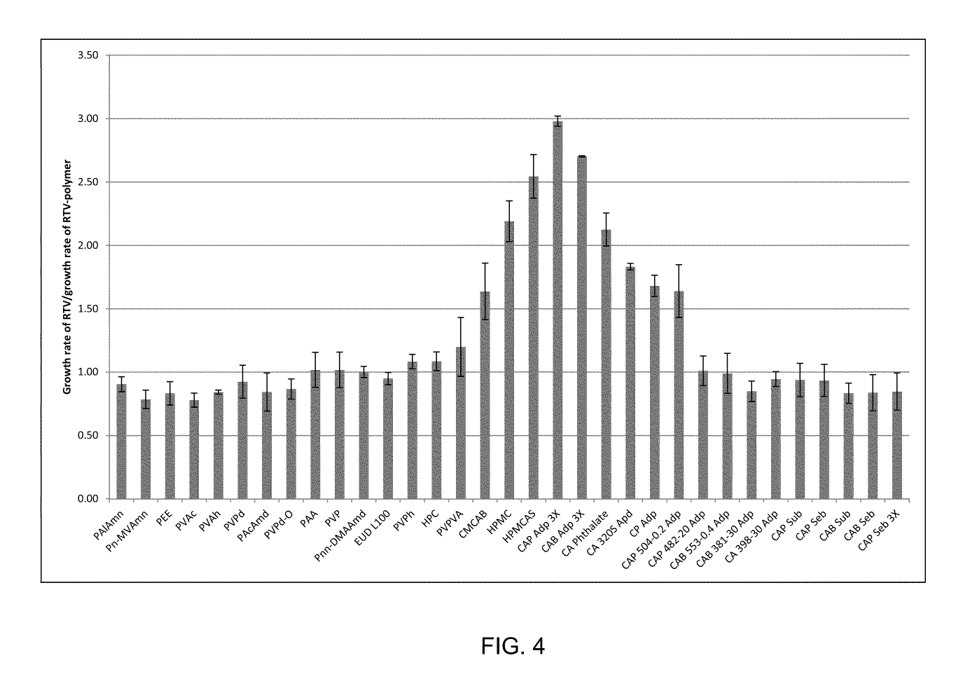

A bar graph comparing the growth rate of ritonavir (S=10) in the absence of polymer to the growth rate of ritonavir in the presence of polymer (5 μg/mL) is shown in FIG. 4. More specifically, FIG. 4 shows the crystal growth rate of ritonavir in the presence of polymers, where the y-axis is a ratio of the growth rate of ritonavir in the absence of polymer to growth rate of ritonavir in the presence of polymer. Polymers with a ratio>1 are considered effective crystal growth inhibitors.

Also included within the scope of the present invention is a composition for use in the treatment of a disease chosen from at least one of AIDS, HIV, or cancer by administering an effective amount of the composition to a subject with the disease, wherein the composition comprises: at least one amorphous drug with a solubility of less than about 1 mg/mL; at least one first polymer chosen from cellulose esters of formula I:

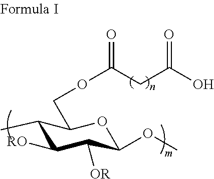

wherein n of the ω-carboxyalkanoyl group

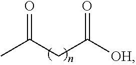

is 1, 2, 3, 4, 5, 6, 7, 8, 9, or 10; wherein R is chosen from: a hydrogen atom; and an alkanoyl group. With respect to the number of repeating units of the polymer, m, can range from 1 to 1,000,000, such as from 1 to 500,000, or from 1 to 100,000, or from 1 to 50,000, or from 1 to 10,000. Preferred polymers have n number of repeating units ranging from 1 to 1,000,000, such as from 10 to 100,000, or from 100 to 1,000. Such compositions can comprise a polymer with a total degree of substitution of the alkanoyl group and the ω-carboxyalkanoyl group of at least 2.0. In preferred embodiments, the compositions can comprise a polymer wherein the alkanoyl group is chosen from at least one of acetyl, propionyl, butyryl, valeroyl, hexanoyl, nonanoyl, decanoyl, lauroyl, palmitoyl, and stearoyl groups. The compositions can comprise a polymer wherein the ω-carboxyalkanoyl group is chosen from at least one of succinoyl, glutaroyl, adipoyl, sebacyl, and suberyl groups.

A wide range of polymers with different chemical structures and containing different functional groups were considered. Some of the commercially available polymers were effective in inhibiting crystal growth from solution, however the majority of the polymers were ineffective. Out of the 19 commercially available polymers investigated, only four were effective (HPMC, HPMCAS, CA Phthalate and CMCAB). Even then, the novel synthesized cellulose derivatives, CAP Adp 3X and CAB Adp 3X were more effective in inhibiting crystal growth compared to the aforementioned commercially available polymers. Five out of the 14 novel synthesized cellulose derivatives were effective crystal growth inhibitors to varying extents. Further, CASub and CASeb were even more effective. For example, CASub with DS (Ac) of about 1.8 and DS (Sub) of about 0.9 is absolutely outstandingly effective.

Polymer adsorption affects solution crystal growth by blocking the sites for incorporation of new growth units. Thus, crystal growth is inhibited and consequently crystals of reduced size are formed. See Zimmermann 2009. In general, the more hydrophilic polymers, commercially available polymers, which are represented on the left-hand side of FIG. 4, are ineffective crystal growth inhibitors. Also, the more hydrophobic novel polymers, which are represented on the right-hand side of the figure, are ineffective. From this result, it appears that hydrophobicity is an important factor in crystal growth inhibition. Hydrophobicity of the polymer may affect the extent and nature of adsorption of polymer to the crystal surface and in turn may influence the effectiveness of the polymer as a crystal growth inhibitor. If the polymer is too hydrophilic, it may interact more favorably with the solvent molecules and if the polymer is too hydrophobic, it may interact more favorably with other like polymer molecules in solution, instead of adsorbing to the drug surface. Strong polymer-polymer interaction in the solution and at the interface can lead to coadsorption of one polymer which otherwise does not adsorb to the crystal surface. See Yu, X. and Somasundaran, P., “Role of polymer conformation in interparticle-bridging dominated flocculation,” Journal of Colloid and Interface Science, 177: 283-287 (1996). Consequently, there appears to be an optimal level of hydrophobicity a polymer should have for it to be an effective crystal growth inhibitor; that is, there has to be hydrophilic/hydrophobic balance, while enough of the polymer must dissolve in aqueous media to be effective.

The importance of hydrophobicity in adsorption was also highlighted in the study conducted by Zimmermann et al (2009). The authors found out that polymers that contain hydrophobic substituents (HPMC and HPC) have a higher affinity for hydrophobic drug particles, whereas more a more hydrophilic polymer (PEG) did not adsorb to the hydrophobic drug particle surface. See Zimmermann 2009. In addition, Tian et al. studied the influence of various excipients on growth of carbamazepine dihydrate crystals from anhydrous carbamazepine. The solubility parameter was used to describe the hydrophobicity of the excipients. HEC, which had the highest solubility parameter, did not inhibit crystal growth to the same extent as HPMC and HPC, which had lower solubility parameters. Of all tested polymers, HPMC most effectively inhibited growth of dihydrate crystals. See. Tian, F., Saville, D. J., Gordon, K. C., Strachan, C. J., Zeitler, J. A., Sander, N. Rades, T., “The influence of various excipients on the conversion kinetics of carbamezepine polymers in aqueous suspension,” Journal of Pharmacy and Pharmacology, 59: 193-201 (2007).

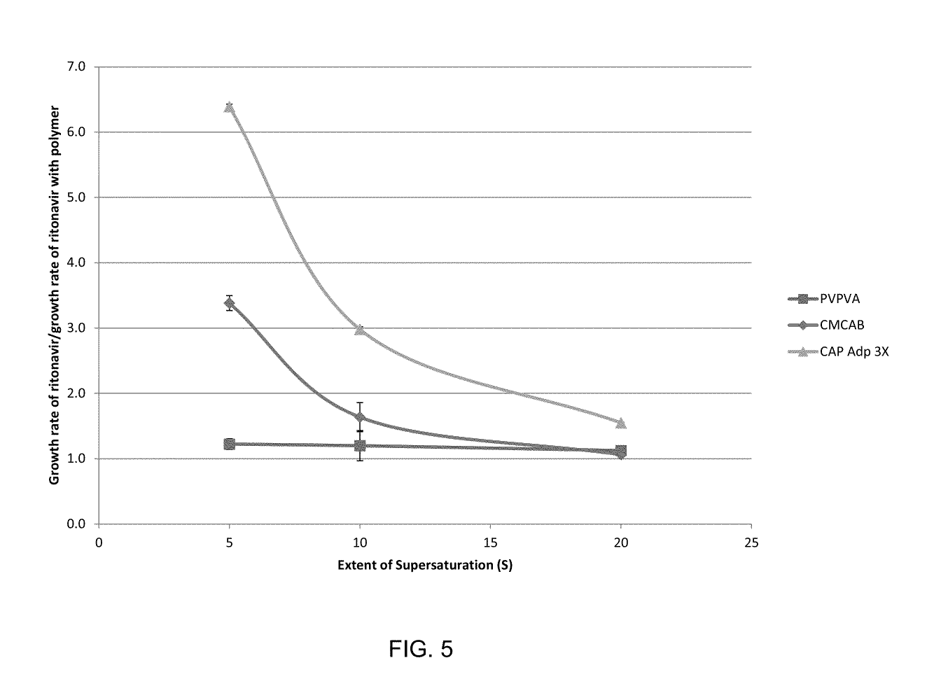

The impact of supersaturation on crystal growth rate was investigated and the result is depicted in FIG. 5. Shown in FIG. 5 is the crystal growth rate of ritonavir as a function of ritonavir supersaturation. The y-axis is a ratio of the growth rate of ritonavir in the absence of polymer to the growth rate of ritonavir in the presence of polymer at each supersaturation.

PVPVA was ineffective in inhibiting crystal growth at all supersaturations, while CMCAB was ineffective only at a supersaturation of 20, which corresponds to the maximum level of supersaturation observed during the dissolution of amorphous ritonavir. The novel polymer CAP Adp 3X was more effective at lower supersaturations of 5 and 10; it was slightly effective at a supersaturation of 20. As would be expected from classical crystallization theory, the rate of crystal growth increased as a function of supersaturation in the presence and absence of the polymers. It is important to recall that nucleation-induction time experiments were performed at a supersaturation of 20 and a polymer concentration of 5 μg/mL, similar to the crystal growth rate experiments. With respect to nucleation, it was found that at a polymer concentration of 5 μg/mL, CAP Adp 3X substantially inhibited nucleation. As provided in more detail below, CA 320 S suberate can inhibit crystal growth to some extent at for example 20 μg/mL. It can be concluded that inhibiting nucleation may be more important than inhibiting growth when considering the stability of a supersaturated solution.

Hydrophobicity of Cellulose Derivatives.

It would appear there are some key structure-property relationships of the novel synthesized polymers that enable the inhibition of crystal growth. One such factor may be the importance of hydrophobicity within the novel synthesized cellulose derivatives. The solubility parameters of the novel polymers were used to characterize their relative hydrophobicity. This information is contained in Table 2. The solubility parameter (δ) provides a numerical estimate of the degree of interaction between materials, and can be a good indication of solubility, particularly for non-polar materials. The solubility parameter of water is 49.01 MPa^{1/2}. Generally, the higher the solubility parameter of a polymer, the more hydrophilic it is. CP Adp, one of the more hydrophilic of the novel polymers, has a δ of 23.28 MPa^{1/2}, while CAB Seb, one of the more hydrophobic of the novel polymers, has a δ of 19.62 MPa^{1/2}. The hydrophobicity ranking of these polymers is presented in Table 2, where the least hydrophobic polymer, CP Adp is ranked #1 and #14 represents the most hydrophobic polymer, CAB Seb.

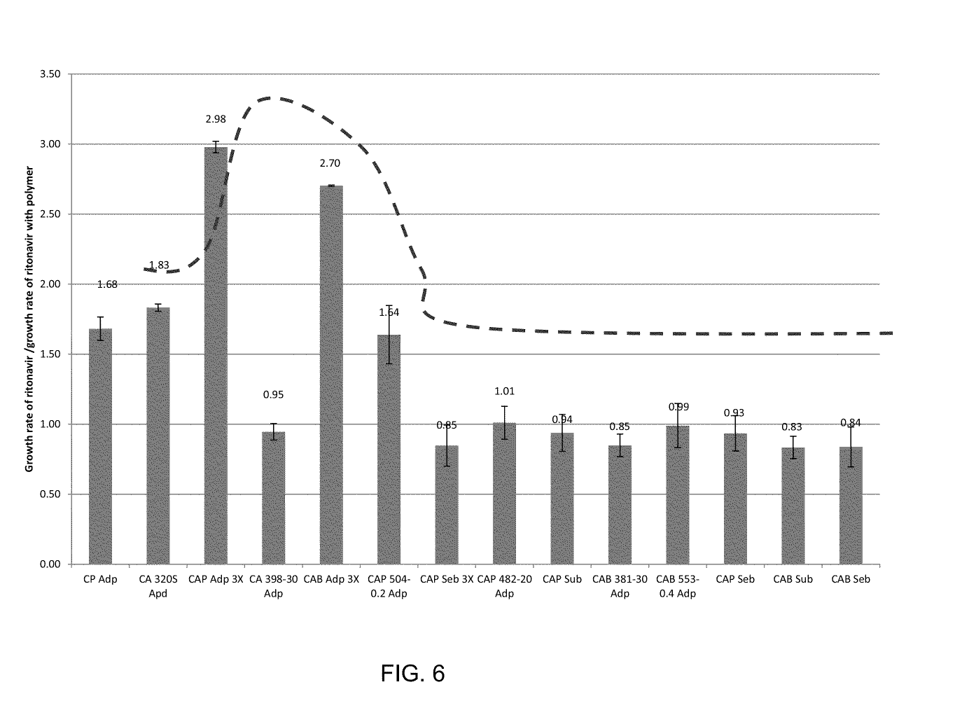

The crystal growth rate results for the novel polymers arranged in order of hydrophobicity (least to most hydrophobic, L to R) are presented in FIG. 6. More specifically, FIG. 6 shows the crystal growth rate of ritonavir at an initial solution concentration of 10 μg/mL in the presence of novel synthesized cellulose derivatives (5 μg/mL). The data are arranged in order of hydrophobicity: least hydrophobic to most hydrophobic (left to right). The trend in growth rate is highlighted using the red-dashed line. Clearly, there is a trend, with the exception of CA 398-30 Adp. The polymers with a δ between 20.56-23.28 MPa^{1/2} inhibited crystal growth, while polymers with δ<23.28 MPa^{1/2} were ineffective. The polymers with the ideal level of hydrophobicity, CAP Adp 3X and CAB Adp 3X, were the most effective crystal growth inhibitors. This trend reflects the importance of polymer hydrophobicity as a key factor that influences crystallization inhibition by a polymer.

Importance of Ionizable Groups.

The degree of substitution (DS) of a given cellulose polymer is defined as the average number of substituted hydroxyl groups per cellulose chain. The rank of these polymers based on DS of the adipate substituent is presented in Table 2, where the polymer with the highest DS (Adp), CAP Adp 3X is ranked #1 and #11 represents the polymer(s) with the lowest DS (Adp), CAB 381-30 Adp and CAP 482-20 Adp. The pKa of the adipate substituent group is approximately 4.43, so the degree of ionization of the adipate substitution group will be nearly complete (^{˜}100%) at pH 6.8.

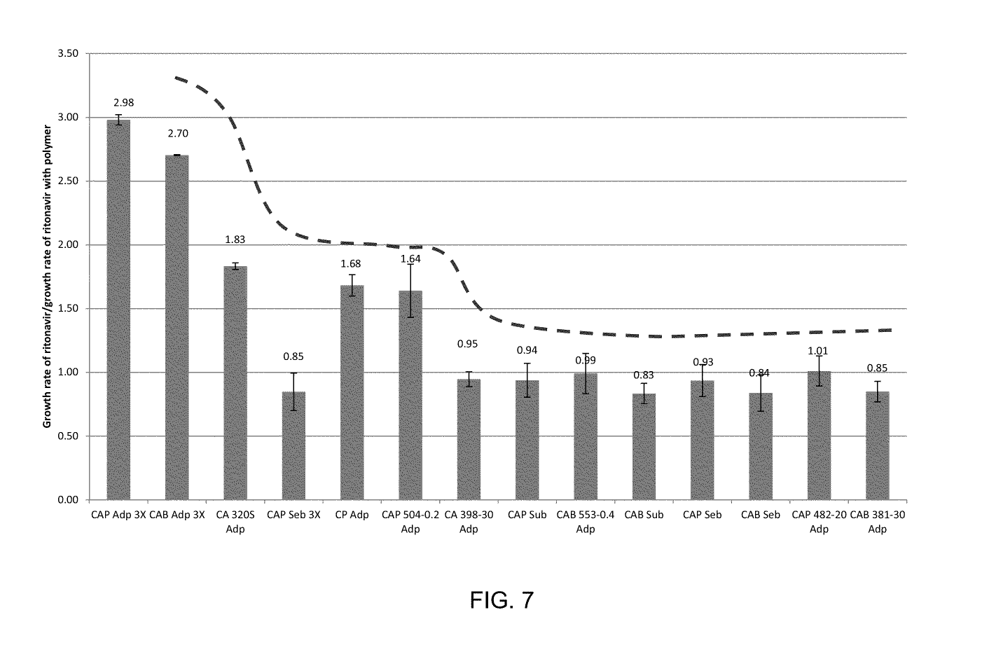

The crystal growth rate result for the novel polymers, arranged in order of decreasing DS (Adp) (highest to lowest DS (Adp)), is presented in FIG. 7. More specifically, FIG. 7 shows crystal growth rate of ritonavir at an initial solution concentration of 10 μg/mL in the presence of novel cellulose derivatives (5 μg/mL). The polymers are arranged in order of degree of substitution of the adipate group: high to low DS (Adp) from left to right.

The trend in growth rate is highlighted using the red-dashed line. It would appear there is a trend, with the exception of CAP Seb 3X. The ability of the polymers to inhibit crystal growth decreases with decreasing DS (Adp), that is, the higher the DS, the higher the number of anionic groups in solution at pH 6.8. This way crystal growth can be inhibited by electrostatic stabilization of the adsorbed polymer. Moreover, the presence of ionic groups on the polymer surface may alter the conformation of the polymer. A polymer with extended end groups may cover the surface of the crystal more effectively.

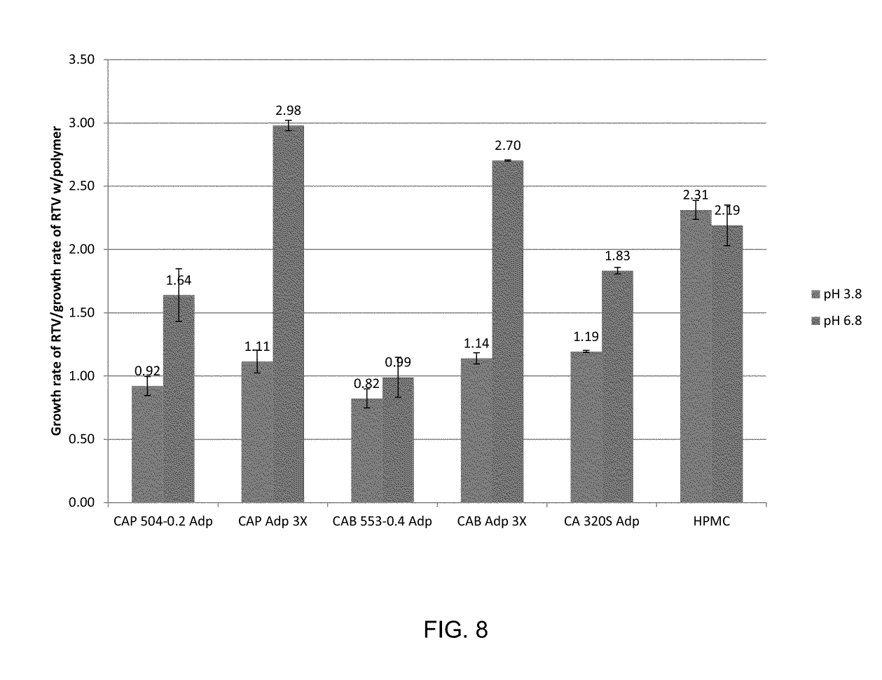

The influence of ionizable groups on crystal growth was further investigated by performing growth rate experiments at pH 3.8. At pH 3.8 the adipate substituent is only 23% ionized. FIG. 8 compares crystal growth rate inhibition for selected polymers at pH 3.8 and 6.8. In FIG. 8, the crystal growth rate of ritonavir at two pH conditions, 3.8 and 6.8, is shown, with initial ritonavir solution concentration of 10 μg/mL and polymer concentration of 5 μg/mL.

The effectiveness of HPMC, a non-ionizable polymer, did not decrease at pH 3.8. On the other hand, the most significant decrease in polymer effectiveness at pH 3.8 (63% reduction) was observed for CAP Adp 3X, which has the highest number of ionizable groups (DS (Adp)=0.85), while the smallest decrease in effectiveness (17% reduction) was observed for CAB 553-0.4 Adp. Of all the polymers evaluated at pH 3.8, CAB 553-0.4 Adp has the lowest DS (Adp) (0.25) and it is also the most hydrophobic.

Acrylic acid, the repeating unit of PAA has a pKa of 4.76. See Laguecir, A., Ulrich, S., Labille, J., Fatin-Rouge, N., Stoll, S. Buffle, J., “Size and pH effect on electrical and conformational behavior of poly(acrylic acid): Simulation and experiment,” European Polymer Journal, 42: 1135-1144 (2006). A number of studies have investigated the impact of pH on the conformation of polyacrylic acid (PAA). See Laguecir 2006; Yu, X. and Somasundaran, P., “Role of polymer conformation in interparticle-bridging dominated flocculation,” Journal of Colloid and Interface Science, 177: 283-287 (1996); and Tjipangandjara, K. F. and Somasundaran, P., “Effects of changes in adsorbed polyacrylic acid conformation on alumina flocculation. Colloids and Surfaces,” 55: 245-255 (1991). Both the novel cellulose synthesized derivatives and PAA, with carboxyl groups, can be expected to possess different conformations at different pH value's due to the pH dependent ionization ratio of COOH groups and interactions of these groups with other ions in the system. See Yu 1996. Yu et al. (1996) used a fluorescence spectroscopic technique to determine the conformation of pyrene labeled PAA in alumina supernatant. The authors observed that the polymer chain becomes stretched out with increase in pH due to the increasing intramolecular electrostatic repulsion caused by the ionization of COOH groups.

Tjipangandjara et al. (1991) reported similar findings, but in addition, the authors investigated the effect of PAA concentration on the conformation of adsorbed species. At low polymer concentrations (5 μg/mL), increase in pH caused coiled polyacrylic acid on the surface to stretch out (low surface coverage), whereas stretching is less at the higher polymer concentration (100 μg/mL) due to crowding of the polymer chains on the particles because of larger surface coverage and ensuing restricted expansion of the adsorbed coils. The change in conformation of these polymers with pH and concentration suggests that effectiveness of a polymer may not only be directly related to the extent of adsorption, but also on the extent of surface coverage and the interactions among the adsorbed polymer molecules. Even though the authors observed low surface coverage of PAA at 5 μg/mL (a concentration used in examples herein), this may not be the case for the novel synthesized cellulose derivatives.

In addition, Laguecir et al. (2006) investigated the conformational behavior of charged PAA by calculating the mean square radius of gyration [symbol] [symbol] [symbol] for three different grades of PAA with different molecular weights. The authors reported a global increase in [symbol] [symbol] [symbol] with pH and ionization degree. When pH is high, PAA is almost fully charged and long range interactions are strong enough to extend conformations and increase [symbol] [symbol] [symbol] . Therefore, from the discussion above it can be concluded that decreasing the pH of the test media, reduces the degree of ionization of the novel polymer which results in a change of polymer conformation. This change in polymer conformation is believed to influence the inhibitory capacity of the novel polymers of the invention. It is believed that the polymer coils up on itself, thus reducing surface coverage and the interactions among the adsorbed polymer molecules.

Outliers in FIGS. 6 and 7 are highlighted in yellow in Table 2. The results and discussion above have emphasized the importance of hydrophobicity and ionizable groups on the effectiveness of the novel synthesized cellulose derivatives. These two key factors are jointly responsible for the stabilizing ability of the novel polymers; they are interrelated and influence one another. Even though CA 398-30 Adp is characterized as a relatively less hydrophobic polymer (hydrophobicity rank #4), it has a low number of ionizable groups (DS (Adp)=0.21, rank #10) compared to the effective polymers, CAP Adp 3X and CAB Adp 3X (FIG. 6). CAB Seb 3X is an outlier in FIG. 7 because, although it has a high number of ionizable groups (DS (Adp)=0.67, rank #3), it is a relatively more hydrophobic polymer (hydrophobic rank #7). For these reasons, CA 398-30 Adp and CAB Seb 3X were outliers.

The effect of hydrophobicity on polymer adsorption alone does not adequately explain why Pn-IPAAmd, a synthetic and moderately hydrophobic polymer, is the most effective in inhibiting crystal growth of ritonavir. It is speculated that Pn-IPAAmd adsorbs to one or more of the fast growing faces of ritonavir crystals through the formation of specific intermolecular interactions. To gain a better insight into the impact of Pn-IPAAmd upon ritonavir crystallization, the crystals extracted after crystal growth in the absence and presence of the predissolved polymer were analyzed using SEM and PXRD. The aspect ratio of ritonavir crystals after growth in the absence of polymer increased (from 7.7 to 8.8, before and after growth, respectively) since the crystals grew length-wise and became more elongated along the a-axis. There are four faces shown by the BFDH morphology predictor (FIG. 4e) that grow approximately perpendicular to the a-axis and are the fastest growing faces [(−10−1), (−1−10), (−101), (−110)]. These planes expose functional groups containing electronegative atoms such as carbonyl oxygen and nitrogen atoms from the thiazole functional group of ritonavir, which (assuming these are also the fast growing faces in the experimental system) can potentially interact with the amide group of Pn-IPAAmd. In the presence of predissolved Pn-IPAAmd, the crystals appeared to be less elongated, suggesting adsorption of Pn-IPAAmd to the fast growing faces, resulting in an increase in width (from 5.7±1.4 to 9.3±2.5 μm, in the absence and presence of predissolved Pn-IPAAmd, respectively) and in turn, a reduced average aspect ratio of the crystals. Therefore, the strong crystal growth inhibitory ability of Pn-IPAAmd can likely be attributed to a combination of the moderate hydrophobicity of the polymer and fortuitous intermolecular hydrogen bonding to the fast-growing faces at the solid-liquid interface. The other amide polymers that were investigated (Pnn-DMAAmd and PAcAmd) were probably ineffective because they were either too hydrophilic or have no hydrogen bond donor groups as illustrated in Formula II below:

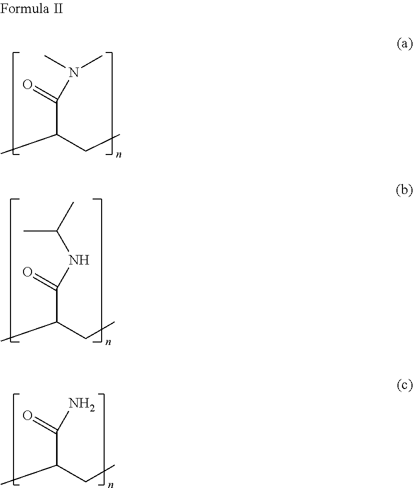

wherein n is an integer from 1 to 1,000,000, and wherein (a), (b), and (c) are the molecular structures of various synthetic amide polymers that can be used in embodiments of the invention, namely, (a) Pnn-DMAAmd (poly(N,N-dimethyl acrylamide)), (b) Pn-IPAAmd (poly(N-iso-propylacrylamide)), and (c) PAcAmd (poly(acrylamide)).

Dissolution of Amorphous Solid Dispersions.

It is has been shown that dissolution of an amorphous solid generates higher solution concentrations than that of the crystalline form (Section 4.2). However, ritonavir crystallizes from solution because of the strong driving force for crystallization due to supersaturation. Once a supersaturated solution is created and the solution concentration is in the labile zone, spontaneous nucleation followed by crystal growth will occur. This would result in a reduction of solution concentration towards the thermodynamic solubility. The rate of this desupersaturation must be reduced in order to have an increase in bioavailability.

In some circumstances, there is a relationship between low bioavailability of a drug and solubility of the drug. Solubility is often a critical issue in drug delivery in that the drug is not capable of permeating the epithelium and reaching the bloodstream if it is not first dissolved in the aqueous gastrointestinal lumen. According to the Biopharmaceutics Classification System (BCS), BCS Class II type drugs are characterized by having high intestinal permeability but low solubility. It is understood that enhancing solubility of a BCS Class II compound almost invariably gives higher bioavailability. As oral drug delivery is preferred by patients and it is highly desirable to convert other delivery modes to oral, where possible, it would be highly desirable to enhance solubility of certain drugs to increase their bioavailability. Accordingly, it is especially preferred to use the polymers and polymer combinations of the invention with any one or more BCS Class II or Class IV drug. Indeed, the compositions of the invention can additionally or alternatively incorporate BCS Class I and II drugs as well.

Specific drugs that can be used in compositions of the invention include, for example, any one or more of, Acetazolamide, Albendazole (antiparasitic), Allopurinol, Amitriptyline (antidepressant), Amlodipine, Artemether+Lumefantrine (antimalarial agents), Azathioprine, Besilate, Candesartan, Carbamazepine, Celecoxib, Chlorpromazine (antidepressant), Cilexetil, Ciprofloxacin (antibiotic), Clarithromycin, Clofazimine (antibacterial agent), Clotrimazole, Clofazimine, Colchicine, Curcurmin or Curcumin, Cyclosporin A, Danazol, Dapsone, Diazepam, Diloxanide (antiprotozoal agent), Diloxanide Furoate, Doxycycline, Efavirenz (antiviral), Ellagic Acid, Ethinyl Estradiol (hormone), Etravirine, Ezetimibe, Folic acid, Furosemide, Glibenclamide (antidiabetic), Griseofulvin, Haloperidol (neuroleptic), Ibuprofen, Itraconazole, Lopinavir (antiviral), Lopinavir (with Ritonavir), Lumefantrine (with Artemether), Mebendazole (antihelmintic), Mebendazole (chewable), Mefloquine (antimalarial), Nalidixic acid (antibacterial agent), Naringenin Nevirapine (antiviral), Niclosamide (anthelmintic), Niclosamide (chewable), Nifedipine, Nitrofurantoin, Paclitaxe, Paracetamol, Phenyloin (chewable), Praziquental (antihelmintic), Primaquine, Pyrantel (anthelmintic), Pyrantel Embonate, Pyrimethamine Toxoplasmose, Pyrimethamine, Quercetin, Retinol (vitamin), Retinol Palmitate, Resveratrol, Rifampicin (antituberculotic), Ritonavir, Saquinavir, Spironolactone (diuretic), Sulfadiazine (antibacterial agent), Sulfamethoxazole, Sulfasalazine, Sulfasalazine Colitis Ulcerosa/Morbus Crohn, Theophylline, Triclabendazole (anthelmentic), Trimethoprim, Verapamil Hydrochloride (Ca-channel blocker), Warfarin Sodium (anticoagulant), or any one or more of these drugs in combination with any other drug.

Examples of specific compositions according to embodiments of the invention include any one or more of the drugs listed in this specification in combination with any one or more polymer identified in this specification, wherein the drug(s) and polymer(s) are present in the composition in a drug:polymer ratio ranging from 0.01:99.09 to 99.09:0.01, such as from 0.05:99.05, 0.1:99.9, or 0.5:99.5, or 1:99, or 5:95, or 10:90, or 15:85, or 20:80, or 25:75, or 30:70, or 35:65, or 40:60, or 45:55, or 50:50, or 55:45, or 60:40, or 65:35, or 70:30, or 75:25, or 80:20, or 85:15, or 90:10, or 95:100, and so on. Virtually any drug and polymer combination and any drug:polymer ratio can be used in compositions of the invention.

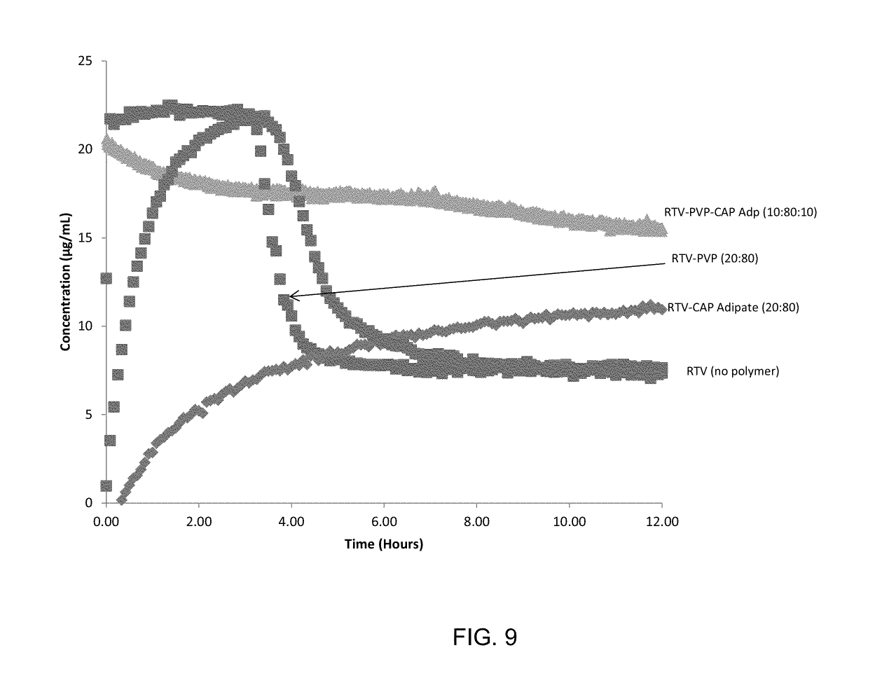

The dissolution behavior of amorphous solid dispersions containing ritonavir incorporated in different polymeric carriers was investigated at a ratio of 20% drug and 80% polymer. The polymers investigated were PVP and CAP 504-0.2 Adp. FIG. 9 shows the apparent concentration-time profiles of the amorphous solid dispersions. More specifically, FIG. 9 shows dissolution of amorphous solid dispersions of ritonavir using a binary combination of PVP and CAP 504-0.2 Adp, which is able to maintain solution concentration close to the amorphous solubility of ritonavir.

The solid dispersion containing 80% PVP resulted in a significant increase in dissolution rate. However, PVP was unable to extend the duration of supersaturation, compared to pure amorphous ritonavir, because PVP was ineffective at inhibiting both nucleation and crystal growth of ritonavir. When amorphous solid dispersions containing 80% CAP 504-0.2 Adp were dissolved, the maximum apparent solution concentration was only 10 μg/mL. Although the novel synthesized cellulose derivative is an effective crystallization inhibitor, an amorphous solid dispersion containing this hydrophobic polymer at an 80% polymer concentration had a slower dissolution rate compared to pure amorphous ritonavir (i.e. the dissolution rate was polymer controlled).

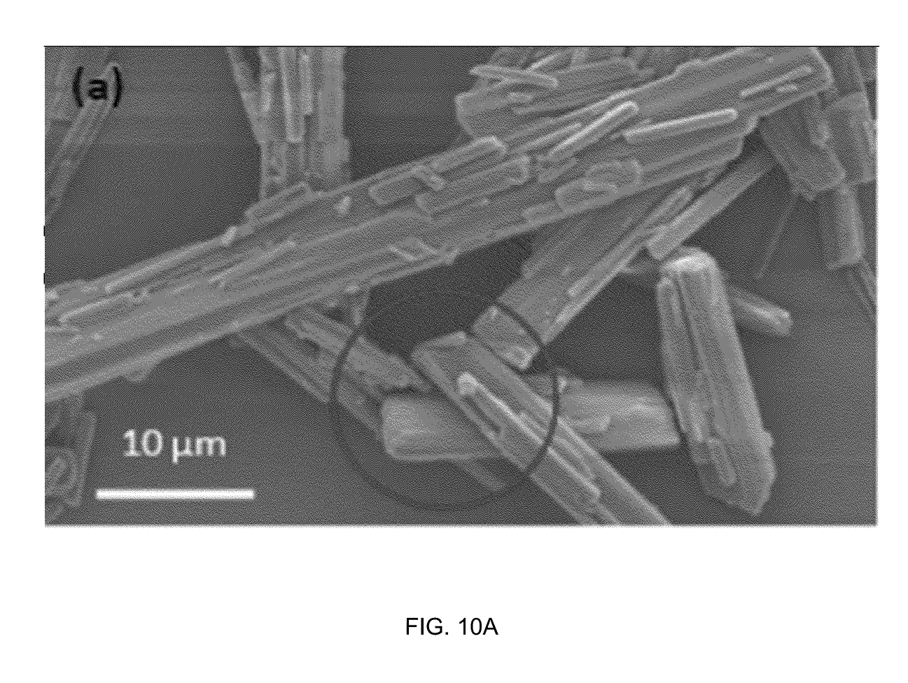

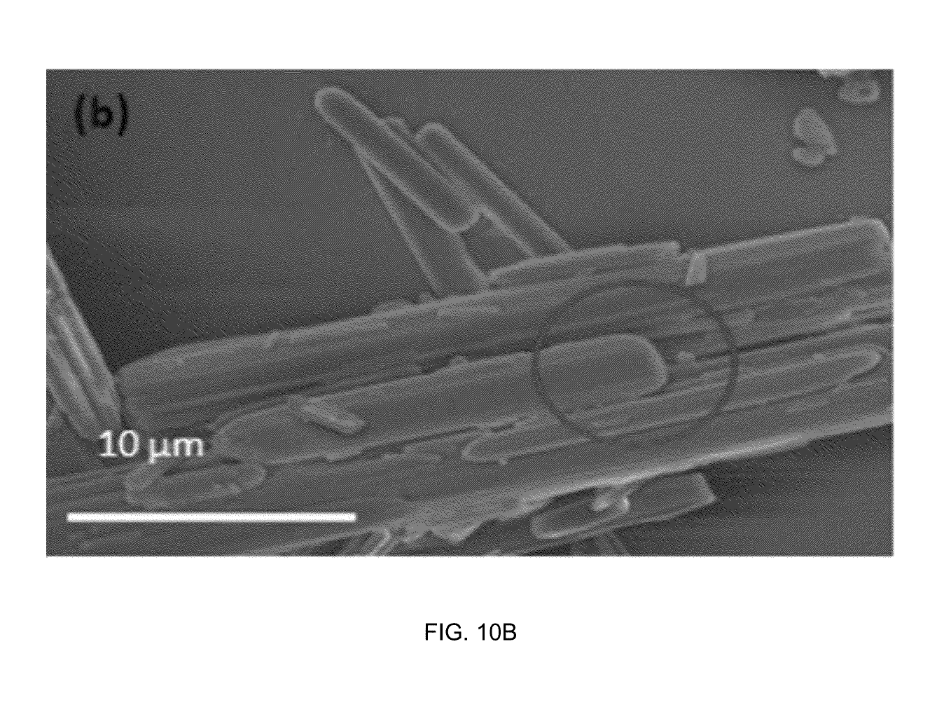

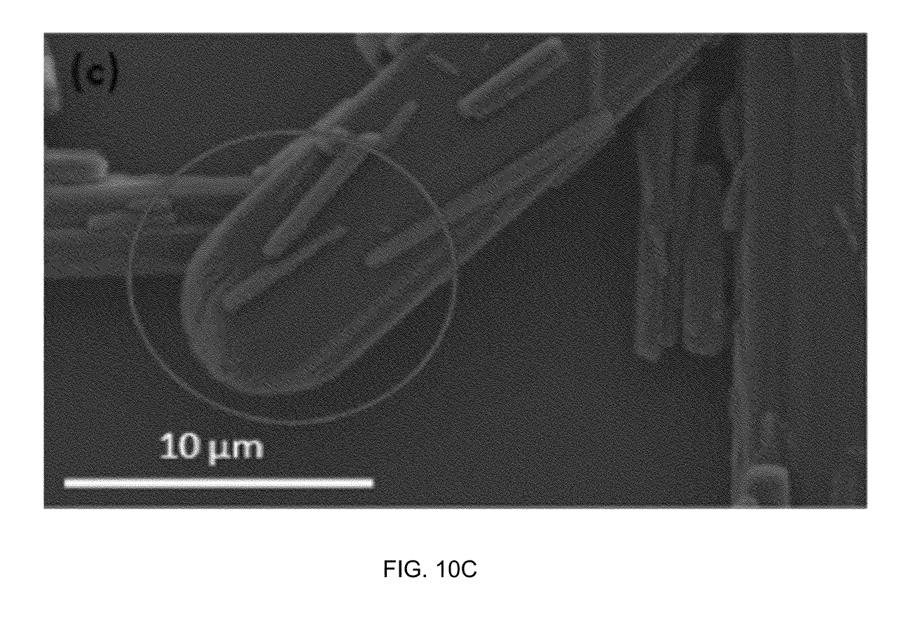

Therefore, in order to achieve an increase in bioavailability, the polymer contained in a drug formulation should not only improve dissolution rate, but also increase drug solubility, by inhibiting drug crystallization. While CAP 504-0.2 Adp appears to be an excellent crystallization inhibitor, when formulated as a solid dispersion, it suppresses the solution concentrations achieved by retarding dissolution rate. This shortcoming of CAP 504-0.2 Adp was resolved by combining it with a hydrophilic polymer, PVP. A solid dispersion of ritonavir, containing PVP and CAP 504-0.2 Adp in a ratio of 10% drug, 80% PVP and 10% CAP 504-0.2 Adp was prepared. This binary combination of polymers in a solid dispersion merges the superior properties of the two polymers: the crystallization inhibitory characteristic of CAP 504-0.2 Adp and the ability of PVP to increase dissolution rate of ritonavir. As illustrated in FIG. 9, with this amorphous solid dispersion, not only is ritonavir readily released into solution, but the duration of supersaturation is also prolonged. Further, FIGS. 10A-C provide SEM micrographs of ritonavir seed crystals: (A) before and (B) after crystal growth experiment in the absence of polymer at an initial concentration of 10 mg mL⁻¹ (σ=2.0) (C) after crystal growth experiments in the absence of polymer at an initial concentration of 20 mg mL⁻¹ (σ=2.7). As shown qualitatively, the ends of the needles change from having flat edges to being rounded, which is a characteristic of rough growth after growth experiment at σ>σ_{c}.

### Example II

### Ellagic Acid Compositions

Ellagic Acid (EA):

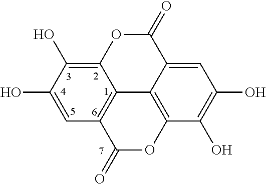 [SMILES: O=c1oc2c(O)c(O)cc3c(=O)oc4c(O)c(O)cc1c4c23]

is a polyphenolic flavonoid present in many dietary sources including walnuts, pomegranates, strawberries, blackberries, cloudberries and raspberries. It has been found that EA has important beneficial health effects against many oxidation-linked chronic diseases. Among the most important examples are cancer, including breast cancer, prostate cancer, lung cancer, and colon cancer, cardiovascular disease, and neurodegenerative diseases. The poor oral bioavailability of ellagic acid, however, is a great challenge for the study of its beneficial functions, making it difficult to translate in vitro results into in vivo studies. In addition, it has been estimated that the average individual consumes approximately 343 mg EA per year, which is not enough to reach the plasma levels required for lung cancer prevention, given its low bioavailability. Poor EA aqueous solubility (9.3 μg/ml at pH 7.4) is a primary cause for its low bioavailability. This low solubility is due in part to its high degree of crystallinity (melting point not observed due to decomposition at about 360° C.), which is a direct result of the planar and symmetrical EA structure and the extensive hydrogen-bonding (H-bonding) network formed in the crystal. In order to develop its therapeutic potential, it is necessary to develop delivery systems that enhance EA solubility, stability and bioavailability.

Preparation of ellagic acid compositions of the invention. Ellagic acid dihydrate (97%) was purchased from Alfa Aesar (Ward Hill, Mass.). PVP (K29-32, Mw 58,000) and potassium bromide (99+%, for spectroscopy, IR grade) was supplied by Acros Organics (Geel, Belgium). CMCAB (641-0.2) was obtained from Eastman Chemical. CAAdP (DS(acetyl)=0.04, DS(propionyl)=2.09, DS(adipate)=0.33) was synthesized by procedures described herein. HPMCAS (AS-LG) was supplied by Shin-Etsu Chemical Co., Ltd. (Tokyo, Japan). Acetone (HPLC grade, 0.2 μm filtered), reagent ethanol, potassium phosphate monobasic, and sodium hydroxide were supplied by Fisher Scientific (Fair Lawn, N.J.). Buffer solutions (pH 6.8 and 1.2) were prepared according to USP30-NF25 standard method.

Preparation of spray-dried solid dispersions. Mixtures of EA/polymer (PVP, CMCAB and HPMCAS) (10.0 g) at different weight ratios (1/9 and 1/3) were dissolved in 500 mL of acetone/ethanol (1/4, v/v) to make feed solutions. Solid dispersions were prepared using a Buchi mini-spray dryer B-290. Operating parameters were: inlet temperature, 90° C.; outlet temperature, 57-60° C.; feed rate, 9 mL/min; nitrogen flow 350 L/h. Yields of the spray-drying process were 50-60%.

Co-precipitated solid dispersions. It was convenient to prepare EA/CAAdP dispersions by co-precipitation due to the limited quantity of CAAdP available, and the unavoidable losses incurred when spray-drying small quantities of solid dispersions. A mixture of EA/CAAdP (1/3 or 1/9) (0.2 g) was dissolved in 10 mL of THF. Then the solution was added dropwise to 200 mL of DI water with stirring. The precipitate was collected by filtration, then dried under vacuum at 40° C. overnight. The drug content was confirmed by UV-vis spectrometry.

ASD by rotary evaporation. It was convenient to prepare the EA/PVP/CAAdP dispersion by rotary evaporation due to the limited quantities of CAAdP available and the high water solubility of PVP. EA (20 mg), PVP (90 mg) and CAAdP (90 mg) were dissolved in acetonitrile/EtOH (1/1, v/v; 40 mL). The solution was concentrated by rotary evaporation. The residue was dried under vacuum at 40° C. overnight. Physical mixtures were prepared to compare to the spray-dried samples by grinding weighed portions of EA and HPMCAS, CAAdP, CMCAB or PVP with a mortar and pestle.

Characterization of EA/matrix solid dispersions. EA/polymer solid dispersions were characterized by comparing FTIR and NMR spectra, DSC traces, and XRPD patterns obtained for EA, the pure individual polymers, physical mixtures of EA/polymer, and EA/polymer solid dispersions. FTIR spectra were recorded in a frequency range between 4000 and 400 cm-1, using a resolution of 4 cm-1 and 40 accumulations, on a Nicolet 8700 FT-IR spectrometer. FTIR pellets comprised 1 mg of the polymer matrix mixture and 100 mg of potassium bromide.

NMR spectroscopy. Solid-state CP MAS ¹³C-NMR experiments were performed on a BRUKER Avance II 300 spectrometer at 75.47 MHz, equipped with a MAS probe head using 4 mm ZrO₂ rotors. Glycine was used to set the Hartmann-Hahn conditions and adamantane as secondary chemical shift reference δ=38.48 ppm and 29.46 ppm from external TMS, respectively. Solution ¹³C-NMR spectra were recorded with a proton 90° pulse length of 4.0 μs and a contact time of 1 ms. Repetition delay was 10 s, spin rate 7 k, number of scans 512 within 1.5 h, and spectral width 25 kHz. FIDs were accumulated with a time domain size of 1 K data points. RAMP shape pulse was used during the cross-polarization and spinal64 for decoupling during acquisition. Spectral data were processed using the Topspin program.

XRPD analysis XRPD measurements used a Bruker D8 Discovery X-ray diffractometer. Measurements were performed at a voltage of 40 kV and 25 mA. The scanned angle was set as 5<2θ<40° and the scan rate was 2°/min.

DSC measurement. EA and solid dispersions were analyzed using a modulated differential scanning calorimeter (Model Q2000, TA Instruments, New Castle, Del.) equipped with a refrigerated cooling accessory. Samples (4-5 mg) were packed in non-hermetically crimped aluminum pans, heated under dry nitrogen from 25 to 100-120° C. at 10° C./min to eliminate moisture and relieve stress, then quickly cooled to 25° C. at 100° C./min. Samples were then heated to 200° C. at 3° C./min with ±1° C. modulation every 45 s; glass transitions are reported from this second heating scan based on the reversible heat flow. DSC heating curves were analyzed using Universal Analysis 2000 software (TA Instruments).

UV-vis spectroscopy. All UV-vis spectra were recorded on a Thermo Scientific Evolution 300 UV-Visible Spectrometer.

Measurement of matrix polymer solubility. Polymer (0.5 g; CMCAB, HPMCAS, CAAdP, or PVP) was dispersed in 10 mL of pH 6.8 buffer. The suspension was mixed by a vortex mixer for 1 min, ultrasonicated for 15 min, and then shaken for 24 h at room temperature (Burrell wrist action shaker, Model 75). The suspension/solution was centrifuged at 14,000×g for 10 min to remove insoluble material. An aliquot (1 mL) of the top, clear solution was withdrawn and the solvent evaporated in an oven (80° C., 5 h). The dissolved polymer weight was calculated by subtracting the weight of salt in buffer solution (7.2±0.1 mg/mL). The dissolved polymer concentration (w/v) was then calculated by dividing the dissolved polymer weight by the volume of solution withdrawn.

Ellagic acid calibration curves in N-methylpyrrolidone (NMP) and pH 6.8 buffer. Careful attention is needed in construction of a practical and appropriate calibration curve that covers supersaturated concentrations of a poorly soluble species like EA. From the standard curves of EA (see Supplementary Material S3), the extinction coefficient of EA is quite similar in NMP (38.3 L g⁻¹ cm⁻¹) and in NMP/pH 6.8 buffer solution (1/99, v/v; 39.5 L g⁻¹ cm⁻¹). In creating appropriate calibration curves, one must also keep in mind the ionization state of a molecule like EA that possesses multiple weakly acidic phenol groups. EA UV/vis calibration curves were used in NMP for experiments at pH 1.2, and calibration curves in NMP/pH 6.8 buffer (1/99, v/v) for experiments at pH 6.8. The EA standard curve in NMP was used for the calculation of concentration from UV-vis absorption at pH 1.2 since most EA is not ionized at that pH. Calibration curves in aqueous buffer (pH 6.8) were generated by dilution of an EA stock solution in NMP (2.5 mg/mL) with pH 6.8 buffer solution to 10 mL (fixing the ratio of NMP/pH 6.8 buffer 1/99, v/v).

Dissolution testing. EA solid dispersion (EA content fixed at 50 mg) was dispersed in 10 mL of pH 6.8 phosphate buffer in an amber flask with magnetic stirring for 24 h. Then the suspension was centrifuged (14,000×g, 10 min) to remove insoluble material. EA concentration in the supernatant was determined by UV-vis spectrometry using the calibration curve in pH 6.8 buffer generated as described above.

Enhancement of ellagic acid stability. Stability enhancement of EA by polymers in solution was studied by following decline in EA solution concentration in the presence or absence of polymer, using UV-vis spectrometry. EA and EA/PVP (1/9) solid dispersion samples were dissolved in ethanol, while EA/cellulose ester (1/9) solid dispersions were dissolved in THF due to the low solubility of cellulose derivatives in ethanol. EA concentration was fixed at 0.2 mg/mL. Samples (1 mL) of each stock solution were diluted to 10 mL with pH 6.8 buffer. The amount of EA still in solution was measured by UV-vis absorption of the diluted solution at time intervals from 0.5 to 24 h. EA chemical degradation in aqueous buffer was studied as follows. Samples of EA or EA/polymer 1/9 solid dispersion were dissolved in ethanol or THF ([EA]=0.2 mg/mL), then 200 μl aliquots of each solution were added to pH 6.8 aqueous buffer (800 μL). Samples were incubated at room temperature for the indicated time. After incubation, each mixture was diluted by 1 mL of ethanol and the UV-vis absorption of the diluted solution was measured.

Ellagic acid release profile. EA samples (pure, physical mixture or solid dispersion) were dispersed in 100 mL pH 6.8 buffer in an amber glass flask in amounts that provided in each case an EA concentration of 0.05 mg/mL. The solution was stirred with a stir bar at 25° C. Aliquots (1.5 mL) were withdrawn at appropriate time intervals and replaced with 1.5 mL of fresh dissolution medium after each sampling to maintain constant volume. UV-vis absorption of each aliquot was recorded after centrifugation (14,000×g, 10 min). Release profiles in pH 1.2 buffer were measured using the same method and the aliquots were centrifuged before UV-vis measurement. The first three time points (0.1, 1.3, 2.7 min) from EA/PVP 1/9 solid dispersions were measured directly without centrifugation, because of the rapid initial release from those dispersions and the time required for centrifugation.

Dissolution testing and solution concentration enhancement. Maximum solution concentration from pure EA and its ASDs at pH 6.8 was measured by UV-vis spectrometry. Such studies can be plagued by UV absorption by nanoparticles that can result from partial crystallization from such supersaturated solutions. This issue was dealt with by centrifugation of samples prior to UV-vis measurements. This protocol gave highly repeatable values with a standard deviation of less than 5% for three duplicates. In the dissolution tests, EA solid dispersions were dispersed in pH 6.8 buffer solution. Aliquots were removed, centrifuged and analyzed at various time points. Equilibrium was usually reached (as determined by [EA] plateau) within 1-5 h. PVP and HPMCAS molecular dispersions with lower EA content led to higher EA solution concentrations. EA solution concentration obtained from solid dispersions depends strongly on polymer structure in the following sequence PVP>HPMCAS>CMCAB, which corresponds with the relative aqueous solubility of the three polymers (Table 5).

TABLE 5
Solubility of polymers and maximum EA concentration from their ASDs.

| | Solubility | Maximum EA concentration |
|---|---|---|
| Polymer | in water (mg/mL) | from amorphous dispersion (μg/mL) |
| PVP | [symbol] 600 | 1500 |
| HPMCAS | 23.4 | 280 |
| CMCAB | 1.6 | 30 |
| CAAdP | 1.5 | Not measured |

This relationship may be a result of the fact that more hydrophilic polymer matrices swell or dissolve more rapidly in aqueous buffer, affording faster release kinetics. Increased EA solution concentration could also result from the higher polymer solution concentrations observed with PVP and HPMCAS; the increased amounts of dissolved polymer may increase thermodynamic solubility of EA, or may more effectively inhibit EA crystallization and degradation.

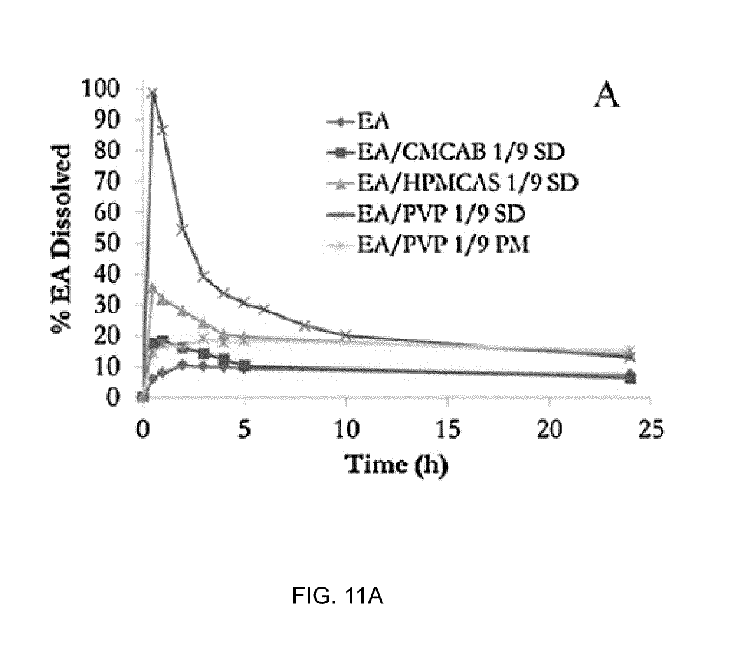

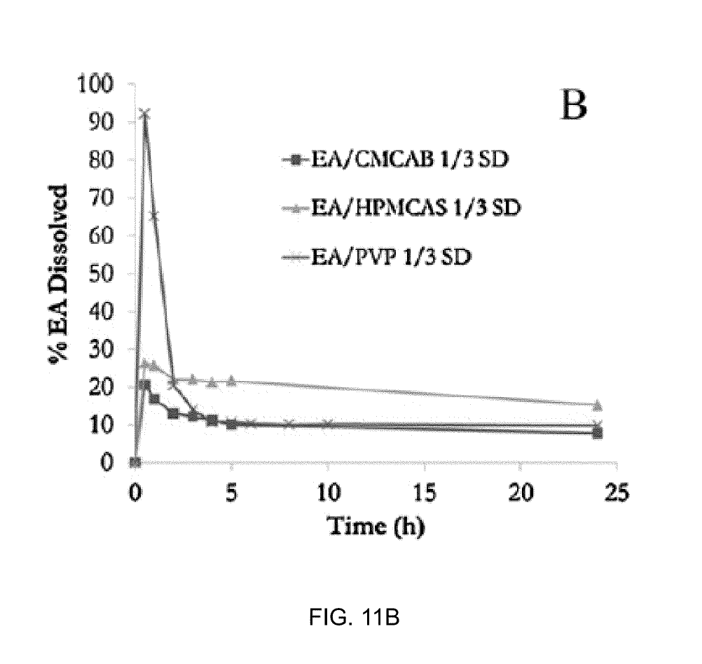

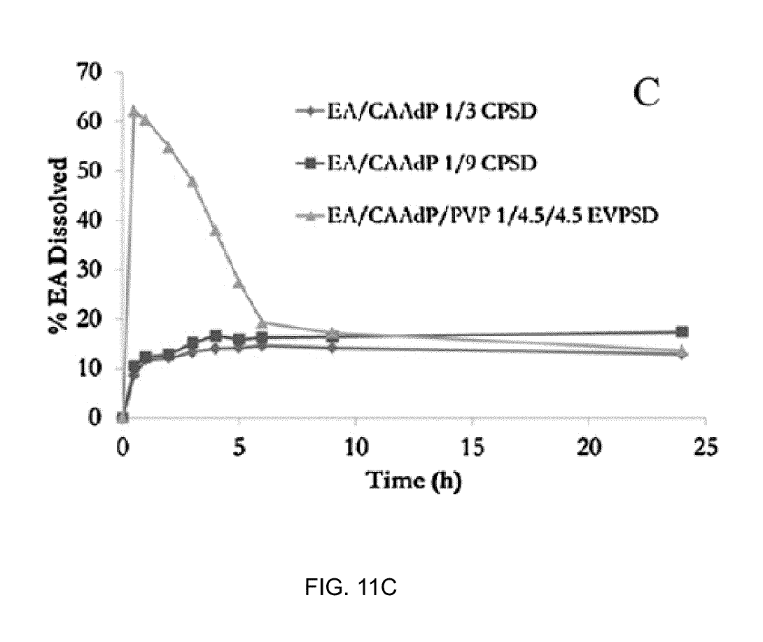

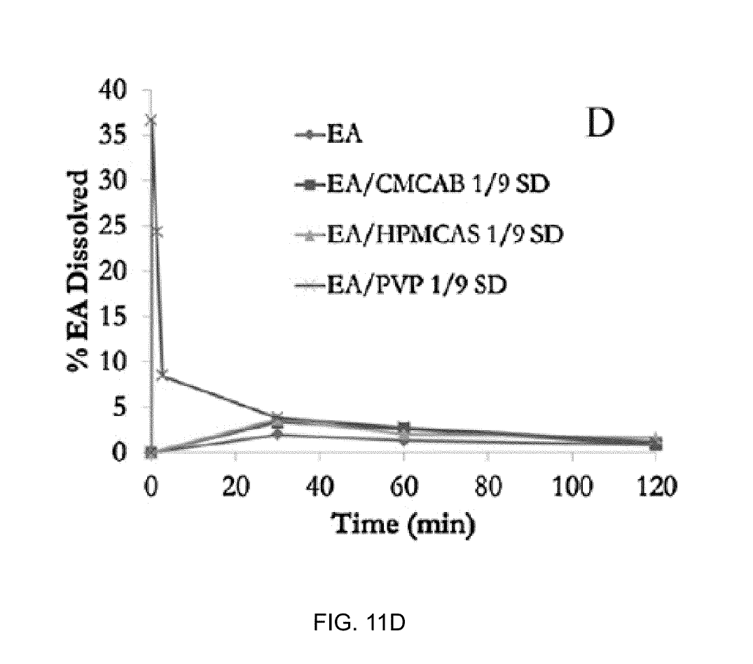

Drug release profiles. Dissolution of EA from ASDs was compared with that of pure EA (5 mg) and that from EA/polymer physical mixtures, both at pH 6.8 and 1.2. Nanoparticle removal from sample aliquots was effected by centrifugation (14,000×g). UV-vis absorption of the solution was measured and plotted vs. time. The influence of polymer type and of EA/polymer ratio on drug release profiles was also investigated. Drug release profiles of EA, EA/PVP 1/9 physical mixture, and EA/polymer (CMCAB, CAAdP, HPMCAS and PVP) ASDs in pH 6.8 buffer are shown in FIGS. 11A-D.

More specifically, FIGS. 11A-D are graphs showing dissolution from: (A) EA, EA/PVP 1/9 physical mixture, EA/polymer 1/9 solid dispersions (pH 6.8, UV-vis); (B) EA/polymer 1/3 solid dispersions (pH 6.8, UV-vis); (C) EA/CAAdP (1/3, 1/9) co-precipitating solid dispersions (CPSD) and EA/CAAdP/PVP (1/4.5/4.5) evaporation solid dispersion (EVSD), after centrifugation at 14,000×g for 10 min (pH 6.8, UV-vis); and (D) dissolution of EA and EA/polymer 1/9 ASDs (pH 1.2, UV-vis). As shown, release from the EA/PVP 1/9 solid dispersion was fastest and most complete (FIG. 11A), reaching 92% within 1 h, while release from the EA/HPMCAS blend (1/9 SD) was much slower and incomplete, reaching a maximum of 35% after 0.5 h. Release from the EA/CMCAB 1/9 SD was slowest, reaching a maximum of 18% after 1 h. EA concentration in each case decreased from a maximum value, presumably due to incomplete stabilization of supersaturated EA concentrations by the polymers. After 24 h, the drug release yield from EA/PVP and EA/HPMCAS 1/9 SDs decreased to 13-14%, and from EA/CMCAB 1/9 SD was only 6%. Release from the EA/PVP 1/9 physical mixture was much slower and less complete than that from the ASD of the same composition, reaching a maximum of 19% EA released at 3 h, where a similar relative comparison of HPMCAS 1/9 ASD with the HPMCAS physical mixture of equal composition can be seen. After 24 h the amount of EA in solution from PVP 1/9 ASD and physical mixture is the same; clearly at long dissolution times the recrystallization of EA, and the failure of these polymers to completely prevent it, becomes an important factor. However the higher extent of release from PVP ASD in the early part of the curve means that there would be much more chance of EA absorption, and presumably much higher bioavailability, from the ASD than from the physical mixture. Dissolution from both PVP and HPMCAS ASDs is far superior to that of pure EA, which reaches a maximum of 11% at 2 h, then decreases to 7% at 24 h. Dissolution from the CMCAB ASDs is slow, similar to that from pure EA. This is believed to be due largely to poor release of hydrophobic EA from the hydrophobic CMCAB matrix. This analysis is supported by the reasonable ability of CMCAB to stabilize EA against crystallization and degradation once it is in solution. Overall it can be seen that supersaturated solutions of EA can be achieved from HPMCAS and PVP ASDs, but that even these polymers retard, but do not stop crystallization of the highly symmetrical EA from supersaturated solutions. Thus, EA/polymer ratio is a factor impacting extent of drug release.

Release from EA/PVP 1/3 ASDs (FIG. 11B) reaches a maximum of 76%, decreasing rapidly to 17% within 2 h. Compared with the release profile from EA/PVP 1/9 SD (FIG. 11A), the 1/3 ASD affords lower maximum EA release and much faster decrease in EA concentration after the peak. Thus the release rate and the inhibition of EA crystallization strongly depend on the concentration of the PVP ASD. The slower drug release from EA/HPMCAS and EA/CMCAB 1/3 and 1/9 ASDs appears to be somewhat less dependent on EA concentration in the ASD.

CAAdP solid dispersions show drug release profiles similar to those of pure EA and EA/PVP 1/9 PM (FIG. 11C). The highest drug release from CAAdP ASDs is around 15-17%, similar to that of CMCAB ASDs. Since CAAdP and CMCAB have similar solubility in pH 6.8 aqueous buffer, it is not surprising that their ASDs show similar drug release profiles. To improve CAAdP drug release properties, an interesting experiment was conducted in which a combination of PVP and CAAdP (1/1, w/w) was used to prepare an ASD blend with 10% EA content by rotary evaporation. The release profile of EA/CAAdP/PVP (1/4.5/4.5) ASD is similar to EA/PVP 1/9 ASD except that the maximum release at 0.5 h is somewhat lower (62%). EA release from 1 to 5 h is higher than that from EA/PVP 1/3 SD, but lower than that from EA/PVP 1/9 SD, which is reasonable since the EA/PVP ratio is 1/4.5 in this solid dispersion. This experiment highlights the potential of properly designed ASD polymer blends for achieving all requirements of a functional ASD formulation.

It was enlightening to compare EA release from these solid dispersions under conditions similar to those of the stomach, in pH 1.2 buffer (FIG. 11D). Release from the PVP amorphous blend (EA/PVP 1/9 SD) was quite fast but the percentage of dissolved EA decreased very quickly, reaching 37% within a few seconds (the peak release is so early and decline from the peak is so rapid that it is difficult to sample quickly enough; the actual peak could be even higher). In contrast, release was very slow from EA amorphous dispersions with CMCAB and HPMCAS. These results are consistent with the slightly basic nature of PVP and the acidic nature of the carboxyl-containing cellulose esters. Low pH release from the three cellulose ester ASDs is minimal and similar to that from pure EA and from the physical mixtures. In contrast, a substantial portion of EA is released from the PVP ASD, then recrystallizes at pH 1.2. This suggests that although EA/PVP 1/9 ASD affords the best EA release at pH 6.8 among the ASDs studied, this advantage would not be observed in the human GI tract. EA would be largely released and recrystallized in the stomach, and would no longer be part of an ASD by the time it reached the small intestine. In contrast, the cellulose derivatives can retain EA in the amorphous dispersion in the stomach, and properly designed and selected cellulose-carboxyalkanoate polymers, and blends thereof with more water-soluble polymers, would provide effective drug release in the small intestine.

### Example III

### Resveratrol Compositions

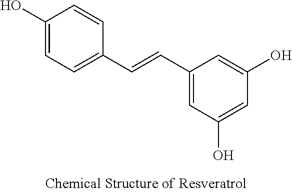 [SMILES: Oc1ccc(/C=C/c2cc(O)cc(O)c2)cc1]

Resveratrol (Chemical Abstracts Service Registry Number CAS 501-36-0) is a phytochemical of great current interest. The compound is found in nature as both cis and trans isomers, however, the trans isomer is believed to be the most abundant and biologically active form. Resveratrol has been suggested to possess antiplatelet, antioxidative, antifungal, anticancer, and cardioprotective properties. In addition, resveratrol has been shown to increase the life span in several species including yeast cells by acting as a calorie restrictor by stimulating SIRT1-dependent deacetylation of p53. Resveratrol is moderately hydrophobic (log P 3.1) but has poor aqueous solubility, in large part due to its high melting point of 262° C. and strong crystal lattice energy. Consequently, amorphous formulation of this compound is of great interest, as substantial improvements in dissolution rate and transient solubility should be achieved. Resveratrol is a particularly interesting model compound because of its extremely high inherent tendency to crystallize. Thus, the inventors studied the impact of polymer type and functionality on the formation and storage stability of amorphous resveratrol. Model polymers included poly (vinylpyrrolidone) (PVP) K29/32, PVP K-12, hydroxypropyl methylcellulose (HPMC), hydroxypropyl methylcellulose acetate succinate (HPMCAS), poly (acrylic acid) (PAA), carboxymethyl cellulose acetate butyrate (CMCAB), and Eudragit® E100 (E100).

Solutions were prepared by dissolving both resveratrol and polymer at different dry weight ratios (from 5% to 75% resveratrol in 5% increments) in a 1:1 (by weight) mixture of dichloromethane and ethanol. All mixtures were visually inspected to confirm that the resveratrol and the polymers were fully dissolved, and that the systems formed uniform one-phase solutions. The solvent was removed using a rotary evaporator (Brinkman Instruments, Westbury, N.Y.) in a water bath maintained at 60° C. The samples were then placed under vacuum for 24 h to remove any residual solvents. The obtained material was subsequently cryomilled in a liquid nitrogen bath for a total milling time of 4 min.

Selected samples were stored at five different environmental conditions: room temperature (22° C.-25° C.) in a desiccator filled with P₂O₅ [0% relative humidity (RH)], room temperature (22° C.-25° C.) in a desiccators filled with KCl (84% RH), as well as 50° C., 90° C., and 120° C. in desiccators filled with Drierite™ (W.A. Hammond Drierite Company, LTD, Xenia, Ohio, USA, anhydrous calcium sulfate, 0% RH) for up to 400 days and sampled periodically.

Powder X-Ray Diffraction. Immediately after cryomilling, the samples were analyzed using a Shimadzu XRD-6000 (Shimadzu Corporation, Kyoto, Japan) equipped with a Cu-Kα source and set in Bragg-Brentano geometry between 5° and 35° 2θ at 8°/min with a 0.04° step size. Samples were also analyzed over time following the storage treatments listed above. Before each day of measurement, the accuracy of the 2θ angle was checked by verifying that the [111] peak of a Si-standard sample was between 28.423° and 28.463°. The measured photon intensity of the [111] peak was used to normalize the data collected from samples on that day. Samples where sharp peaks were observed were deemed crystalline, and those samples that lacked sharp peaks were labeled X-ray amorphous. Physical mixtures of select resveratrol-polymer systems were made to provide a fully crystallized reference. The percent crystallized for certain samples was estimated from a calibration curve generated from analysis of the diffraction patterns of the different solid dispersions, spiked with increasing levels of crystalline resveratrol. The detection limit was found to be of the order of 5%-10% crystalline material. The crystallinity of these calibration samples was measured immediately after preparation.

Infrared Spectroscopy. Solutions were prepared by dissolving both resveratrol and polymer at different dry weight ratios in a 1:1 (by weight, ratios ranged from 5% to 60% resveratrol) mixture of dichloromethane and ethanol. All mixtures were visually inspected to confirm that the resveratrol and the polymers were fully dissolved, and that the systems formed uniform one-phase solutions.

Two or three drops of the solutions were placed on thallium bromoiodide (KRS-5) optical crystals, which were immediately rotated on a KW-4A spin coater at 500/2500 rpm for 18 s and 30 s, respectively. Infrared (IR) spectra of the resulting thin films were obtained in absorbance mode using a Bio-Rad FTS 6000 spectrophotometer (Bio-Rad Laboratories, Hercules, Calif.) equipped with globar IR source, KBr beamsplitter, and DTGS detector. The scan range was set from 4000 to 500 cm⁻¹ with 4 cm⁻¹ resolution, and 128 scans were co-added. The absorbance intensity of the spectral region of interest was between 0.6 and 1.2. During measurements, the spin-coated samples and the sample compartment of the spectrophotometer were flushed with dry air.

Differential Scanning Calorimetry. Thermal analysis was performed using a TA Q2000 DSC equipped with a refrigerated cooling accessory (TA Instruments, New Castle, Del.). Nitrogen, 50 mL/min, served as the purge gas. Samples (3-7 mg) were weighed into aluminum Tzero sample pans with pinholes (TA instruments) and sealed. Samples were heated from 0° C. to 120° C. at a heating rate of 10° C./min, then quickly cooled to 0° C. at the maximum instrument cooling rate (15° C./min), then heated from 0° C. to 200° C. at a heating rate of 10° C./min; transitions are reported from this second heating scan. For the PVP solid dispersions, the Tg could not be resolved without modulation. These samples were heated from 25° C. to 120° C. at a heating rate of 10° C./min then quickly cooled to 0° C. at the maximum cooling rate of the instrument, held isothermally at 0° C. for 5 min, then heated at 2° C./min to 200° C. with a modulation of ±1° C. every 60 s. Differential scanning calorimetry (DSC) heating curves were analyzed using Universal Analysis 2000 software (TA Instruments). Tg values of samples at 84% RH were obtained by weighing the pre-equilibrated sample into an aluminum Tzero pan followed by hermetic sealing. The sample was cooled to −50° C. and subsequently heated at a rate of 10° C./min.

Moisture Sorption Isotherms. Moisture sorption analysis was conducted using a Thermal Gravimetric Analyzer Q5000 (TA Instruments). Samples (5-10 mg) were dried at 50° C. using an equilibrium criterion of 0.01% (w/w) with a maximum drying time of 360 min, cooled to 25° C., and then exposed to increasing RHs from 0% RH to 95% RH with a step rate of 5% RH at 25° C. using an equilibrium criterion of 0.01% (w/w) within 5 min or a maximum equilibration time at each RH of 360 min. Typical runs for solid dispersions took 12 h.

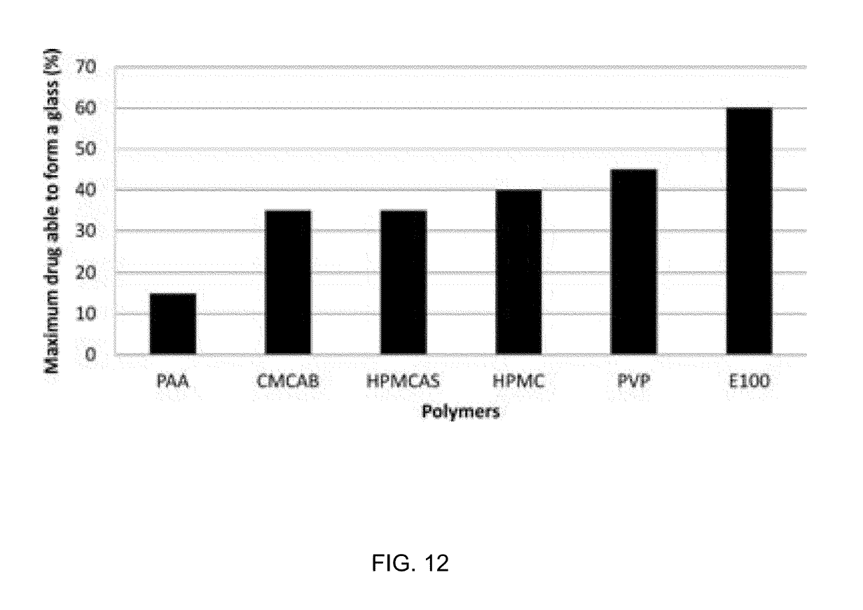

Crystallization Tendency of Resveratrol During Production. All attempts to prepare amorphous resveratrol using the methods described above failed. This means that resveratrol can be classified as a rapid crystallizer according to the classification system of Van Eerdenbrugh et al. See Van Eerdenbrugh B, Baird J A, Taylor L S. 2010, “Crystallization tendency of active pharmaceutical ingredients following rapid solvent evaporation-classification and comparison with crystallization tendency from undercooled melts,” J Pharm Sci 99(9):3826-3838. On the basis of these observations, it is clear that to prepare stable, amorphous resveratrol, a crystallization inhibitor is necessary. Polymers proved to be effective crystallization inhibitors for amorphous resveratrol; however, the polymers showed very different inhibitory abilities. E100 stood out as the best crystallization inhibitor, requiring a relatively small amount of polymer to produce the solid dispersions during solvent evaporation, whereas PAA required so much polymer that there was very little resveratrol in the dispersion. These results are summarized in FIG. 12, where the highest percentage of resveratrol in the polymer-resveratrol solid dispersion that leads to an X-ray amorphous system following rotary evaporation is displayed. In the resveratrol systems prepared by rotary evaporation, 40% E100, 55% PVP K29/32, 60% HPMC, 65% HPMCAS, 65% CMCAB, and 85% PAA were required to yield an amorphous sample. When using a faster solvent evaporation technique, spin coating, consistently less polymer was required to produce an amorphous sample; however, the order of polymer effectiveness remained constant.

### Example IV

### Compositions with Binary Additive Combinations

The associations between combinations of polymers and those of polymer/surfactant systems in aqueous solution have garnered considerable fundamental and technological interest. The species resulting from the interactions of these molecules possess unique properties that differ from those of the individual components. As a result, they have important applications in industrial and biological processes. For example, polymer and surfactant combinations have been used for rheology control and immobilization of enzymes in polyelectrolyte complexes, while synthetic water-soluble polymer combinations have been used in mineral-processing operations such as flocculation. In the drug delivery field, polymers and surfactants are often incorporated in formulations to modify the solubility of drugs with low aqueous solubility and to control the release rate. Thus, associations between polymers and surfactants are highly relevant, since pharmaceutical formulations often contain both component types. Formulations containing the drug as a high-energy amorphous solid can enhance drug delivery by generating supersaturated solutions. Polymers are typically incorporated to delay drug crystallization, and surfactants are frequently added to improve processing properties or dissolution profiles.

The equilibrium solubility of ritonavir was determined in the absence and presence of selected additives (polymers and surfactants). Prior to adding an excess amount of ritonavir to 100 mM sodium phosphate buffer, pH 6.8, additives were predissolved in the buffer at a concentration of 5 μg/mL. The drug-additive solution was equilibrated at 37° C. for 48 h. Using ultracentrifugation, the supernatant was separated from the excess solid in solution. Ultracentrifugation was performed at 40,000 rpm (equivalent of 274,356 g) in an Optima L-100 XP ultracentrifuge equipped with a Swinging-Bucket Rotor SW 41 Ti (Beckman Coulter, Inc., Brea, Calif.). The supernatant was diluted using a combination of mobile phase solvents. Solution concentration was determined using an Agilent 1100 high performance liquid chromatography (HPLC) system (Agilent Technologies, Santa Clara, Calif.). Ritonavir was detected by ultraviolet (UV) absorbance at a wavelength of 240 nm. The chromatographic separation was performed with a Zobrax SB-C18 analytical column (150 mm×2.1 mm i.d., 5 μm, 100 Å) (Agilent Technologies, Santa Clara, Calif.). A mixture of sodium phosphate buffer (10 mM, pH 6.8) (40%) and acetonitrile (60%) was used as mobile phase, and mobile phase flow was maintained at 0.2 mL/min. The injection volume was 20 pt. The total analytical run time was 20 min. Ritonavir standards (0.5-20 μg/mL) were prepared in methanol. The standards and samples were analyzed in triplicate. The standard curve exhibited good linearity (r²>0.9995) over the concentration range. The regression intercept for the calibration curve was very small and was not statistically significant compared to zero.

Characterization of Seed Crystals. Ritonavir seed crystals were characterized using scanning electron microscopy (SEM), powder X-ray diffraction analysis (PXRD), and differential scanning calorimetry (DSC). SEM was used to determine the size and shape of ritonavir crystals, while PXRD and DSC were used to evaluate the polymorph of the seed crystals. Ritonavir was used as received from the manufacturer, and prior to use, the seeds were sieved to a size below 250 p.m. Seed crystals used for SEM analysis were prepared by dispersing the seeds in water and equilibrating the water-seed crystal suspension for approximately 24 h. A few drops of the suspension were placed on a glass slide and dried overnight in a vacuum oven at room temperature for approximately 24 h. Before SEM imaging, the glass slides were mounted using double-sticky copper tape and sputter-coated with platinum for 60 s. SEM imaging was performed using an FEI NOVA nanoSEM field emission SEM using an Everhart-Thornley (ET) detector and a through-thelens detector (TLD). The following imaging parameters were used: 5 kV accelerating voltage, approximately 4-5 mm working distance, beam spot size of 3, 30 μm aperture, and 100-10,000× magnifications. The SEM images were analyzed using ImageJ, processing and analysis in Java (National Institutes of Health (NIH)). Several representative samples of seed crystals were obtained before and after crystal growth in the absence and presence of predissolved additive. For each sample, a total of 50 seeds were analyzed to ensure a representative sample, and the average aspect ratio of the crystals was estimated from the average length and width of the crystals.

DSC analysis was performed using a TA Instruments Q2000 instrument (TA Instruments, New Castle, Del.) attached to a refrigerated cooling accessory (RCS) (TA Instruments, New Castle, Del.). Both the DSC and RCS were purged with nitrogen gas. The thermogram of ritonavir was obtained by heating the sample at a rate of 5° C./min and 20° C./min for melting point and glass transition temperature determinations, respectively. Melting point was determined from a first heat scan, while the glass transition temperature was determined from a second heat scan. The temperature range used was 25-150° C. Thermal transitions were viewed and analyzed using the analysis software Universal Analysis 2000 for Windows 2000/XP provided with the instrument.

PXRD patterns were obtained using a Shimadzu XRD 6000 diffractometer (Shimadzu Scientific Instruments, Columbia, Md.). Calibration was performed using a silicon standard that has a characteristic peak at 28.44° 20. Experiments were performed using an accelerating potential of 40 kV and a current of 30 mA. The divergence and scattering slits were set at 1.0°, and the receiving slit was set at 0.3 mm. Diffraction patterns were obtained within a scan range from 5 to 35° 20, with a scanning speed of 4°/min.

Characterization of Polymers. The relative hydrophobicity of the polymers was characterized using solubility parameter (SP) values. SP values were estimated using methods found in the literature. The method is based on group additive constants, and the contribution of a large number of functional groups was evaluated; therefore, it requires only knowledge of the structural formula of the polymer. The solubility parameter can be evaluated using the following:

$$δ = \sqrt{\frac{{\sum }_{i} Δ {e}_{i}}{{\sum }_{i} Δ {v}_{i}}} = \sqrt{\frac{Δ {E}_{V}}{V}}$$

where the Δei and Δvi are the additive atomic and group contributions for the energy of vaporization and molar volume, respectively.

Crystal Growth Rate. The crystal growth rate of ritonavir was characterized by measuring the rate of desupersaturation in the presence of seed crystals. Crystal growth rate experiments were performed in the absence and presence of predissolved additives at initial ritonavir concentrations of 5, 10, and 20 μg/mL; all experiments were performed in triplicate. Additive concentrations of 5 μg/mL were used; in experiments where a combination of additives was used, the concentration of each additive in solution was 5 μg/mL (unless otherwise specified). Crystal growth experiments were performed in a jacketed beaker connected to a digitally controlled temperature water bath. Solubilized ritonavir was prepared by dissolving 200 mg of ritonavir in 50 mL of methanol to make a final stock solution of 4 mg/mL. Supersaturated solutions were generated by adding a small volume (0.25 mL) of predissolved ritonavir in methanol to sodium phosphate buffer, pH 6.8 (50 mL); the volume of methanol in buffer solution (1:200, methanol to buffer solution) did not have an impact on the equilibrium solubility of ritonavir. Prior to addition of solubilized ritonavir, seed crystals (0.010 g) were added to the buffer and allowed to equilibrate at 37° C. Data collection began immediately after generation of supersaturation; the experiment duration was 30 min. The test solution was stirred at a constant speed of 400 rpm using a Cole Parmer (model 50000-20) overhead mixer attached to an axial-flow impeller blade, with a digital mixer controller (Cole Parmer Instrument Co., Niles, Ill.). The rate of desupersaturation of ritonavir was measured using a UV spectrometer coupled to a fiber optic probe, path-length 5 mm (SI Photonics, Tuscon, Ariz.), at a constant temperature of 37° C. Data was acquired at 5 s time intervals within a wavelength range of 200-450 nm. The slope of the concentration versus time curve over the first 2 min of the experiment was taken as the initial crystal growth rate. In order to mitigate particle scattering effects, second derivatives (SIMCA P+ V. 12 software (Umetrics Inc., Umea Sweden)) of the spectra as well as the calibration spectra were taken for the sample data. Calibration solutions (1-30 μg/mL) were prepared in methanol. The standard curve was linear over the above-mentioned concentration range (r²>0.999). Polymer/polymer interactions were further investigated by performing growth experiments in sodium phosphate buffer at two different ionic strengths, 50 and 100 mM.

Characterization of Seed Crystals. SEM analysis was used to quantitatively characterize the seed crystals before and after crystal growth. Ritonavir is known to have five polymorphs; Form II is the most stable and least soluble polymorphic form of ritonavir. The Form II polymorph was the intended starting material for this study. The seed crystals were needle-like, which is the characteristic crystal habit of Form II ritonavir. PXRD and DSC analysis further confirmed the crystalline form of ritonavir as Form II. The predicted powder pattern of ritonavir polymorphic Form II (CSD code: YIGPIO01), obtained from the Cambridge Structural Database (CSD) using the powder pattern prediction tool, is consistent with the measured PXRD pattern. However, the predicted crystal morphology (BFDH morphology prediction tool in Mercury) is more rodlike than the needles observed experimentally. The melting point and glass transition temperature (measured for the cooled melt) of the seed crystals were 121.0 and 50° C., respectively.

The polymorphic form of the seed crystals after the crystal growth experiment was also confirmed to be Form II by PXRD. The average length, width, and aspect ratio of ritonavir seed crystals before and after the crystal growth experiment in the presence of the CAP Adp 0.85, Pn-IPAAmd, and CAP Adp 0.85/Pn-IPAAmd polymer combination at initial S values of 7.6 and 15.4 are summarized in Table 6.

TABLE 6
Ritonavir Seed Crystal Size

| | | | |
|---|---|---|---|
| | | aspect | crystal |
| length (μm) | width (μm) | ratio | morphology^{c} |

Before Crystal Growth

| | | | | |
|---|---|---|---|---|
| RTV^{a} | 44.4 ± 15.2 | 5.8 ± 1.9 | 7.7 | needle-like |

After Crystal Growth (S = 7.6)

| | | | | |
|---|---|---|---|---|
| RTV^{a} | 50.4 ± 18.6 | 5.7 ± 1.4 | 8.8 | needle-like |
| RTV-CAP Adp | 54.0 ± 18.8 | 10.5 ± 4.1 | 5.2 | rodlike |
| 0.85^{b} | | | | |
| RTV-Pn-IPAAmd^{a} | 41.1 ± 16.5 | 9.3 ± 2.5 | 4.4 | rodlike |
| RTV-CAP Adp | 38.5 ± 11.6 | 8.0 ± 1.4 | 4.8 | rodlike |
| 0.85/Pn-IPAAmd | | | | |

After Crystal Growth (S = 15.4)

| | | | | |
|---|---|---|---|---|
| RTV | 50.9 ± 14.4 | 4.2 + 1.2 | 12.1 | needle-like |
| RTV-CAP Adp 0.85 | 40.9 ± 12.0 | 5.4 ± 1.2 | 7.6 | needle-like |
| RTV-Pn-IPAAmd | 41.6 ± 15.1 | 5.6 ± 1.4 | 7.4 | needle-like |
| RTV-CAP Adp | 37.2 ± 10.0 | 5.7 ± 1.2 | 6.5 | needle-like |
| 0.85/Pn-IPAAmd | | | | |

^{a}Values from ref 31.
^{b}Value from ref 38.
^{c}See FIGS. S1-S3 in the Supporting Information for SEM micrographs.

In general, the seeds extracted after crystal growth at S of 15.4 were more elongated compared to the seeds grown at an S of 7.6 in a similar polymer solution. For example, after crystal growth in the absence of polymer, at S values of 7.6 and 15.4, the aspect ratio (length/width) of the seed crystals was 7.7 and 12.1, respectively. The BFDH morphology predictor of Mercury (Cambridge Crystallographic Data Center, version 3.0.1) suggests that the fastest growth direction is along the a-axis (lengthwise direction) with four fast growing faces ((−1, 0, −1), (−1, −1, 0), (−1, 0, 1), (−1, 1,0)) perpendicular to this direction. The presence of CAP Adp 0.85, Pn-IPAAmd, and their combination in solution inhibited the growth in the fastest growth (lengthwise) direction at both supersaturations and resulted in a decrease in the aspect ratio. It has been proposed that the strong crystal growth inhibitory ability of Pn-IPAAmd can be attributed to a combination of the moderate hydrophobicity of the polymer and fortuitous intermolecular hydrogen bonding to the fast growing faces at the solid-liquid interface. The above-mentioned fast growth planes expose functional groups containing electronegative atoms such as carbonyl oxygen and nitrogen atoms from the thiazole functional group of ritonavir which can potentially interact with the amide group of Pn-IPAAmd.

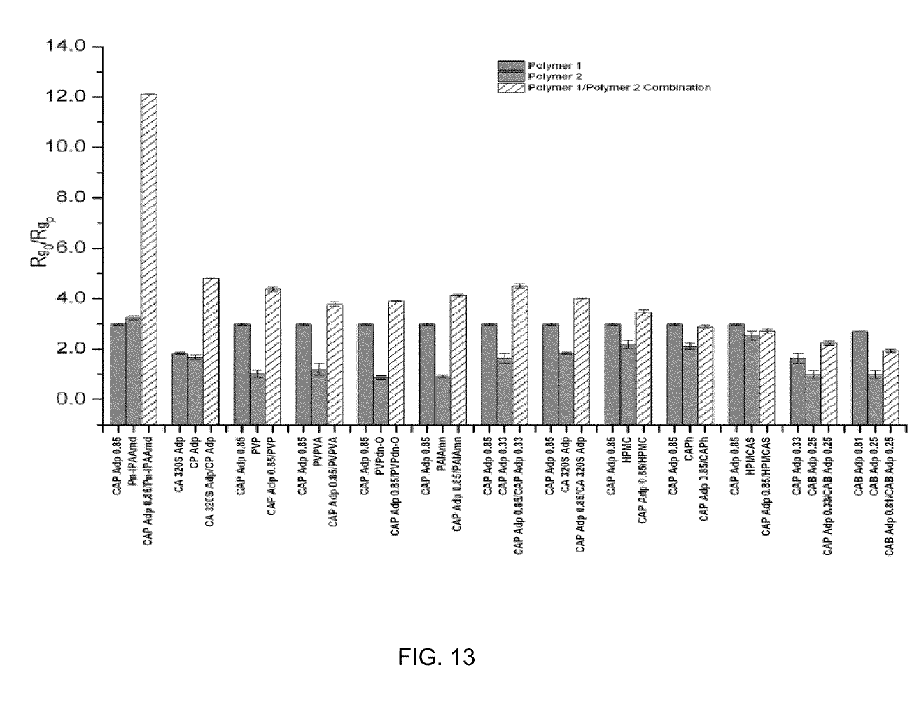

A summary of the growth rate ratios of ritonavir in the absence of polymer (Rg0) to the growth rate of ritonavir in the presence of the polymers/polymer combination (5 μg/mL of each polymer, 1:1 ratio) investigated (Rgp) at an initial concentration of 10 μg/mL (S of 7.6) is shown in FIG. 13. The concentration of each polymer in solution was 5 μg/mL. Crystal growth rate experiments were performed in triplicate. Each column is an average of the effectiveness ratio, and error bars indicate one standard deviation. The y-axis is a ratio of the growth rate of ritonavir in the absence of polymer to the growth rate of ritonavir in the presence of polymer. Polymers with a ratio >1 are considered effective crystal growth inhibitors. The blue and red columns represent the individual polymers, while the white columns with blue diagonal lines represent the polymer/polymer combinations.

The initial bulk crystal growth rate (rate of desupersaturation during the first 2 min) of ritonavir in the 100 mM sodium phosphate buffer at an initial concentration of 10 μg/mL was 1.56 μg mL⁻¹ min⁻¹. At the end of the experiment (30 min), desupersaturation was complete. The red and blue columns represent the individual polymers, while the clear columns with diagonal lines represent the polymer/polymer pairs. Individual polymers and polymer/polymer combinations with an effectiveness ratio greater than 1 (Rg0/Rgp>1) are considered to have some inhibitory ability. Polymers with different structures were employed, of which 5 were synthetic polymers, 3 were commercially available cellulose-based polymers, and 6 were in-house synthesized cellulose derivative polymers with varying physical and chemical properties. Ten out of the 13 combinations that were investigated had a synergistic effect on growth inhibition, that is, the combination of two polymers was more effective in inhibiting crystal growth compared to either of the individual polymers. Five out of the 10 effective polymer pairs were combinations of the adipate cellulose ester, CAP Adp 0.85, previously observed to be one of the most effective cellulose inhibitor studied to date for this system, with various synthetic polymers (e.g., PVP and PVPVA). The CAP Adp 0.85/Pn-IPAAmd polymer combination, which represents the pairing of the two most effective polymers, led to the greatest retardation of growth, where the crystal growth rate was decreased by a factor of 12. In contrast, each individual polymer component of this combination decreased the growth rate by a factor of approximately 3.

Interestingly, combining CAP Adp 0.85 with synthetic polymers that were ineffective when used alone resulted in enhanced growth rate inhibition. For example, although PVP had no impact on ritonavir growth rate alone, when added to CAP Adp 0.85, the ratio of the growth rates increased from approximately 3 to about 4.5. Somewhat surprisingly, combining chemically similar cellulose derivatives also lead to a synergistic effect on crystal growth in some cases with the most effective pairs (CA 320S Adp with CP Adp and CAP Adp 0.85 with CAP Adp 0.33) leading to an increase in the growth rate ratio to about 4.5. The polymer combinations containing CAB Adp 0.25 (CAP Adp 0.33/CAB Adp 0.25 and CAB Adp 0.85/CAB Adp 0.25) were the least effective of all the polymer combinations investigated. In addition, pairing CAP Adp 0.85 with either HPMCAS or CAPh did not inhibit crystal growth any further compared to CAP Adp 0.85 alone.

### Example V

### Celecoxib, Efavirenz, or Ritonavir Compositions

The impact of 21 polymers, added at low levels, on the solution induction times of three drug compounds (celecoxib, efavirenz and ritonavir) was quantified. The polymers that were effective inhibitors were found to have (1) an optimal level of hydrophobicity—the effective polymers had a similar hydrophobicity to the drug molecule, and (2) structure—the cellulose derivatives with bulky side groups were more effective inhibitors compared to the synthetic polymers, even when the level of hydrophobicity of the synthetic polymers was similar to that of the cellulose-based polymers. These factors are likely to affect nucleation by promoting polymer-solute interactions, thereby disrupting the reorganization of a cluster of solute molecules into an ordered crystal structure.

More specifically, the equilibrium solubilities of ritonavir, efavirenz, and celecoxib were determined in the absence and presence of selected polymers. Further, the effectiveness of the polymers for the model compounds was investigated at an initial concentration of 10 μg/mL and this initial concentration corresponds to an initial supersaturation ratio (S) of 6.6 for celecoxib, 1.2 for efavirenz and 7.6 for ritonavir.

The chemical structure for ritonavir is shown above, while the chemical structures for efavirenz and celecoxib are shown below:

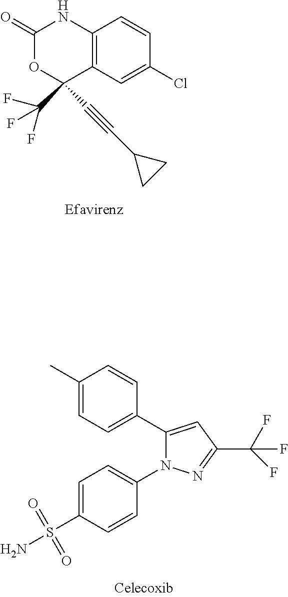 [SMILES: O=C1Nc2ccc(Cl)cc2[C@@](C#CC2CC2)(C(F)(F)F)O1 ; Cc1ccc(-c2cc(C(F)(F)F)nn2-c2ccc(S(N)(=O)=O)cc2)cc1]

Prior to adding an excess amount of crystalline compound to 100 mM sodium phosphate buffer, pH 6.8, the polymer was predissolved in the buffer at a concentration of 5 μg/mL. The drug-polymer solution was equilibrated at 37° C. for 48 h. Using ultracentrifugation, the supernatant was separated from the excess solid in solution. Ultracentrifugation was performed at 40,000 rpm (equivalent of 274,356 g) in an Optima L-100 XP ultracentrifuge equipped with Swinging-Bucket Rotor SW 41 Ti (Beckman Coulter, Inc., Brea, Calif.). The supernatant was diluted using a combination of mobile phase solvents. Solution concentration was determined using an Agilent 1100 high performance liquid chromatography (HPLC) system (Agilent Technologies, Santa Clara, Calif.). Ritonavir was detected by ultraviolet (UV) absorbance detection at a wavelength of 240 nm, while efavirenz and celecoxib were detected at 247 and 249 nm, respectively. The chromatographic separation was performed with a Zobrax SBC18 analytical column (150×2.1 mm I.D., 5 μm, 100 Å) (Agilent Technologies, Santa Clara, Calif.). A mixture of sodium phosphate buffer (10 mM, pH 6.8) (40%) and acetonitrile (60%) was used as mobile phase and mobile phase flow was maintained at 0.2 mL/min. An injection volume of 20 μL was used.

Physicochemical Properties of Model Compounds. The three model compounds, celecoxib, efavirenz, and ritonavir, were selected to cover a range of chemical structures and potential interactions with the polymers. Celecoxib is an aromatic, very weakly acidic molecule (pKa 11.1). Efavirenz is also a very weak acid (pKa 10.2), while ritonavir is a very weak base with two thiazole functional groups with pKa values of 1.8 and 2.6. The model compounds were also selected because they possess intrinsic properties that are expected to result in poor aqueous solubility. Typically, insolubility of pharmaceuticals results primarily from high melting point, Tm (representing lattice energy) and/or high Log P (lipophilicity). Log P and solubility parameter (SP) values were used as indicators of hydrophobicity. Log P values were obtained from the ChemBioDraw Ultra version 12.0 (CambridgeSoft, Cambridge, Mass.) and literature sources, where the values were predicted using atomic contribution methods. The model compounds have different positive Log P values, indicative of varying levels of hydrophobicity. SP values provide a numerical estimate of the cohesive interactions within a material, and can provide an indication of relative polarity. The higher the solubility parameter of a compound, the more hydrophilic it is; water has a SP value of 49.01 MPa^{1/2}. On the basis of Log P and SP values, ritonavir is the most hydrophobic of the model compounds, while celecoxib is the most hydrophilic, although all of the compounds can be characterized as being hydrophobic. Celecoxib had the highest melting point at 163.5° C., while ritonavir had the lowest value of 122.7° C. The equilibrium solubility values of crystalline celecoxib, efavirenz, and ritonavir at pH 6.8 and 37° C., where all the molecules exist in the un-ionized form, were determined to be 1.5, 8.2, and 1.3 μg/mL, respectively; all model compounds thus have extremely poor aqueous solubility. The equilibrium solution concentrations of these compounds in the presence of selected polymers at polymer concentrations of 5 μg/mL are summarized in Table 7. At this polymer concentration, the effect of polymers on the equilibrium solubility of the model compounds is negligible.

TABLE 7
Equilibrium Solubility Values of Model Compounds in the Absence and
Presence of Polymers

| | ritonavir^{a} | efavirenz | celecoxib |
|---|---|---|---|
| drug (no polymer) | 1.3 ± 0.10 | 8.2 ± 0.17 | 1.5 ± 0.07 |
| PVP | 1.2 ± 0.05 | 8.1 ± 0.02 | 1.5 ± 0.06 |
| HPMCAS | 1.4 ± 0.09 | 8.4 ± 0.03 | 1.7 ± 0.01 |
| CMCAB | 1.3 ± 0.02 | 8.2 ± 0.06 | 1.5 ± 0.02 |
| CAP Adp 0.85 | 1.3 ± 0.01 | 8.3 ± 0.03 | 2.1 ± 0.10 |
| CAB Ad 0.81 | 1.6 ± 0.01 | 8.6 ± 0.24 | 1.6 ± 0.01 |
| Pn-IPAAmd | 1.3 ± 0.01 | 8.7 ± 0.07 | 2.3 ± 0.20 |

^{a}Values reported in ref 22.

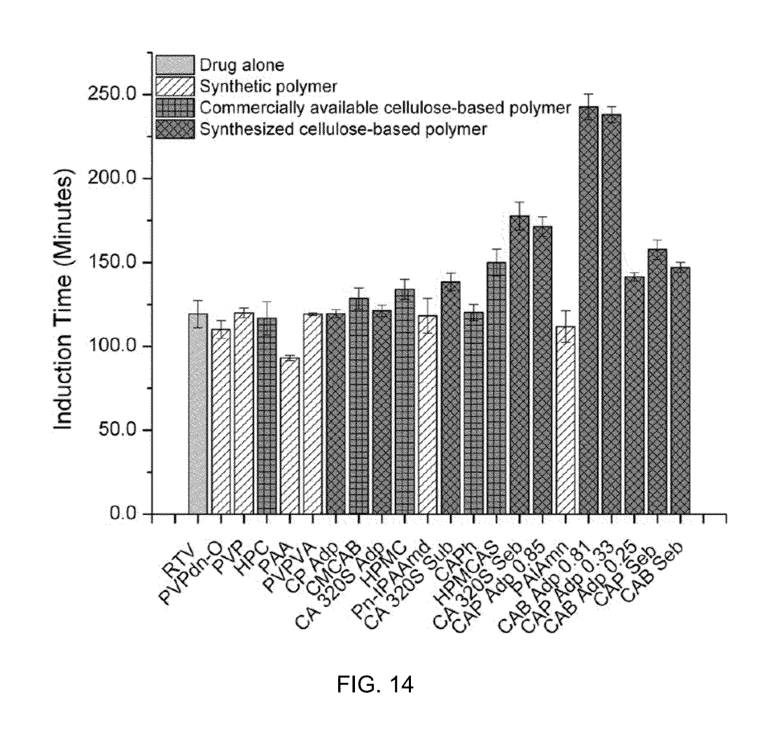

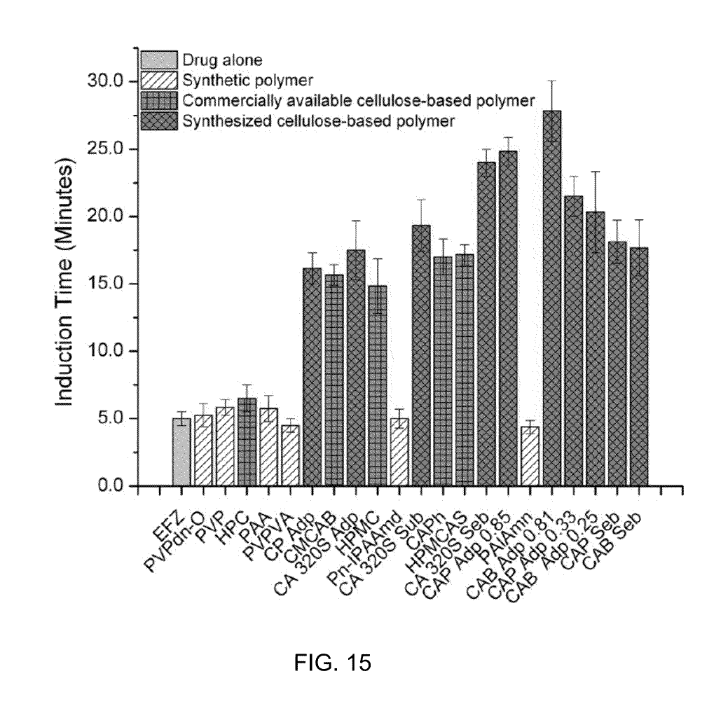

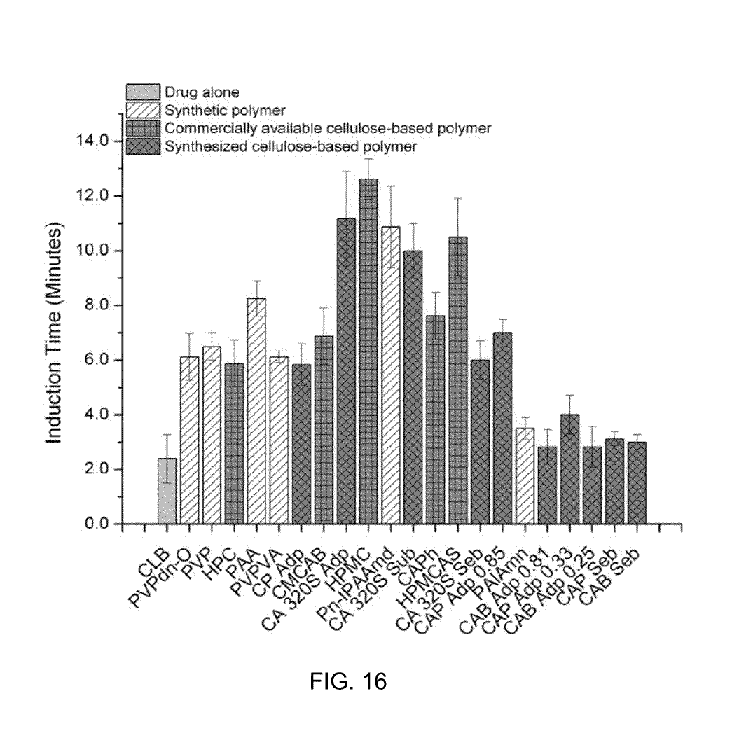

There are two crystallization pathways; crystallization of the dissolving amorphous solid, and crystallization from the supersaturated solution generated following dissolution. The crystallization tendency of the drug molecules from solution can be further inferred from their nucleation-induction times. At the beginning of the desupersaturation experiment, minimum turbidity (from light scattering measurements) was apparent, since the solution was initially clear and no particulates were present in solution. However, when nuclei began to form and grow into detectable macroscopic crystals, the turbidity of the solution began to increase. FIGS. 14-16 provide induction times for each of the compounds tested. More specifically, FIG. 14 shows the induction times for ritonavir, from unneeded desupersaturation experiments, in the absence and presence of polymers (n=3). An initial concentration corresponding to 20 μg/mL ritonavir and a polymer concentration of 5 μg/mL were used. FIG. 15 shows induction times for efavirenz from unseeded-desupersaturation experiments, in the absence and presence of polymers (n=3). An initial concentration of 19 μg/mL efavirenz and a polymer concentration of was 5 μg/mL were used. FIG. 16 shows induction times for celecoxib from unseeded-desupersaturation experiments, in the absence and presence of polymers (n=3). An initial concentration corresponding to 22 μg/mL celecoxib and a polymer concentration of 5 μg/mL were used.

Impact of Polymers on Crystal Growth Inhibition of the Model Drug Compounds. The effectiveness (E_{g}) of the polymers in inhibiting crystal growth was estimated using the following equation:

$${E}_{g} = \frac{{R}_{{g}_{0}}}{{R}_{{g}_{p}}}$$

where Rgₒ and Rgₚ are the initial bulk crystal growth rate in the absence and presence of polymer, respectively. The effectiveness crystal growth rate ratio for the polymer CA 320S Sub 0.63 (5 μg/mL) in inhibiting crystal growth of CLB at an initial concentration of 10 μg/mL was calculated to be ^{˜}6.0, which illustrates that the polymer is quite an effective inhibitor for CLB at this supersaturation. Polymers with E_{g}<1 were considered to be ineffective crystal growth inhibitors, while polymers with E_{g}>1 were deemed as showing some growth inhibition.

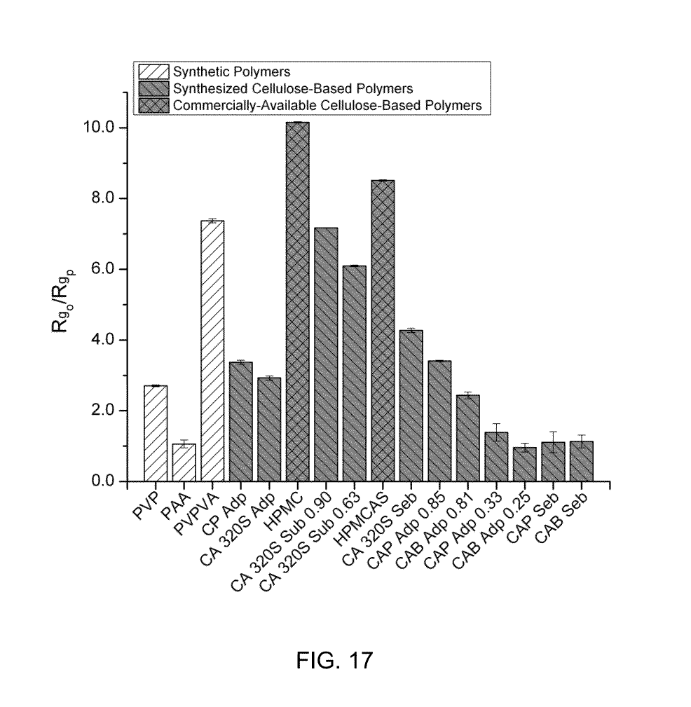

FIGS. 17-19 are graphs showing a comparison of the growth rate ratio of celecoxib, efavirenz and ritonavir, respectively, at an initial concentration of 10 μg/mL in the absence of polymer (R_{g}_{₀}) to the growth rate in the presence of all the polymers investigated (R_{g}_{ₚ}). The polymers are arranged in order of increasing hydrophobicity (left to right based on decreasing SP values). Eleven of the 13 cellulose-based polymers were recently synthesized cellulose esters (red columns), designed to span a wider range of hydrophobicities and chemical functionality than commercially available cellulose derivatives. Out of the 16 investigated polymers, 13 were effective for efavirenz, 10 were effective for ritonavir, and 12 were effective for celecoxib

$$( \frac{{R}_{{g}_{0}}}{{R}_{{g}_{p}}} > 1 ) ,$$

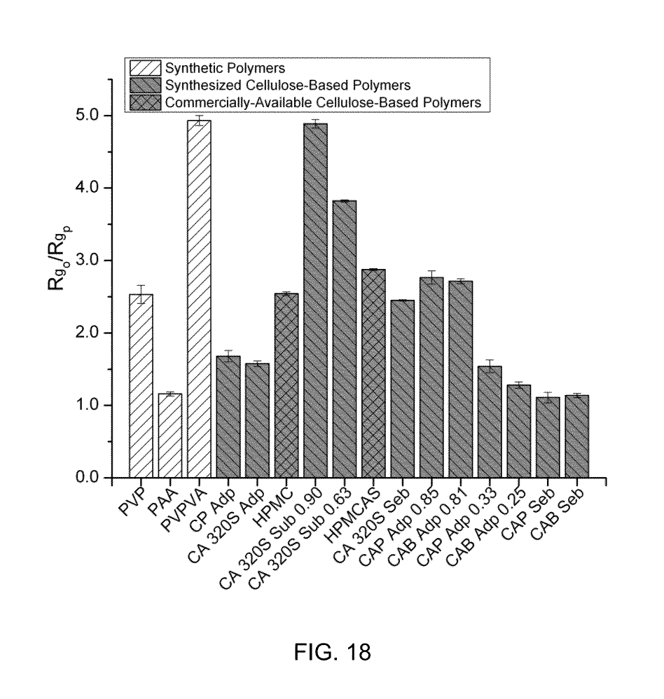

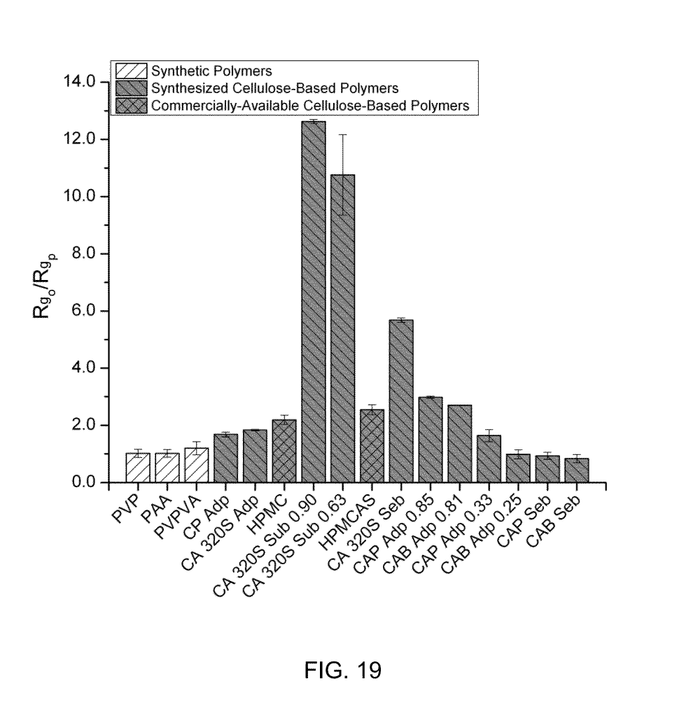

although to different extents. The cellulose-based polymers, CA 320S Sub 0.63, CA 320S Sub 0.90 and CA 320S Seb, were much more effective at inhibiting the crystal growth of ritonavir compared to commercially available polymers as well as the best of the first generation cellulose derivatives that we investigated (CAP Adp 0.85 and CAB Adp 0.81) (FIG. 19). For celecoxib, the moderately hydrophobic commercially available polymers were the most effective inhibitors (FIG. 17): HPMC>HPMCAS>PVPVA; HPMC was a very effective crystal growth inhibitor of celecoxib, inhibiting crystal growth by a factor of 10.2 while the most effective of the newly synthesized cellulose derivatives was CA 320S Sub 0.90, which inhibited the growth by a factor of about 7. A set of chemically diverse polymers was effective in inhibiting crystal growth of efavirenz (FIG. 18), whereby the two most effective polymers were PVPVA, a non-cellulose derivative, and the newly synthesized cellulose derivative, CA 320S Sub 0.90. The best performing polymers were: PVPVA≈CA 320S Sub 0.90>CAP Adp 0.85≈HPMCAS, whereby, PVPVA and CA 320S Sub 0.90, both inhibited growth by a factor of ^{˜}5.0; it should be noted that for efavirenz, the initial S was very low (1.2). In contrast, for ritonavir (FIG. 19), the moderately hydrophobic newly synthesized cellulose-based polymers were most effective: CA 320S Sub 0.90>CA 320S Sub 0.63>CA 320S Seb>CAP Adp 0.85>CAB Adp 0.81. The second-generation polymer, CA 320S Sub 0.90, was the most effective cellulose-based polymer for both efavirenz and ritonavir, inhibiting crystal growth by a factor of 4.9 and 12.8, respectively. Interestingly, the hydrophilic and synthetic polymers, PVP and PVPVA, were effective crystal growth inhibitors for celecoxib and efavirenz but not ritonavir, while PAA was ineffective for all the model compounds. Summarizing, in general the very hydrophobic newly synthesized cellulose derivatives were ineffective in inhibiting crystal growth, while the moderately hydrophobic cellulose-based polymers, both commercially available and synthesized cellulose-based polymers, were effective crystal growth inhibitors for the model drug compounds. The very hydrophilic synthetic polymers were highly variable in effectiveness.

In general, the hydrophilic polymers were less effective/ineffective in inhibiting crystal formation of the more hydrophobic compounds, efavirenz and ritonavir (SP values of 20.03 MPa^{1/2} and 21.89 MPa^{1/2}, respectively), while the more hydrophobic cellulose-based polymers (both commercially available and in-house synthesized cellulose-based polymers) were effective nucleation inhibitors. In contrast, for the more hydrophilic celecoxib (SP value of 23.38 MPa^{1/2}), the hydrophilic polymers and some of the moderately hydrophobic cellulose-based polymers were effective in increasing induction times, while the more hydrophobic polymers were ineffective. Said another way, at a similar supersaturation ratio of 1.2, ritonavir and celecoxib had slower normalized crystal growth rate (3.94 and 18.20 μg mL⁻¹ min⁻¹ m⁻², respectively), while the normalized crystal growth rate of efavirenz was significantly higher (59.32 μg mL⁻¹ min⁻¹ m⁻²), resulting in lower levels of crystal growth inhibition by the polymers for efavirenz. It is important to note that the best cellulose alkanoate-carboxyalkanoate polymers (e.g., CA 320S Sub 0.90) proved to be effective at increasing crystallization induction times, across this entire range of chemically diverse drugs.

### Example VI

### Quercetin Compositions

Quercetin (Que) and the cellulose esters CMCAB, CAAdP and HPMCAS were readily blended by spray-drying, affording amorphous solid dispersions with Que content up to 50%. Release from HPMCAS, CMCAB and CAAdP dispersions was relatively slow, probably due to the low water solubility of these polysaccharide derivatives. Cellulose derivative matrices provided pH-triggered release, in contrast with PVP matrices from which Que was released even at gastric pH. HPMCAS, CAAdP, CMCAB and PVP all inhibited Que crystallization from solution. Systems based on these cellulose ester solid dispersions are promising for development of enhanced-bioavailability, quercetin-based therapeutic and dietary supplement formulations.

A general chemical structure for quercetin is shown below:

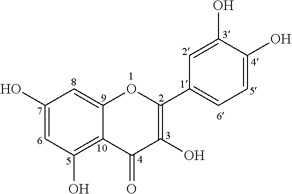 [SMILES: O=c1c(O)c(-c2ccc(O)c(O)c2)oc2cc(O)cc(O)c12]

In the measurement of drug release profiles, maximum Que concentration was fixed at 0.07 mg/mL in pH 6.8 buffer solution. The UV-vis absorption of the solution was measured and plotted vs. time. Release profiles were determined. In order to understand the influence of polymer matrix upon release, drug release profiles at pH 6.8 from Que/PVP 1/9, Que/HPMCAS 1/9, Que/CMCAB 1/9 and Que/CAAdP 1/9 solid dispersions were compared with dissolution of pure quercetin and from a physical mixture (Que/HPMCAS 1/9). Release from the Que/PVP 1/9 solid dispersion was fastest and most complete, reaching 84% within 0.5 h, while release from the Que/HPMCAS, CMCAB or CAAdP blends (1/9 SD) was slow. In contrast, dissolution of pure Que and release from Que/HPMCAS 1/9 physical mixture were much slower and less complete than that from the ASDs, reaching only 0.7% and 3% respectively after 1 h.

CAAdP suppresses quercetin crystallization in both the solid and solution phases, and provides slow release of Que at pH 6.8; it might be necessary to formulate CAAdP/Que dispersions in such a way so as to retain stabilization but enhance release rate, for example by addition of a second, miscible, more hydrophilic polymer. It is also of interest to compare Que release from these solid dispersions under conditions similar to those of the stomach, in pH 1.2 buffer. Release from the PVP amorphous blend (Que/PVP 1/9 SD blend) was substantial, reaching 80% Que release within 1 h. In contrast, only 5% Que release was observed at pH 1.2 from the quercetin amorphous blends with CAAdP. Since PVP contains a slightly basic amide group in contrast to the pendent carboxyls of the cellulose esters, this result is exactly what we would expect chemically. Thus, it could be expected that CAAdP would isolate Que from the stomach contents much more effectively than would PVP.

### Example VII

### Ritonavir Nanoparticle or Nanodroplet Compositions

Stable amorphous and preferably uniform nanoparticles of poorly soluble drugs can be formed and kept in solution for a biologically relevant time scale to increase the solubility of poorly soluble lipophilic drug molecules. For example, drug-rich amorphous nanoparticles can be formed by dissolving an initially solid amorphous dispersion of a lipophilic compound molecularly mixed with a suitable polymer at an appropriate drug:polymer ratio into an aqueous solution. Any of the drugs and polymers described in this specification can be prepared to provide such compositions. The resultant amorphous nanodroplets which are dispersed in the aqueous medium vary on size and stability, dependent on the type of polymer used in the formulation. By appropriate selection of the polymer, these amorphous nanodroplets or nanoparticles of a size of less than 200 nanometers can be produced and stabilized over biologically relevant time scales. Nanodroplets or nanoparticles having a size ranging from about 1-200 nm are preferred, such as from about 10-175 nm, or from about 20-150 nm, such as from 25-125 nm, or from about 30-110 nm, or from about 50-100 nm, such as from about 75-90 nm and so on. Indeed, the drugs in composition embodiments of the invention can have a particle size ranging from 1-1,000 nm, such as from 100 to 750 nm, or from 250 to 500 nm, for example. These nanodroplets and nanoparticles are expected to enhance the delivery of poorly water soluble drugs by virtue of their small size and ability to rapidly equilibrate within the bulk phase, as well as being directly absorbed in the body.

Polymers with appropriate properties to produce smaller nanodroplets include in particular those polymers which can become charged. Preferred polymers include cellulose derivatives such as hydroxypropyl methyl cellulose acetate succinate, cellulose acetate propionate adipate, and cellulose acetate phthalate; methacrylates such as Eudgragit E100 and Eudragit L100; polyacrylic acid, and any other synthetic or naturally-derived water soluble polymer capable of forming an amorphous dispersion with the drug and becoming charged at a physiologically relevant pH. Surfactants and polymeric surfactants including anionic surfactants (for example, sodium lauryl sulfate), cationic surfactants (for example benzalkonium chloride), and non-ionic surfactants (for example, polyethylene glycol based surfactants such as the Pluronics), can be used to stabilize the nanodroplets against size enlargement and promote the formation of smaller droplets. Nanodroplet formation may derive from many classes of therapeutic compounds, including but not limited to antifungals, cancer therapeutics, antivirals, antibiotics, antihistamines, lipid lowering drugs, immunosuppressants, cardiovascular drugs, and drugs acting on the central nervous system including for Alzheimer's and Parkinson's diseases. Even further, any combination of drug(s) and polymer(s) can be formulated to provide a nanoparticle/nanodroplet type composition in accordance with the invention.

Dynamic light scattering (DLS) was used to monitor changes in particle size as a function of time during de-supersaturation experiments. DLS experiments were performed using a Nano-Zetasizer (Nano-ZS) and its software, dispersion technology software (DTS). A 173° backscatter detector was used in order to minimize the signal from large dust particles or other large contaminants. A quartz flow-through cuvette, coupled with a pump was used for continuous sampling. Supersaturated solutions were generated by adding a small volume of pre-dissolved ritonavir in methanol to 100 mM sodium phosphate buffer at pH 6.8.

Particle size experiments were performed in the absence and presence of pre-dissolved polymers. As an example, ritonavir was used at an initial concentration of 25 μg/mL with a polymer concentration of 50 μg/mL. The buffer solution was allowed to equilibrate at 37° C. prior to addition of solubilized ritonavir. Data collection began after generation of supersaturation. A stir-plate was used to stir the solution at a speed of 400 rpm.

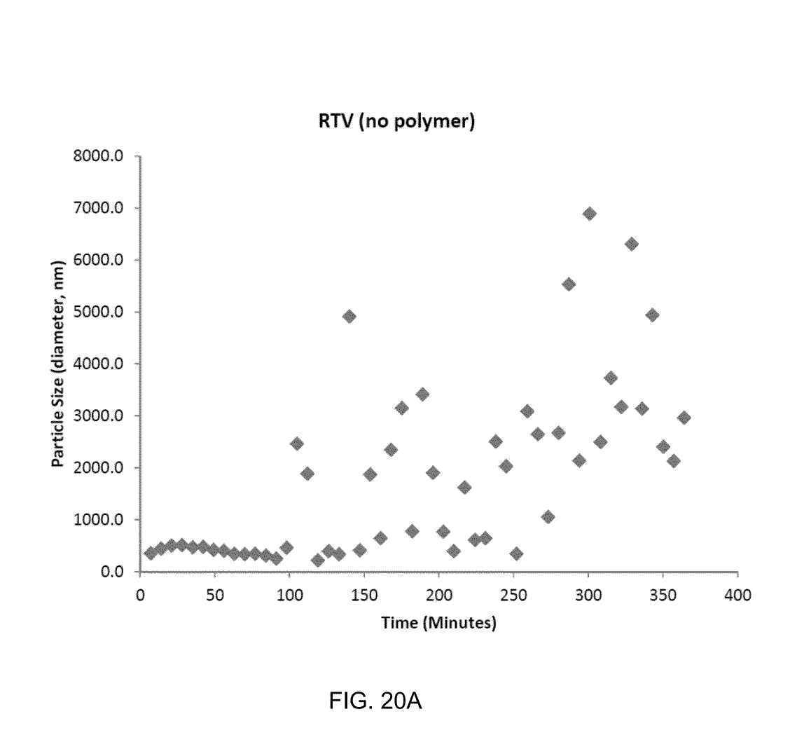

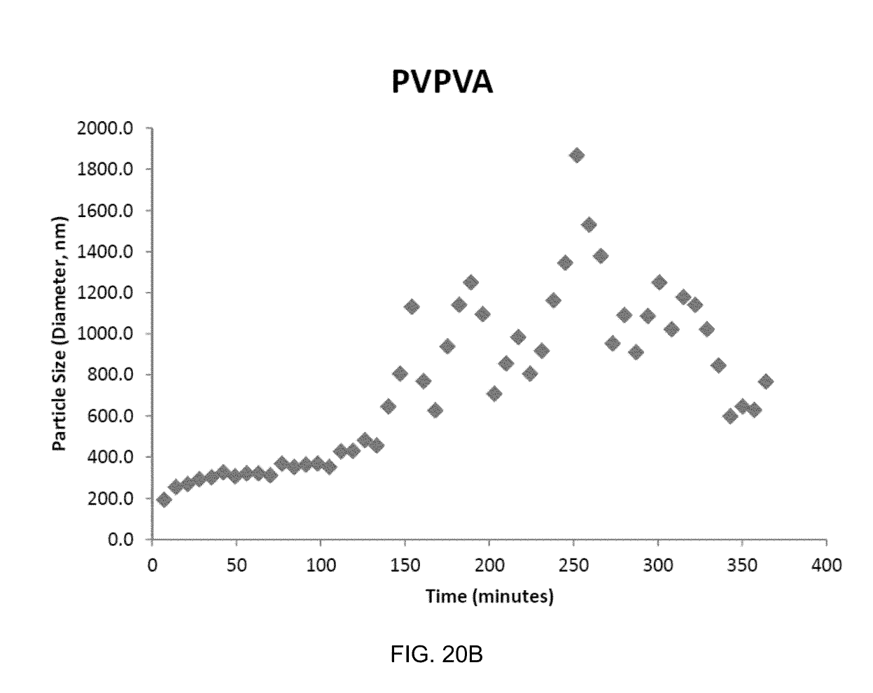

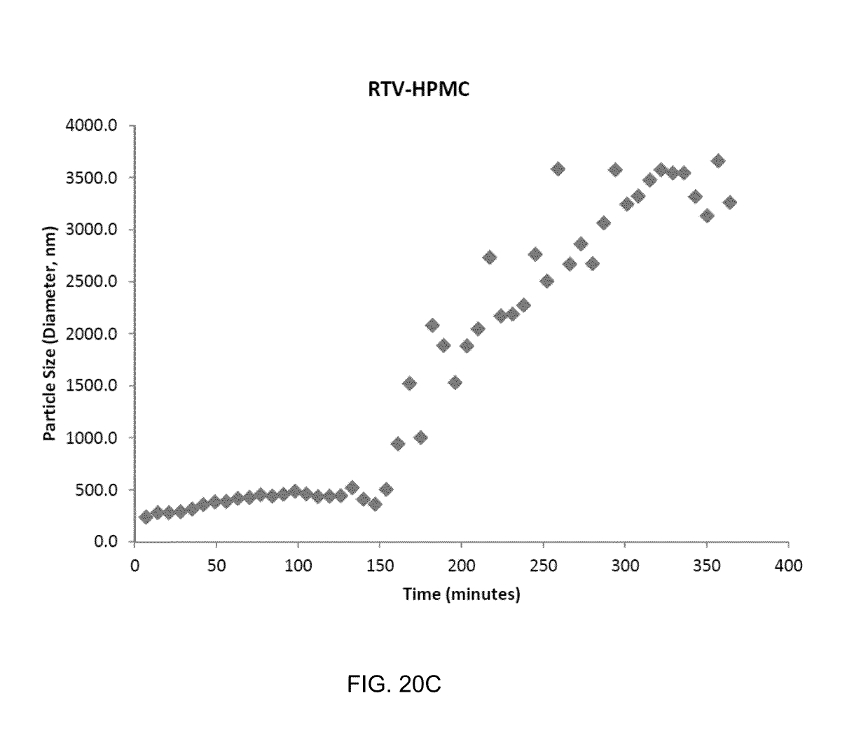

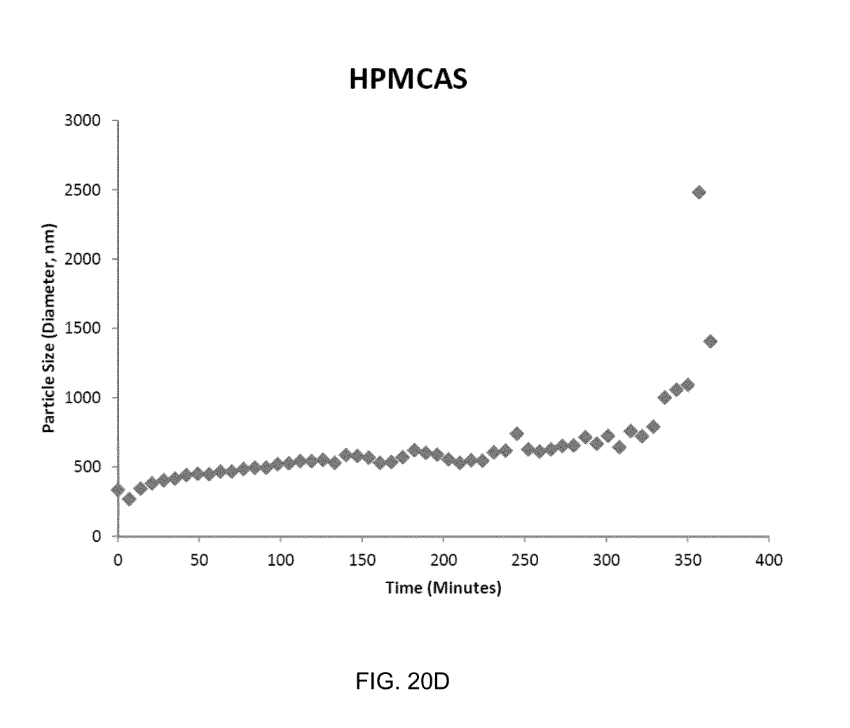

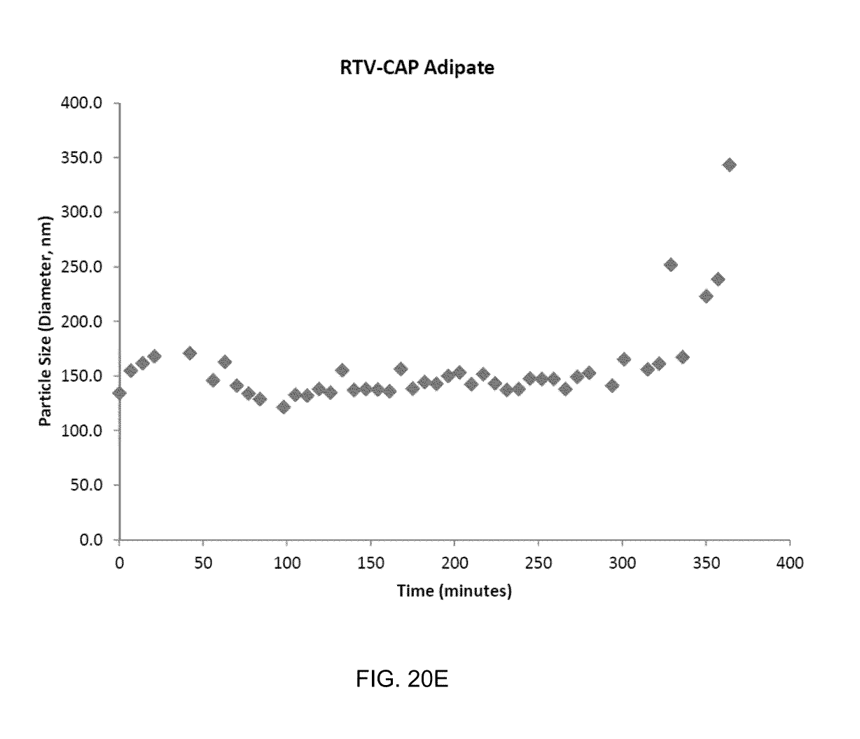

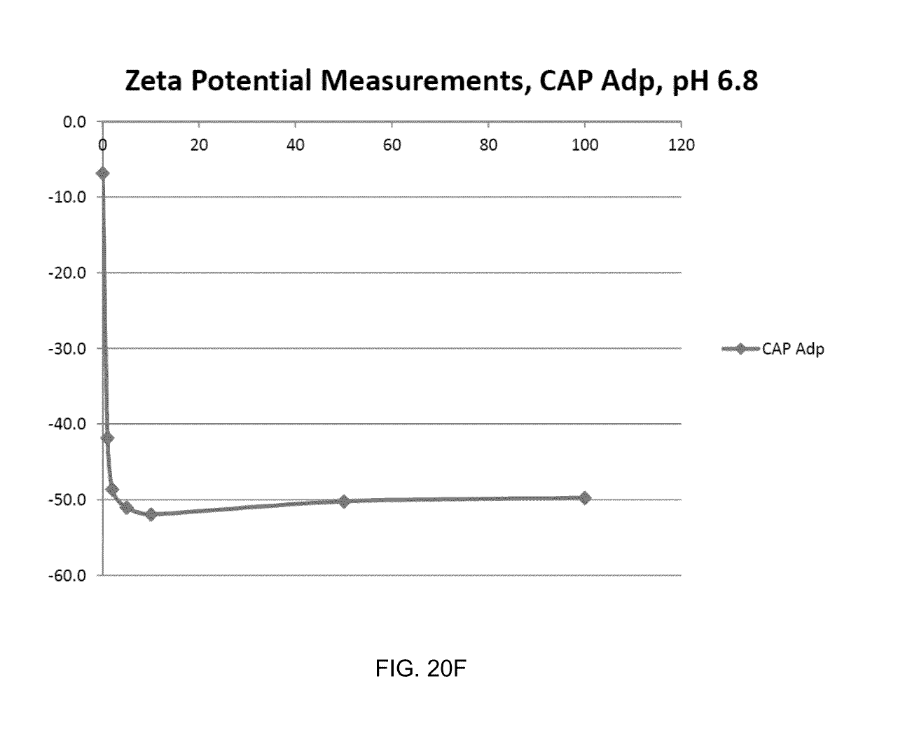

In one embodiment, DLS indicated that particulates were formed from the supersaturation solutions of ritonavir. As shown in FIG. 20A, in the absence of polymer, the size of ritonavir particles was maintained at ^{˜}350-450 nm for ^{˜}100 minutes, thereafter, the particles in solution grew and their sizes varied significantly (500->6000 nm). The wide range of particle sizes observed may have been due to particle aggregation. As shown in FIGS. 20B-C, in the presence of the non-ionized polymers (at pH 6.8), poly(vinylpyrrolidone vinyl acetate) (PVPVA) and hydroxypropyl methyl cellulose (HPMC), particles with sizes ranging from ^{˜}300-450 nm were also formed. The polymers were unable to prevent the growth and aggregation of the particulates in solution. In contrast, as shown in FIGS. 20D-E, hydroxypropyl methyl cellulose acetate succinate (HPMCAS) and cellulose acetate propionate 504-0.2 adipate (CAP Adp) were effective in preventing the growth and aggregation of the particles in solution, although to varying extents. Both HPMCAS and CAP Adp have ionizable carboxylic acid functional groups and are partially ionized at pH 6.8. In the presence of CAP Adp, smaller particle sizes ^{˜}150 nm were generated from the supersaturation solution of ritonavir. As shown in FIG. 20F, Zeta potential measurements were performed to quantify the surface charge on ritonavir crystals in dissolved CAP Adp (50 μg/mL) at pH 6.8. The zeta potential is a function of the surface coverage by charged species at a given pH, which theoretically is determined by the activity of the species in solution. The zeta potential of the seed crystal-polymer suspension at pH 6.8 was −50.2 mV, due to the ionization of the carboxylic acid function of the adipate substitution group. Adsorbed CAP Adp on ritonavir particles prevents aggregation of particulates due to factors such as repulsion between ionized groups and improved interaction with the solvent.

Methods of using the polymers and compositions of the invention are also included within the scope of the invention. For example, a method of treating a subject having a disease chosen from at least one of AIDS, HIV, or cancer is included, said method comprising administering an effective amount of a composition to the subject for a time and under conditions sufficient to treat the disease, said composition comprising: at least one amorphous drug with a solubility of less than about 1 mg/mL; at least one first polymer chosen from cellulose esters of formula I:

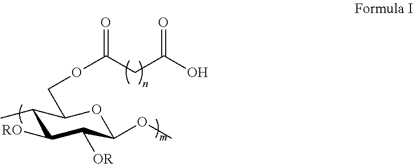

wherein n of the ω-carboxyalkanoyl group,

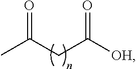

is 1, 2, 3, 4, 5, 6, 7, 8, 9, or 10; wherein R is chosen from: a hydrogen atom; and an alkanoyl group; and

wherein the polymer comprises m repeating units ranging from 1 to 1,000,000. Preferred polymers can have n repeating units for example ranging from 10 to 100,000, or from 100 to 1,000.

Methods of embodiments of the invention can comprise administering a composition with a drug and polymer wherein there is a degree of substitution with respect to the ω-carboxyalkanoyl group

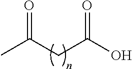

of the polymer of 0.05 up to 2, such as near 1. Even further, the methods can comprise administering a composition, wherein there is a total degree of substitution of the alkanoyl group and the ω-carboxyalkanoyl group of the polymer of at least 2.0.

In preferred methods, the alkanoyl group of the polymer can be chosen from at least one of acetyl, propionyl, butyryl, valeroyl, hexanoyl, nonanoyl, decanoyl, lauroyl, palmitoyl, and stearoyl groups. Likewise, in preferred methods, the ω-carboxyalkanoyl group of the polymer can be chosen from at least one of succinoyl, glutaroyl, adipoyl, sebacyl, and suberyl groups.

Methods of the invention include administering one or more drugs in combination with any of the polymers identified in this specification. The amount and dosage schedule of the drug is not critical and can be performed according to any conventional means or method available for a particular drug. Dosage amounts can range for example up to 1000 mg, such as 10 mg, 20 mg, 30 mg, 40 mg, 50 mg, 60 mg, 70, mg, 80 mg, 90 mg, 100 mg, 150 mg, 200 mg, 250 mg, 300 mg, 350 mg, 400 mg, 450 mg, 500 mg and so one. The drugs in any one or more of these dosage amounts can be administered 1×, 2×, 3×, 4×, 5×, and so on each day, hour, week, month, or year depending on the drug. The compositions of the invention can be administered in any manner, such as by oral, parenteral, intramuscular, intravenous, cutaneous, subcutaneous, nasal, intraocular, transepithelial, intraperitoneal, topical (such as dermal, ocular, rectal, nasal, inhalation and aerosol), rectal, and/or stomach tube routes. Pharmaceutical compositions can be prepared in any acceptable form, such as in the form of capsules, powder, tablets, a suspension, or solution, optionally in admixture with a pharmaceutically acceptable carrier or diluents. Forms and dosages of appropriate pharmaceutical compositions that are appropriate for administration to humans and other warm blooded mammals can be formulated based on the information provided in this specification in combination with techniques well known in the art.

The present invention has been described with reference to particular embodiments having various features. It will be apparent to those skilled in the art that various modifications and variations can be made in the practice of the present invention without departing from the scope or spirit of the invention. One skilled in the art will recognize that the features of embodiments of the invention may be used singularly or in any combination based on the requirements and specifications of a given application or design, and one or more elements, constituents, or process steps may be omitted, incorporated, or altered as desired. Other embodiments of the invention will be apparent to those skilled in the art from consideration of the specification and practice of the invention. It is intended that the specification and examples be considered as exemplary and that variations that do not depart from the essence of the invention are intended to be within the scope of the invention.

Further, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. It should be evident that the particular illustrative embodiments disclosed above may be altered or modified and all such variations are considered within the scope and spirit of the present invention. While systems and methods are described in terms of “comprising,” “having,” “containing,” or “including” various components or steps, the systems and methods can also “consist essentially of” or “consist of” one or more of the various components or steps. All numbers and ranges disclosed in this specification may vary by some amount. Whenever a numerical range with a lower limit and an upper limit is disclosed, any number and any included range falling within the range is specifically disclosed. In particular, every range of values disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one, at least one, or more than one of the element it introduces. All references cited in this specification are hereby incorporated by reference herein in their entireties. If there is any conflict in the usages of a word or term in this specification and one or more patent or other documents cited herein, the definitions consistent with this specification should be adopted.

1. A composition comprising:
at least one amorphous drug with a solubility of less than about 1 mg/mL;
at least one first polymer chosen from cellulose esters of formula I: 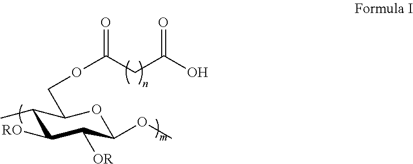
wherein n is 1, 2, 3, 4, 5, 6, 7, 8, 9, or 10;
wherein R is chosen from:
a hydrogen atom; and
an alkanoyl group;
wherein m ranges from 1 to 6,000.

2. The composition of claim 1, wherein there is a degree of substitution with respect to the ω-carboxyalkanoyl group 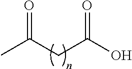
of 0.05-2.

3. The composition of claim 2, wherein there is a degree of substitution with respect to the ω-carboxyalkanoyl group 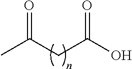
of 1 or near 1.

4. The composition of claim 2, wherein there is a degree of substitution with respect to the ω-carboxyalkanoyl group 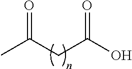
of 0.5-1.

5. The composition of claim 1, wherein there is a total degree of substitution of the alkanoyl group and the ω-carboxyalkanoyl group of at least 2.0.

6. The composition of claim 1, wherein the alkanoyl group is chosen from at least one of acetyl, propionyl, butyryl, valeroyl, hexanoyl, nonanoyl, decanoyl, lauroyl, palmitoyl, and stearoyl groups.

7. The composition of claim 1, wherein the ω-carboxyalkanoyl group is chosen from at least one of succinoyl, glutaroyl, adipoyl, sebacyl, and suberyl groups.

8. The composition of any of claims 1-7, wherein bioavailability of the drug is enhanced above that of the drug by itself.

9. The composition of claim 1, wherein drug solution concentration achieved from the composition is higher than the solubility of the drug in buffer solutions that simulate small intestine contents.

10. The composition of claim 7, wherein drug solution concentration from the composition is higher than the solubility of the drug in pH 6.8 buffer solutions.

11. The composition of claim 1 further comprising a second polymer.

12. The composition of claim 11, wherein the second polymer is selected such that it modifies release rate of the drug.

13. The composition of claim 11, wherein the second polymer is more water soluble than the first polymer.

14. The composition of claim 11, wherein solution concentration of the drug is enhanced as compared with that of either the first or second polymer by itself.

15. The composition of claim 11, wherein the second polymer is chosen from at least one of poly(vinylpyrrolidinone), hydroxypropyl methylcellulose, poly(ethylene glycol), and poly(propylene glycol).

16. The composition of claim 1, wherein the drug is amorphous for at least 1 year.

17. The composition of claim 1, wherein the drug is amorphous for at least 4 years.

18. The composition of claim 1, wherein upon dispersion in buffer solutions that simulate the small intestine, the drug dissolves to a maximum concentration, and at least 90% of that concentration is maintained for at least 24 h.

19. The composition of claim 10, wherein the buffer solution is a phosphate buffer with pH 6.8.

20. The composition of claim 1, wherein the drug is chosen from at least one of ritonavir, efavirenz, etravirine, celecoxib, and clarithromycin.

21. The composition of claim 1, wherein the drug is chosen from at least one of curcumin, ellagic acid, quercetin, naringenin, and resveratrol.

22. The composition of claim 1, wherein the drug is chosen from antihypertensives, antianxiety agents, anticlotting agents, anticonvulsants, blood glucose lowering agents, decongestants, antihistamines, antitussives, antineoplastics, beta blockers, anti-inflammatories, antipsychotic agents, cognitive enhancers, cholesterol reducing agents, triglyceride reducing agents, anti-atherosclerotic agents, anti-obesity agents, autoimmune disorder agents, anti-impotence agents, anti-bacterial and anti-fungal agents, hypnotic agents, anti-Parkinsonism agents, anti-Alzheimer's disease agents, antibiotics, antidepressants, antiviral agents, glycogen phosphorylase inhibitors, protease inhibitors, and cholesteryl ester transfer protein inhibitors.

23. A method of treating a subject having a disease chosen from at least one of AIDS, HIV, or cancer, said method comprising administering an effective amount of a composition to the subject for a time and under conditions sufficient to treat the disease, said composition comprising:
at least one amorphous drug with a solubility of less than about 1 mg/mL;
at least one first polymer chosen from cellulose esters of formula I: 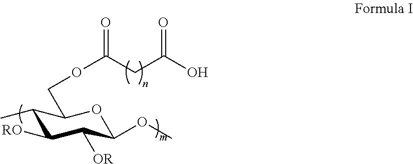
wherein n is 1, 2, 3, 4, 5, 6, 7, 8, 9, or 10;
wherein R is chosen from:
a hydrogen atom; and
an alkanoyl group; and
wherein m ranges from 1 to 6,000.

24. The method of claim 23, wherein there is a degree of substitution with respect to the ω-carboxyalkanoyl group 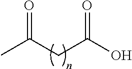
of 0.05-2.

25. The method of claim 23, wherein there is a degree of substitution with respect to the ω-carboxyalkanoyl group 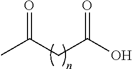
of 1 or near 1.

26. The method of claim 23, wherein there is a degree of substitution with respect to the ω-carboxyalkanoyl group 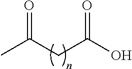
of 0.5-1.

27. The method of claim 23, wherein there is a total degree of substitution of the alkanoyl group and the ω-carboxyalkanoyl group of at least 2.0.

28. The method of claim 23, wherein the alkanoyl group is chosen from at least one of acetyl, propionyl, butyryl, valeroyl, hexanoyl, nonanoyl, decanoyl, lauroyl, palmitoyl, and stearoyl groups.

29. The method of claim 23, wherein the ω-carboxyalkanoyl group is chosen from at least one of succinoyl, glutaroyl, adipoyl, sebacyl, and suberyl groups.

30. A composition for use in the treatment of a disease chosen from at least one of AIDS, HIV, or cancer by administering an effective amount of the composition to a subject with the disease, wherein the composition comprises:
at least one amorphous drug with a solubility of less than about 1 mg/mL;
at least one first polymer chosen from cellulose esters of formula I: 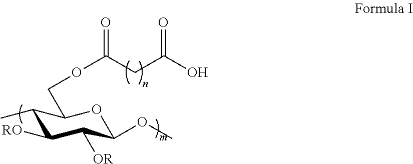
wherein n is 1, 2, 3, 4, 5, 6, 7, 8, 9, or 10;
wherein R is chosen from:
a hydrogen atom; and
an alkanoyl group; and
wherein m ranges from 1 to 6,000.

31. The composition of claim 30, wherein there is a total degree of substitution of the alkanoyl group and the ω-carboxyalkanoyl group of at least 2.0.

32. The composition of claim 30, wherein the alkanoyl group is chosen from at least one of acetyl, propionyl, butyryl, valeroyl, hexanoyl, nonanoyl, decanoyl, lauroyl, palmitoyl, and stearoyl groups.

33. The composition of claim 30, wherein the ω-carboxyalkanoyl group is chosen from at least one of succinoyl, glutaroyl, adipoyl, sebacyl, and suberyl groups.

34. The composition of any of claim 1-22 or 30-33, wherein the amorphous drug is in the form of nanoparticles of 200 nm or less.

35. The composition of any of claim 1-22 or 30-33, wherein the amorphous drug is in the form of nanodroplets of 200 nm or less.

36. The method of any of claims 23-29, wherein the amorphous drug is in the form of nanoparticles of 200 nm or less.

37. The method of any of claims 23-29, wherein the amorphous drug is in the form of nanodroplets of 200 nm or less.

38. A cellulose ester of formula I: 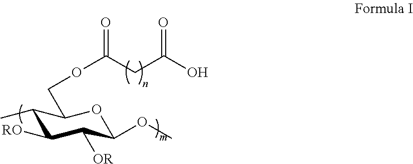
wherein n is 2, 3, 4, 6, or 8 to provide a ω-carboxyalkanoyl group chosen from succinoyl, glutaroyl, adipoyl, sebacyl, and suberyl groups;
wherein R is chosen from:
a hydrogen atom; and
an alkanoyl group chosen from acetyl, propionyl, butyryl, valeroyl, and hexanoyl groups; and
wherein there is a total degree of substitution of the alkanoyl group and the ω-carboxyalkanoyl group of at least 2.0; and
wherein m ranges from 1 to 6,000.
